(19) United States
(12) Patent Application Publication
(10) Pub. No.: US 2006/0046968 A1
(43) Pub. Date: Mar. 2, 2006

(54) Prodrug substituted benzoxazoles as estrogenic agents

(75) Inventors: Sayed Elmarakby, Newtown, PA (US); Ping Cai, New City, NY (US); Appavu Chandrasekaran, Plainsboro, NJ (US); Mark Ruppen, Garnerville, NY (US); Rasmy Talaat, West Chester, PA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/210,427

(22) Filed: Aug. 24, 2005

Related U.S. Application Data
(60) Provisional application No. 60/604,835, filed on Aug. 26, 2004.

(51) Int. Cl. A61K 31/7052; A61K 31/423; C07D 263/52

(52) U.S. Cl. 514/23; 514/375; 536/17.4; 548/216

(57) ABSTRACT

This invention provides estrogen receptor modulators of formula I, having the structure 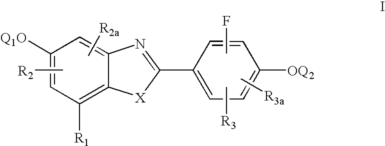 wherein
Q, Q₂, R₁, R₂, R₂ₐ, R₃, R₃ₐ, and X as defined in the specification, or a pharmaceutically acceptable salt thereof.

### CROSS REFERENCE TO RELATED APPLICATION

This This application claims priority benefit of U.S. Provisional Application Ser. No. 60/604,835, filed Aug. 26, 2004, which is hereby incorporated by reference in its entirety.

### BACKGROUND OF THE INVENTION

This invention relates to prodrug derivatives of substituted benzoxazoles, which are useful as estrogenic agents.

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation 351-361 (2000)]. A class of “coregulatory” proteins also can interact with the ligand-bound receptor and further modulate its transcriptional activity [McKenna, et al., Endocrine Reviews 20: 321-344 (1999)]. It has also been shown that estrogen receptors can suppress NF_{K}B-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Peizer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERα has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality, there is a continuum between estrogen receptor agonist and estrogen receptor antagonist activity and indeed, some compounds behave as estrogen receptor agonists in some tissues and estrogen receptor antagonists in others. These compounds with mixed activity are called selective estrogen receptor modulators (SERMS) and are therapeutically useful agents (e.g. EVISTA®) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist, which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists, 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

As mentioned above, estrogens affect a panoply of biological processes. In addition, where gender differences have been described (e.g., disease frequencies, responses to challenge, etc.), it is possible that the explanation involves the difference in estrogen levels between males and females.

Compounds having estrogenic activity are disclosed in U.S. patent application Ser. No. 10/309,699 filed Dec. 4, 2002, now U.S. Pat. No. 6,794,403, and in WO 03/050095, which are incorporated herein by reference in their entireties.

### DESCRIPTION OF THE INVENTION

This invention provides estrogenic compounds of formula I having the structure: 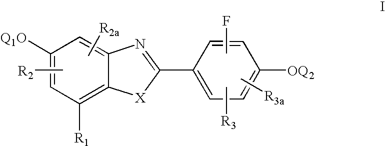
wherein:
Q₁ and Q₂ are independently H, a modified or unmodified hexose residue, or S(O)ₜ—OH, provided that Q₁ and Q₂ are not both H;
   t is 0, 1 or 2;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
   R₇ is hydrogen, alkyl of 1-6 carbon atoms, aryl of 6-10 carbon atoms, —COR₅, —CO₂R₅ or —SO₂R₅;
or a pharmaceutically acceptable salt thereof, which are useful as estrogenic agents.

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic moiety. Salts may also be formed from organic and inorganic bases, such as alkali metal salts (for example, sodium, lithium, or potassium), alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when a compound of this invention contains an acidic moiety.

The terms “alkyl”, “alkenyl”, and “alkynyl” include both branched and straight chain moieties. Examples include methyl, ethyl, propyl, butyl, isopropyl, sec-butyl, tert-butyl, vinyl, allyl, acetylene, 1-methyl vinyl, and the like. When alkyl or alkenyl moieties are substituted, they may typically be mono-, di-, tri- or persubstituted. Examples for a halogen substituent include 1-bromo vinyl, 1-fluoro vinyl, 1,2-difluoro vinyl, 2,2-difluorovinyl, 1,2,2-trifluorovinyl, 1,2-dibromo ethane, 1,2 difluoro ethane, 1-fluoro-2-bromo ethane, CF₂CF₃, CF₂CF₂CF₃, and the like. The term “halogen” includes bromine, chlorine, fluorine, and iodine. The term “aryl” includes an aromatic of 6-10 carbon atoms e.g., phenyl, 1-naphthyl, or 2-naphthyl. Preferred 5-6 membered heterocyclic rings include furan, thiophene, pyrrole, isopyrrole, pyrazole, imidazole, triazole, dithiole, oxathiole, isoxazole, oxazole, thiazole, isothiazolem oxadiazole, furazan, oxatriazole, dioxazole, oxathiazole, tetrazole, pyran, pyridine, pyridazine, pyrimidine, pyrazine, triazine, oxazine, oxathiazine, or oxadiazine. More preferred heterocyclic rings are furan, thiophene, or thiazole.

Of the compounds of this invention, it is preferred that the compound of formula I has the structure: 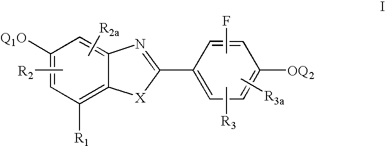
wherein:
Q₁ and Q₂ are independently H, a modified or unmodified hexose residue, or S(O)ₜ—OH, provided that Q₁ and Q₂ are not both H;
   t is 2;
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR6;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
   X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof.

It is more preferred that X is O, and still more preferred that X is O, and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

It is more preferred that Q₁ and Q₂ are selected from —SO₃H and glucuronide residues.

In some particularly preferred embodiments, the compound is a mono- or di-sulfate derivative, a mono- or di-glucuronide derivative, or a glucuronide-sulfate derivative of 2-(3′-fluoro-4′-hydroxyphenyl)-7-vinyl-1,3-benzoxazol-5-ol, or a pharmaceutically acceptable salt thereof. In some embodiments, the compound is 2-(3′-fluoro-4′-glucuronide phenyl)-7-vinyl-1,3-benzoxazol-5-ol; 2-(3′-fluoro-4′-sulfate phenyl)-7-vinyl-1,3-benzoxazol-5-ol; 2-(3′-fluoro-4′-hydroxy phenyl)-7-vinyl-1,3-benzoxazol-5-glucuronide; 2-(3′-fluoro-4′-hydroxy phenyl)-7-vinyl-1,3-benzoxazol-5-sulfate; 2-(3′-fluoro-4′-glucuroride phenyl)-7-vinyl-1,3-benzoxazol-5-glucuronide; 2-(3′-fluoro-4′-glucuronide phenyl)-7-vinyl-1,3-benzoxazol-5-sulfate; 2-(3′-fluoro-4′-sulfate phenyl)-7-vinyl-1,3-benzoxazol-5-glucuronide; 2-(3′-fluoro-4′-sulfate phenyl)-7-vinyl-1,3-benzoxazol-5-sulfate; 2-(2′-fluoro-4′-glucuronide phenyl)-7-vinyl-1,3-benzoxazol-5-ol; 2-(2′-fluoro-4′-sulfate phenyl)-7-vinyl-1,3-benzoxazol-5-ol; 2-(2′-fluoro-4′-hydroxy phenyl)-7-vinyl-1,3-benzoxazol-5-glucuronide; 2-(2′-fluoro-4′-hydroxy phenyl)-7-vinyl-1,3-benzoxazol-5-sulfate; 2-(2′-fluoro-4′-glucuronide phenyl)-7-vinyl-1,3-benzoxazol-5-glucuronide; 2-(2′-fluoro-4′-glucuronide phenyl)-7-vinyl-1,3-benzoxazol-5-sulfate; 2-(2′-fluoro-4′-sulfate phenyl)-7-vinyl-1,3-benzoxazol-5-glucuronide; 2-(2′-fluoro-4′-sulfate phenyl)-7-vinyl-1,3-benzoxazol-5-sulfate; 2-(2′,3′-difluoro-4′-glucuronide phenyl)-7-vinyl-1,3-benzoxazol-5-ol; 2-(2′,3′-difluoro-4′-sulfate phenyl)-7-vinyl-1,3-benzoxazol-5-ol; 2-(2′,3′-difluoro-4′-hydroxyphenyl)-7-vinyl-1,3-benzoxazol-5-glucuronide; 2-(2′,3′-difluoro-4′-hydroxyphenyl)-7-vinyl-1,3-benzoxazol-5-sulfate; 2-(2′,3′-difluoro-4′-glucuronide phenyl)-7-vinyl-1,3-benzoxazol-5-glucuronide; 2-(2′,3′-difluoro-4′-glucuronide phenyl)-7-vinyl-1,3-benzoxazol-5-sulfate 2-(2′,3′-difluoro-4′-sulfate phenyl)-7-vinyl-1,3-benzoxazol-5-glucuronide; 2-(2′,3′-difluoro-4′-sulfate phenyl)-7-vinyl-1,3-benzoxazol-5-sulfate; 4-bromo-2-(3′-fluoro-4′-glucuronide phenyl)-7-vinyl-1,3-benzoxazol-5-ol; 4-bromo-2-(3′-fluoro-4′-sulfate phenyl)-7-vinyl-1,3-benzoxazol-5-ol; 4-bromo-2-(3′-fluoro-4′-hydroxyphenyl)-7-vinyl-1,3-benzoxazol-5-glucoronide; 4-bromo-2-(3′-fluoro-4′-hydroxyphenyl)-7-vinyl-1,3-benzoxazol-5-sulfate; 4-bromo-2-(3′-fluoro-4′-glucuronide phenyl)-7-vinyl-1,3-benzoxazol-5-glucuronide; 4-bromo-2-(3′-fluoro-4′-glucuronide phenyl)-7-vinyl-1,3-benzoxazol-5-sulfate; 4-bromo-2-(3′-fluoro-4′-sulfate phenyl)-7-vinyl-1,3-benzoxazol-5-glucuronide; 4-bromo-2-(3′-fluoro-4′-sulfate phenyl)-7-vinyl-1,3-benzoxazol-5-sulfate; 4,6-dibromo-2-(3′-fluoro-4′-glucuronide phenyl)-7-vinyl-1,3-benzoxazol-5-ol; 4,6-dibromo-2-(3′-fluoro-4′-sulfate phenyl)-7-vinyl-1,3-benzoxazol-5-ol; 4,6-dibromo-2-(3′-fluoro-4′-hydroxyphenyl)-7-vinyl-1,3-benzoxazol-5-glucuronide; 4,6-dibromo-2-(3′-fluoro-4′-hydroxyphenyl)-7-vinyl-1,3-benzoxazol-5-sulfate; 4,6-dibromo-2-(3′-fluoro-4′-glucuronide phenyl)-7-vinyl-1,3-benzoxazol-5-glucuronide; 4,6-dibromo-2-(3′-fluoro-4′-glucuronide phenyl)-7-vinyl-1,3-benzoxazol-5-sulfate; 4,6-dibromo-2-(3′-fluoro-4′-sulfate phenyl)-7-vinyl-1,3-benzoxazol-5-glucuronide; 4,6-dibromo-2-(3′-fluoro-4′-sulfate phenyl)-7-vinyl-1,3-benzoxazol-5-sulfate; 7-(1-bromovinyl)-2-(2′-fluoro-4′-glucuronide phenyl)-1,3-benzoxazol-5-ol; 7-(1-bromovinyl)-2-(2′-fluoro-4′-sulfate phenyl)-1,3-benzoxazol-5-ol; 7-(1-bromovinyl)-2-(2′-fluoro-4′-hydroxyphenyl)-1,3-benzoxazol-5-glucuronide; 7-(1-bromovinyl)-2-(2′-fluoro-4′-hydroxyphenyl)-1,3-benzoxazol-5-sulfate; 7-(1-bromovinyl)-2-(2′-fluoro-4′-glucuronide phenyl)-1,3-benzoxazol-5-glucuronide; 7-(1-bromovinyl)-2-(2′-fluoro-4′-glucuronide phenyl)-1,3-benzoxazol-5-sulfate; 7-(1-bromovinyl)-2-(2′-fluoro-4′-sulfate phenyl)-1,3-benzoxazol-5-glucuronide; 7-(1-bromovinyl)-2-(2′-fluoro-4′-sulfate phenyl)-1,3-benzoxazol-5-sulfate; 7-(1-bromovinyl)-2-(2′,3′-difluoro-4′-glucuronide phenyl)-1,3-benzoxazol-5-ol; 7-(1-bromovinyl)-2-(2+,3′-difluoro-4′-sulfate phenyl)-1,3-benzoxazol-5-ol; 7-(1-bromovinyl)-2-(2′,3′-difluoro-4′-hydroxyphenyl)-1,3-benzoxazol-5-glucuronide; 7-(1-bromovinyl)-2-(2′,3′-difluoro-4′-hydroxyphenyl)-1,3-benzoxazol-5-sulfate; 7-(1-bromovinyl)-2-(2′,3′-difluoro-4′-glucuronide phenyl)-1,3-benzoxazol-5-glucuronide; 7-(1-bromovinyl)-2-(2′,3′-difluoro-4′-glucuronide phenyl)-1,3-benzoxazol-5-sulfate; 7-(1-bromovinyl)-2-(2′,3′-difluoro-4′-sulfate phenyl)-1,3-benzoxazol-5-glucuronide; 7-(1-bromovinyl)-2-(2′,3′-difluoro-4′-sulfate phenyl)-1,3-benzoxazol-5-sulfate; 7-allyl-2-(3′-fluoro-4′-glucuronide phenyl)-1,3-benzoxazol-5-ol; 7-allyl-2-(3′-fluoro-4′-sulfate phenyl)-1,3-benzoxazol-5-ol; 7-allyl-2-(3′-fluoro-4′-hydroxyphenyl)-1,3-benzoxazol-5-glucuronide; 7-allyl-2-(3′-fluoro-4′-hydroxyphenyl)-1,3-benzoxazol-5-sulfate; 7-allyl-2-(3′-fluoro-4′-glucuronide phenyl)-1,3-benzoxazol-5-glucuronide; 7-allyl-2-(3′-fluoro-4′-glucuronide phenyl)-1,3-benzoxazol-5-sulfate; 7-allyl-2-(3′-fluoro-4′-sulfate phenyl)-1,3-benzoxazol-5-glucuronide; 7-allyl-2-(3′-fluoro-4′-sulfate phenyl)-1,3-benzoxazol-5-sulfate; 2-(3+, 5′-difluoro-4′-glucuronide phenyl)-7-vinyl-1,3-benzoxazol-5-ol; 2-(3′,5′-difluoro-4′-sulfate phenyl)-7-vinyl-1,3-benzoxazol-5-ol; 2-(3′,5′-difluoro-4′-hydroxy phenyl)-7-vinyl-1,3-benzoxazol-5-glucuronide; 2-(3′,5′-difluoro-4′-hydroxyphenyl)-7-vinyl-1,3-benzoxazol-5-sulfate; 2-(3′,5′-difluoro-4′-glucuronide phenyl)-7-vinyl-1,3-benzoxazol-5-g luguronide; 2-(3′,5′-difluoro-4′-glucuronide phenyl)-7-vinyl-1,3-benzoxazol-5-sulfate; 2-(3′,5′-difluoro-4′-sulfate phenyl)-7-vinyl-1,3-benzoxazol-5-glucuronide; 2-(3′,5′-difluoro-4′-sulfate phenyl)-7-vinyl-1,3-benzoxazol-5-sulfate; 2-(3′-fluoro-4′-glucuronide phenyl)-7-(1-fluorovinyl)-1,3-benzoxazol-5-ol; 2-(3′-fluoro-4′-sulfate phenyl)-7-(1-fluorovinyl)-1,3-benzoxazol-5-ol; 2-(3′-fluoro-4′-hydroxyphenyl)-7-(1-fluorovinyl)-1,3-benzoxazol-5-glucuronide; 2-(3′-fluoro-4′-hydroxyphenyl)-7-(1-fluorovinyl)-1,3-benzoxazol-5-sulfate; 2-(3′-fluoro-4′-glucuronide phenyl)-7-(1-fluorovinyl)-1,3-benzoxazol-5-glucuronide; 2-(3′-fluoro-4′-g lucuronide phenyl)-7-(1-fluorovinyl)-1,3-benzoxazol-5-sulfate; 2-(3′-fluoro-4′-sulfate phenyl)-7-(1-fluorovinyl)-1,3-benzoxazol-5-glucuronide; or 2-(3′-fluoro-4′-sulfate phenyl)-7-(1-fluorovinyl)-1,3-benzoxazol-5-sulfate. In further embodiments, the compound is glucuronide derivative, a sulfate derivative, or a glucuronide-sulfate derivative of 2-(5-hydroxy-1,3-benzoxazol-2-yl) benzene-1,4-diol; 3-(5-hydroxy-1,3-benzoxazol-2-yl)benzene-1,2-diol; 2-(3-fluoro-4-hydroxyphenyl)-1,3-benzoxazol-5-ol; 2-(3-chloro-4-hydroxyphenyl)-1,3-benzoxazol-5-ol; 2-(2-chloro-4-hydroxyphenyl)-1,3-benzoxazol-5-ol; 2-(3-fluoro-4-hydroxyphenyl)-1,3-benzoxazol-6-ol; 2-(3-tert-butyl-4-hydroxyphenyl)-1,3-benzoxazol-6-ol; 2-(6-hydroxy-1,3-benzoxazol-2-yl)benzene-1,4-diol; 3-(6-hydroxy-1,3-benzoxazol-2-yl)benzene-1,2-diol; 4-(6-hydroxy-1,3-benzoxazol-2-yl)benzene-1,2-diol; 2-(3-chloro-4-hydroxyphenyl)-1,3-benzoxazol-6-ol; 4-(5-hydroxy-1,3-benzoxazol-2-yl)benzene-1,3-diol; 4-(6-hydroxy-1,3-benzoxazol-2-yl)benzene-1,3-diol; 6-chloro-2-(3-fluoro-4-hydroxyphenyl)-1,3-benzoxazol-5-ol; 6-bromo-2-(3-fluoro-4-hydroxyphenyl)-1,3-benzoxazol-5-ol; 6-chloro-2-(4-hydroxyphenyl)-1,3-benzoxazol-5-ol; 5-chloro-2-(4-hydroxyphenyl)-1,3-benzoxazol-6-ol; 7-bromo-2-(3-fluoro-4-hydroxyphenyl)-1,3-benzoxazol-5-ol; 7-bromo-2-(2-fluoro-4-hydroxyphenyl)-1,3-benzoxazol-5-ol; 7-bromo-2-(2,3-difluoro-4-hydroxyphenyl)-1,3-benzoxazol-5-ol; 2-(4-hydroxyphenyl)-7-vinyl-1,3-benzoxazol-5-ol; 7-(1,2-dibromoethyl)-2-(4-hydroxyphenyl)-1,3-benzoxazol-5-ol; 7-(1-bromovinyl)-2-(4-hydroxyphenyl)-1,3-benzoxazol-5-ol; 7-ethynyl-2-(4-hydroxyphenyl)-1,3-benzoxazol-5-ol; 2-(4-hydroxyphenyl)-7-propyl-1,3-benzoxazol-5-ol; 7-butyl-2-(4-hydroxyphenyl)-1,3-benzoxazol-5-ol; 7-cyclopentyl-2-(4-hydroxyphenyl)-1,3-benzoxazol-5-ol; ethyl 5-hydroxy-2-(4-hydroxyphenyl)-1,3-benzoxazole-7-carboxylate; 2-(4-hydroxyphenyl)-7-phenyl-1,3-benzoxazol-5-ol; 2-(4-hydroxyphenyl)-7-methoxy-1,3-benzoxazol-5-ol; 7-ethyl-2-(4-hydroxyphenyl)-1,3-benzoxazol-5-ol; 7-ethyl-2-(2-ethyl-4-hydroxyphenyl)-1,3-benzoxazol-5-ol; 5-hydroxy-2-(4-hydroxyphenyl)-1,3-benzoxazole-7-carbaldehyde; 7-(hydroxymethyl)-2-(4-hydroxyphenyl)-1,3-benzoxazol-5-ol; 7-(bromomethyl)-2-(4-hydroxyphenyl)-1,3-benzoxazol-5-ol; [5-hydroxy-2-(4-hydroxyphenyl)-1,3-benzoxazol-7-yl] acetonitrile; 7-(1-hydroxy-1-methylethyl)-2-(4-hydroxyphenyl)-1,3-benzoxazol-5-ol]; 2-(4-hydroxyphenyl)-7-isopropenyl-1,3-benzoxazol-5-ol ; 2-(4-hydroxyphenyl)-7-isopropyl-1,3-benzoxazol-5-ol]; 7-bromo-2-(4-hydroxy-3-(trifluoromethyl)phenyl)-1,3-benzoxazol-5-ol; 7-(2-furyl)-2-(4-hydroxyphenyl)-1,3-benzoxazol-5-ol; 2-(3-fluoro-4-hydroxyphenyl)-7-(2-furyl)-1,3-benzoxazol-5-ol; 2-(4-hydroxyphenyl)-7-thien-2-yl-1,3-benzoxazol-5-ol; 2-(4-hydroxyphenyl)-7-(1,3-thiazol-2-yl)-1,3-benzoxazol-5-ol; 2-(3-fluoro-4-hydroxyphenyl)-5-hydroxy-1,3-benzoxazole-7-carbonitrile; 4-bromo-2-(4-hydroxyphenyl)-7-methoxy-1,3-benzoxazol-5-ol; 4,6-dibromo-2-(4-hydroxyphenyl)-7-methoxy-1,3-benzoxazol-5-ol; or 7-bromo-2-(3,5-difluoro-4-hydroxyphenyl)-1,3-benzoxazol-5-ol.

The present invention provides prodrug derivatives of substituted benzoxazoles, which are useful as estrogenic agents. In some embodiments, the compounds of the invention are derivatives that possess one or more appended sulfate (i.e., —O—S(═O)₂—O—H), unmodified or modified hexose (for example, glucuronide) or both. Suitable compounds that can be derivatized to form compounds of the present invention can be found in U.S. patent application Ser. No. 10/309,699 filed Dec. 4, 2002, which is incorporated herein by reference in its entirety.

As used herein, the term “hexose” means a sugar containing six carbon atoms. Suitable hexoses include but are not limited to glucose, mannose, galactose and fructose, in both their straight chain and pyranose forms. Modified hexoses include naturally occurring derivatives of hexoses, for example, phosphates, and corresponding acid and lactone forms. For example, the term “modified hexose” includes gluconic acid, gluconolactone, glucuronic acid, amino derivates including N-acetyl derivatives, phosphoate derivatives, and the like.

As used herein, the term “glucuronide derivative,” as applied to a specific compound, refers to a derivative of such compound where one or more hydroxyl groups of the compound have been replaced with a moiety of formula XX:

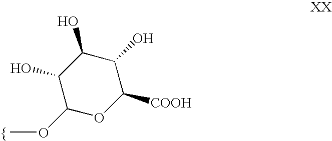

As used herein, the term “sulfate derivative,” as applied to a specific compound, refers to a derivative of such compound where one or more hydroxyl groups of the compound have been replaced with a moiety of formula —O—S(═O)₂—O—H.

The term “glucuronide-sulfate derivative,” as applied to a specific compound, refers to a derivative of such compound where at least one hydroxyl group of the compound has been replaced with a moiety of formula XX, and at least one hydroxyl group of the compound has been replaced with a moiety of formula O—S(═O)₂—O—H.

The compounds of the present invention are substituted benzoxazole estrogenic agents, which have been derivatized to possess one or more appended moieties. After administration of the derivatized compound, the appended moieties are removed by endogenous enzymes to provide the underivatized compound. Such compounds are referred to here as metabolites of the compounds of the invention.

As used in accordance with this invention, the term “providing,” with respect to providing a compound or substance covered by this invention, means either directly administering such a compound or substance, or administering a prodrug, derivative, or analog that will form the effective amount of the compound or substance within the body.

As used in accordance with this invention, the term “ER□ selective ligand” means that the binding affinity (as measured by IC₅₀, where the IC₅₀ of 17β-estradiol is not more than 3 fold different between ERα and ERβ) of the ligand to ER□ is at least about 10 times greater than its binding affinity to ER□ in a standard pharmacological test procedure that measures the binding affinities to ER□ and ER□. It is preferred that the ER□ selective ligand will have a binding affinity to ER□ that is at least about 20 times greater than its binding affinity to ER□. It is more preferred that the ER□ selective ligand will have a binding affinity to ER□ that is at least about 50 times greater than its binding affinity to ER□. It is further preferred that the ER□ selective ligand is non-uterotrophic and non-mammotrophic.

As used in accordance with this invention, the term “non-uterotrophic” means producing an increase in wet uterine weight in a standard pharmacological test procedure of less than about 50% of the uterine weight increase observed for a maximally efficacious dose of 17β-estradiol or 17α-ethinyl-17β-estradiol in the same procedure. It is preferred that the increase in wet uterine weight will be less than about 25% of that observed for estradiol, and more preferred that the increase in wet uterine weight will be less than about 10% of that observed for estradiol. It is most preferred that the non-uterotrophic ER□ selective ligand will not increase wet uterine weight significantly (p>0.05) compared with a control that is devoid of uterotrophic activity (e.g., vehicle).

As used in accordance with this invention, the term “non-mammotrophic” means having activity that is <10% as efficacious as 17beta-estradiol at facilitating the development of lobular-alveolar end buds as assessed by histological examination. Examples of such determination by histological examination are well known in the art. See, for example, Harris, H. A., et al., Endocrinology 144(10) 4241-4249 (2003); Mulac-Jericevic, B., et al., Proc. Natl. Acad. Sci. 100 (17) 9744-9749 (2003); Bocchinfuso, W. P., et al., Endocrinology 141(8) 2982-2994 (2002); and Lewis, B. C., et al., Toxicological Sciences 62, 46-53 (2001), each of which is incorporated by reference herein in its entirety.

This invention also provides the use of the disclosed derivatized ER□ selective ligands in the treatment or inhibition of arthritis, inflammatory bowel disease, and endometriosis. More particularly, the derivatized ER□ selective ligands are useful in the treatment or inhibition of rheumatoid arthritis, osteoarthritis or spondyloarthropathies; and Crohn's disease, ulcerative colitis, indeterminate colitis, infectious colitis, or ulcerative proctitis. This invention further provides for the use of a derivatized ER□ selective ligand in treating or inhibiting joint swelling or erosion; or treating or inhibiting joint damage secondary to arthroscopic or surgical procedures. It is preferred that the ER□ selective ligand is non-uterotrophic and non-mammotrophic.

The present invention also provides the disclosed derivatized ER□ selective ligands for use in lowering cholesterol, triglycerides, Lp(a), or LDL levels; inhibiting or treating hypercholesteremia, hyperlipidemia, cardiovascular disease, atherosclerosis, hypertension, peripheral vascular disease, restenosis, or vasospasm; or inhibiting vascular wall damage from cellular events leading toward immune mediated vascular damage in a mammal in need thereof.

Further, the disclosed derivatized ER□ selective ligands are useful in providing cognition enhancement or neuroprotection; or treating or inhibiting senile dementias, Alzheimer's disease, cognitive decline, stroke, anxiety, or neurodegenerative disorders in a mammal in need thereof.

The invention further provides the use of the disclosed ER□ ligands for the treatment and inhibition of free radical induced disease states, vaginal or vulvar atrophy, atrophic vaginitis, vaginal dryness, pruritus, dyspareunia, dysuria, frequent urination, urinary incontinence, urinary tract infections, vasomotor symptoms, psoriasis or dermatitis, ischemia, reperfusion injury, asthma, pleurisy, multiple sclerosis, systemic lupus erythematosis, uveitis, sepsis, hemmorhagic shock, or type II diabetes, in a mammal in need thereof.

The ER□ selective ligands of the present invention of formula I are also useful in inhibiting conception in a mammal in need thereof.

In some embodiments, the mammal is a human, e.g., a woman.

The present invention further provides a pharmaceutical composition comprising a compound of formula I, as described hereinbefore, and a pharmaceutical carrier.

The reagents used in the preparation of the compounds of this invention can be either commercially obtained or can be prepared by standard procedures described in the literature.

The general preparation of compounds of formula I that can be derivatized by the addition of one or more moieties selected from sulfate and modified and unmodified hexoses, can be prepared according to the following synthetic Schemes (I-VIII).

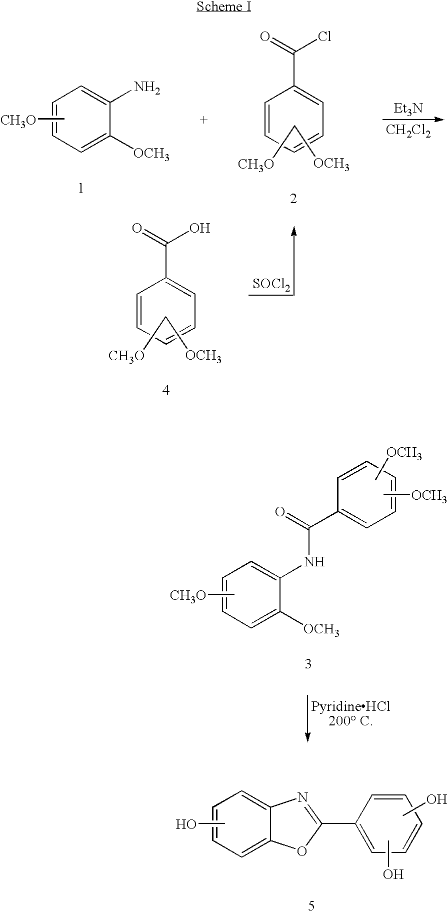

In Scheme I, commercially available dimethoxy aniline (1) was treated with commercially available benzoyl chloride (2) in the presence of triethylamine to produce an amide (3). The required benzoyl chloride (2) was also prepared from commercially available benzoic acid (4) upon refluxing with thionyl chloride. The amide (3) was converted to the phenolic benzoxazole (5) upon treatment with pyridine hydrochloride at high temperature (200° C.).

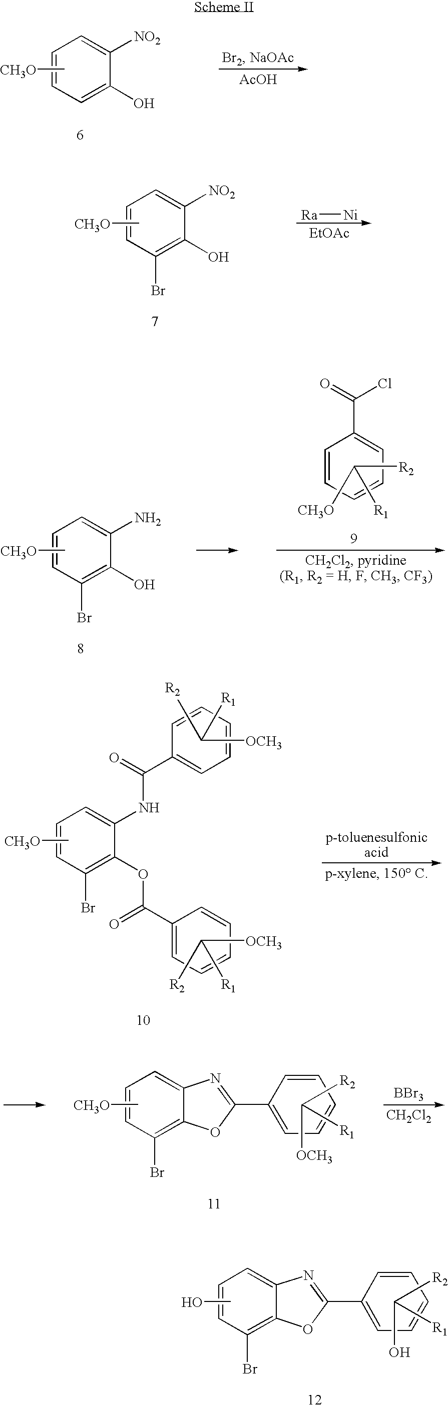

In Scheme II, commercially available nitro-phenol (6) was brominated with Br₂/NaOAc in acetic acid to produce bromo-phenol (7). Catalytic hydrogenation of (7) with Ra—Ni in EtOAc afforded aniline (8). Coupling of (8) with benzoyl chloride (9) (commercially available, or prepared from the corresponding benzoic acid and thionyl chloride) in the presence of pyridine produced amide-ester (10). Conversion of (10) to benzoxazole (11) was accomplished under acidic conditions (p-toluenesulfonic acid) at high temperature (150° C.). Demethylation of (11) with boron tribromide in dichloromethane afforded the phenolic benzoxazole (12).

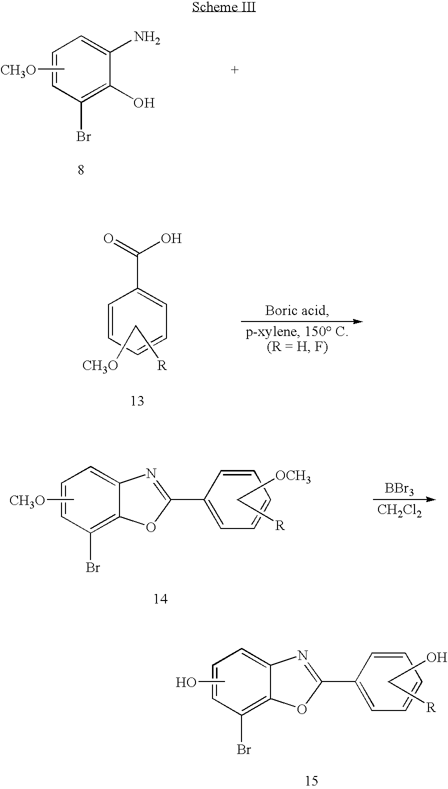

In Scheme III, the aniline (8) was converted to benzoxazole (14) upon treatment with benzoic acid (13) and boric acid in p-xylene at high temperature (150° C.). Demethylation of (14) with boron tribromide in dichloromethane produced the phenolic benzoxazole (15).

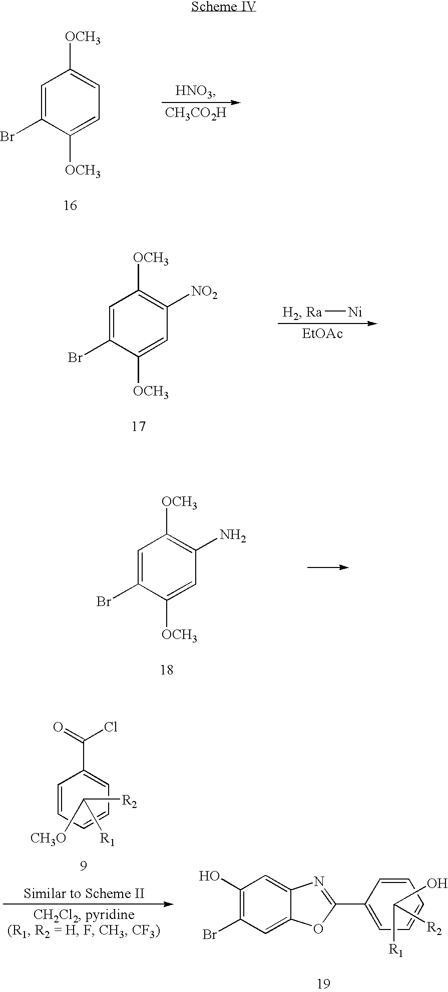

In Scheme IV, nitration of (16) with nitric acid in acetic acid produced (17), which was reduced with hydrogen in the presence of Ra—Ni to afford aniline (18). Aniline (18) was converted to benzoxazole (19) in a similar manner as described in Scheme II, with the exemption that the demethylation step was accomplished with pyridine hydrochloride at high temperature (200° C.).

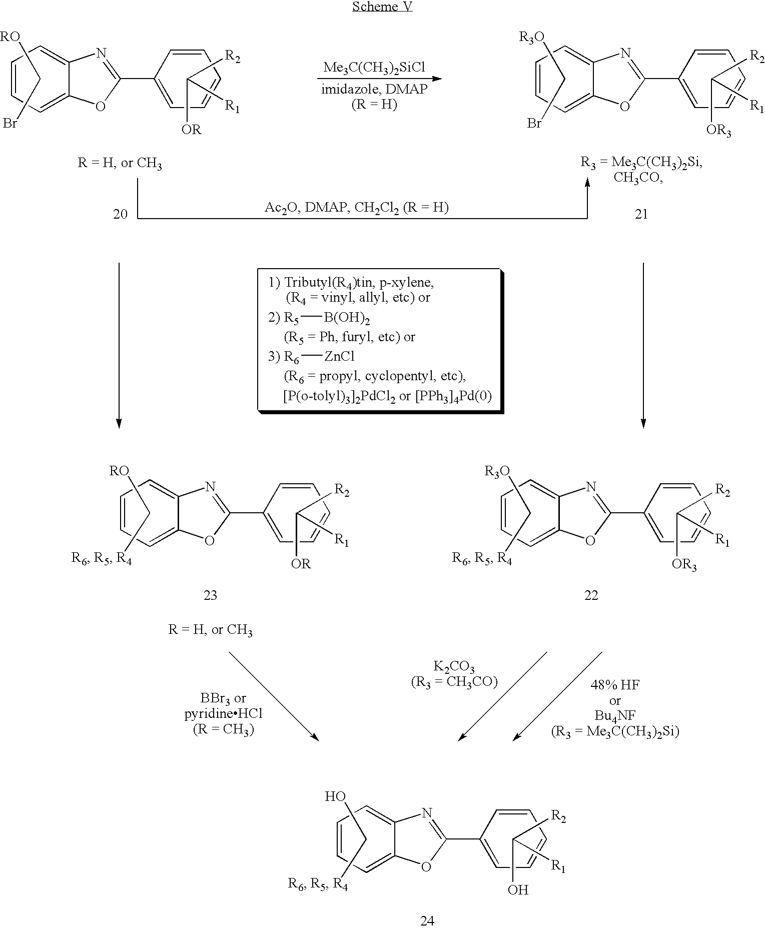

In Scheme V, the hydroxyl groups of benzoxazole (20) were protected either as the silyl ethers (21) (R₃=Me₃C(CH₃)₂Si) with tert-butyidimethylsilyl chloride/imidazole/4-dimethylaminopyridine in N,N-dimethylformamide, or as the esters (21) (R₃═CH₃CO) with acetic anhydride/4-dimethylaminopyridine in dichloromethane. Benzoxazoles (20) and (21) were coupled with a variety of tin reagents (i.e., tributyl(vinyl)tin, tributyl(allyl)tin, tributyl(2-furyl)tin, boronic acids or zinc chlorides in the presence of a palladium catalyst [i.e., dichlorobis(tri-o-tolylphosphine)palladium(lI) or tetrakis(triphenylphosphine) palladium(0)] in p-xylene, toluene, tetrahydrofuran, dimethoxymethane or 1,2-dimethoxyethane, with the presence of a base (i.e., Na₂CO₃) for the boronic acid coupling reaction, at temperatures in the range of 20° C. to 150° C., to produce benzoxazoles (22) and (23).

Deprotection of the silyl ethers of (22) (R₃=Me₃C(CH₃)₂Si) with hydrofluoric acid (48 wt. % in water) or tetrabutylammonium fluoride produced benzoxazole (24). Saponification of (22) (R₃═CH₃CO) with potassium carbonate in dioxane produced benzoxazole (24). Benzoxazole 23 (R═CH₃) was demethylated with boron tribromide in dichloromethane or pyridine hydrochloride at high temperature (200° C.) to afford benzoxazole (24).

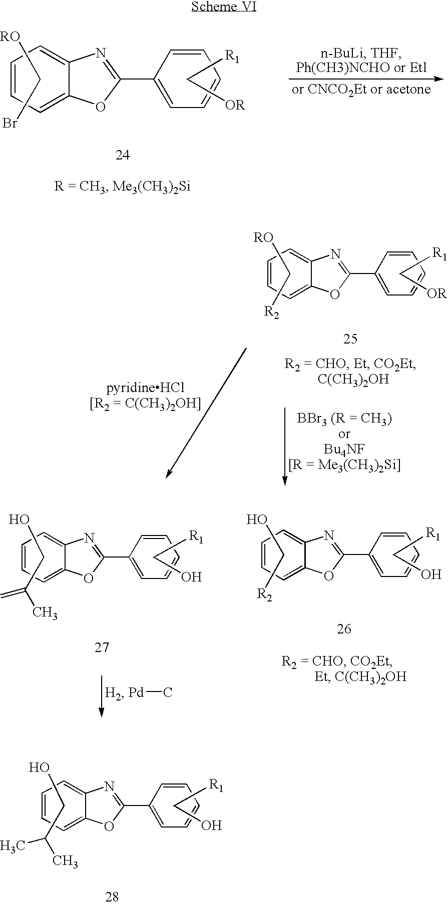

In Scheme VI, benzoxazole (24) was treated with n-butyllithium at low temperatures (−78° C.) followed by the addition of an electrophile (i.e. CNCO₂Et, Ph(CH₃₎NCHO, Etl, etc.) to produce compound (25). Deprotection of (25) with boron tribromide (R═CH₃) or tetrabutylammonium fluoride (R=Me₃C(CH₃)₂Si) afforded benzoxazole (26) [R═CHO, CO₂Et, CH₂CH₃, C(CH₃)₂OH].

The tertiary alcohol (25) (R═C(CH₃)OH) was treated with pyridine hydrochloride at high temperature (200° C.) to produce 1-methyl-vinyl benzoxazole (27). Reduction of (27) with H₂ /Pd—C afforded the isopropyl analog (28).

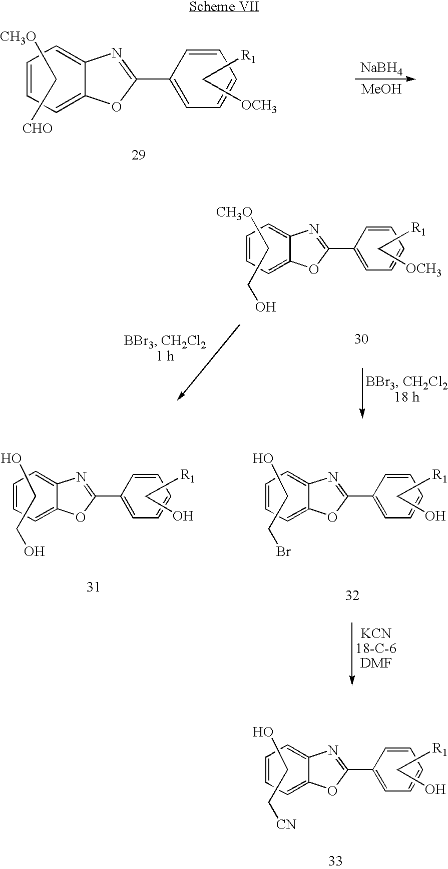

In Scheme VII, reduction of the benzoxazole (29) with sodium borohydride in methanol produced alcohol (30). Treatment of (30) with boron tribromide in CH₂Cl₂ for 1 hour furnished benzoxazole (31), while prolonged (18 hours) treatment afforded bromide (32). Bromide (32) was converted to acetonitrile (33) upon treatment with potassium cyanide and 18-crown-6 ether in N,N-dimethylformamide.

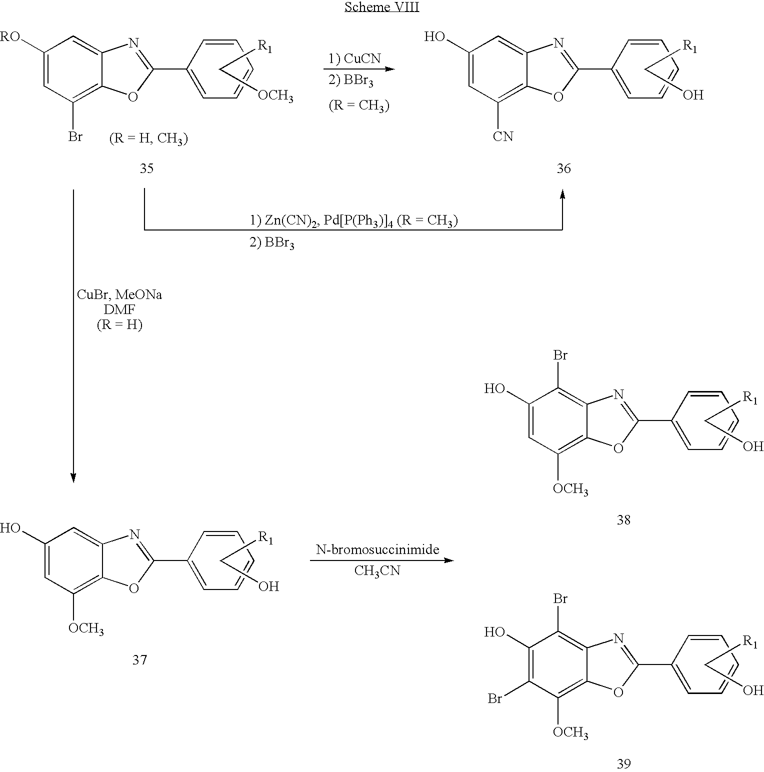

In Scheme VIII, bromo-benzoxazole (35) (R═CH₃) was first treated with copper(I) cyanide in DMF to produce the corresponding aryl-nitrile, which upon treatment with boron tribromide afforded benzoxazole (36). Benzoxazole (36) was also prepared from a second synthetic Route, where the bromo-benzoxazole (35) was treated with zinc cyanide in the presence of a palladium catalyst [i.e. tetrakis(triphenylphosphine)palladium(0)] to afford the corresponding aryl-nitrile, which upon demethylation with boron tribromide produced benzoxazole (36). Benzoxazole (35) (R═H) was treated with copper (I) bromide, and freshly prepared sodium methoxide in DMF to produce methoxy-benzoxazole (37). Bromination of (37) with N-bromosuccinimide in acetonitrile afforded the monobromo benzoxazole (38) (major product) and the dibromobenzoxazole (39) (minor product).

Glucuronide, sulfate, and glucuronide-sulfate derivatives of the compounds prepared by the procedures of Schemes I-VIII can be prepared according to Schemes IX and X:

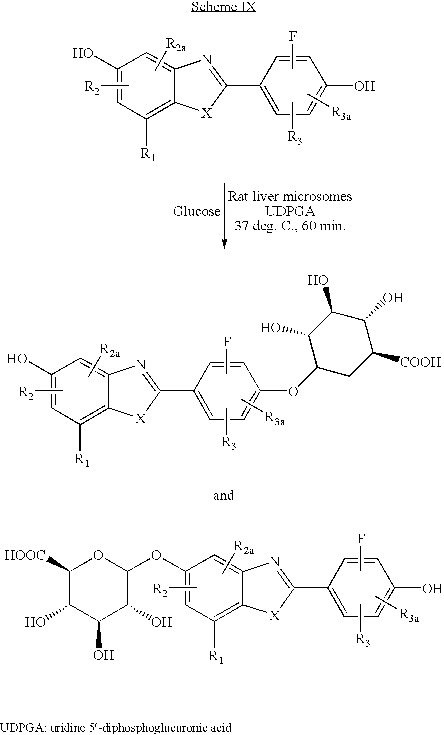

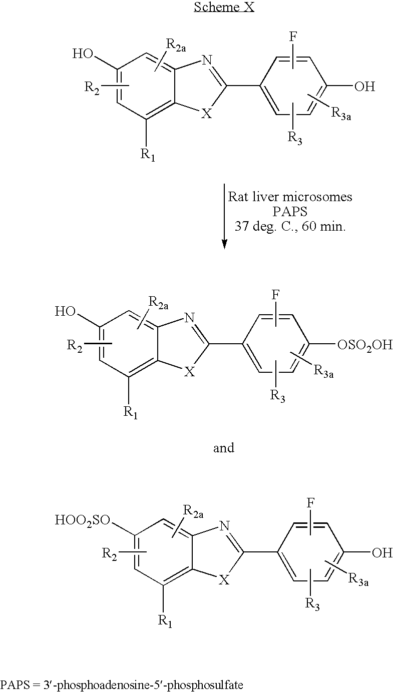

In addition, the glucuronide, sulfate and glucuronide-sulfate derivatives of the invention can be prepared according to standard organic chemical synthetic techniques. For example, functional groups (e.g., one or more hydroxyl groups) of compounds prepared in accordance with Schemes I-VIII can be protected by standard techniques, and then a free hydroxyl can be coupled to a unmodified or modified hexose (e.g., a glucuronide) or a sulfonic acid group, to yield a compound of the invention. Suitable protecting groups for use in such syntheses can be found in, for example, Greene, T. W., and Wuts, P. G. M., Protective Groups in Organic Synthesis, 2^{nd} ed., New York: John Wiley & Sons, N.Y. 1991.

Pharmacological Test Procedures

Standard pharmacological test procedures are readily available to determine the activity profile of a given test compound. The following briefly summarizes several representative test procedures and may include data for representative compounds of the invention. All assays, except the radioligand binding assay, can be used to detect estrogen receptor agonist or antagonist activity of compounds. In general, estrogen receptor agonist activity is measured by comparing the activity of the compound to a reference estrogen (e.g., 17β-estradiol, 17α-ethinyl, 17β-estradiol, estrone, diethylstilbesterol, etc). Estrogen receptor antagonist activity is generally measured by co-treating the test compound with the reference estrogen and comparing the result to that obtained with the reference estrogen alone. Standard pharmacological test procedures for SERMs are also provided in U.S. Pat. Nos. 4,418,068 and 5,998,402, which are hereby incorporated by reference.

Evaluation of Binding Affinities to ERα and ERβ

Representative examples of metabolites of compounds of the invention were evaluated for their ability to compete with 17β-estradiol for both ERα and ERβ in a conventional radioligand binding assay. This test procedure provides the methodology for one to determine the relative binding affinities for the ERα or ERβ receptors. The procedure used is briefly described below.

Preparation of receptor extracts for characterization of binding selectivity. The ligand binding domains, conveniently defined here as all sequence downstream of the DNA binding domain, were obtained by PCR using full length cDNA as templates and primers that contained appropriate restriction sites for subcloning while maintaining the appropriate reading frame for expression. These templates contained amino acids M₂₅₀-V₅₉₅ of human ERα [Green, et al., Nature 320: 134-9 (1986)] and M₂₁₄-Q₅₃₀ of human ERα [Ogawa, et al., Biochemical & Biophysical Research Communications 243: 122-6 (1998)]. Human ERβ was cloned into pET15b (Novagen, Madison, Wis.) as a Nco1-BamH1 fragment bearing a C-terminal Flag tag. Human ERα was cloned as for human ERβ except that an N-terminal His tag was added. The sequences of all constructs used were verified by complete sequencing of both strands.

BL21 (DE3) cells were used to express the human proteins. Typically, a 10 mL overnight culture was used to inoculate a 1 L culture of Luria-Bertani (LB) medium containing 100 μg/mL of ampicillin. After incubation overnight at 37° C., isopropyl-β-D-thiogulactoside (IPTG) was added to a final concentration of 1 mM and incubation proceeded at 25° C. for 2 hours. Cells were harvested by centrifugation (1500×g), and the pellets washed with and resuspended in 100 mL of 50 mM Tris-Cl (pH 7.4) and 150 mM NaCl. Cells were lysed by passing twice through a French press at 12000 psi. The lysate was clarified by centrifugation at 12,000×g for 30 minutes at 4° C. and stored at −70° C.

Evaluation of extracts for specific [³H]-estradiol binding. Dulbecco's phosphate buffered saline (1× final concentration Gibco®; nitrogen, Carlsbad, Calif.) supplemented with 1 mM ethylenediamine-tetraacetic acid (EDTA) was used as the assay buffer. To optimize the amount of receptor to use in the assay, [³H]-17β-estradiol (final concentration =2 nM; New England Nuclear (NEN); Perkin Elmer, Shelton, Conn.) ±0.6 μM diethlystilbestrol and 100 μL of various dilutions of the E. coli lysate were added to each well of a high binding masked microtiter plate (EG&G Wallac). The final assay volume was 120 μL and the concentration of DMSO was ≦1%. After incubation at room temperature for 5-18 hours, unbound material was aspirated and the plate washed three times with approximately 300 μL of assay buffer. After washing, 135 μL of scintillation cocktail (Optiphase Supermix, EG&G Wallac) was added to the wells, and the plate was sealed and agitated for at least 5 minutes to mix scintillant with residual wash buffer. Bound radioactivity was evaluated by liquid scintillation counting (Plus EG&G Wallac, Microbeta).

After determining the dilution of each receptor preparation that provided maximum specific binding, the assay was further optimized by estimating the IC₅₀ of unlabelled 17β-estradiol using various dilutions of the receptor preparation. A final working dilution for each receptor preparation was chosen for which the IC₅₀ of unlabelled 17β-estradiol was 2-4 nM.

Ligand binding competition test procedure. Test compounds were initially solubilized in dimethylsulfoxide (DMSO) and the final concentration of DMSO in the binding assay was ≦1%. Eight dilutions of each test compound were used as an unlabelled competitor for [³H]-17β-estradiol. Typically, a set of compound dilutions were tested simultaneously on human ERα and ERβ. The results were plotted as measured distinguished per minute (DPM) vs. concentration of test compound. For dose-response curve fitting, a four parameter logistic model on the transformed, weighted data was fitted and the IC₅₀ was defined as the concentration of compound that decreased maximum [³H]-estradiol binding by 50%.

Binding affinities for ERα and ERβ (as measured by IC₅₀) for representative metabolites of compounds of the invention are shown in Table 1.

TABLE 1
ER Binding Affinities of Representative Metabolites of Compounds
of the Invention

| | ER-β IC₅₀ (nM) | ER-α IC₅₀ (nM) |
|---|---|---|
| 1 | 140 | 720 |
| 2 | 963 | 5110 |
| 3 | 66 | 1570 |
| 4 | 239 | 5280 |
| 5 | 59 | 139 |
| 6 | 39 | 843 |
| 7 | 1600 | 5000 |
| 8 | 181 | 2353 |
| 9 | 440 | 1500 |
| 10 | 105 | 2040 |
| 11 | 703 | >5000 |
| 12 | 49 | 1227 |
| 13 | 25 | 190 |
| 14 | 50 | 902 |
| 15 | 3 | 82 |
| 16 | 64 | 1813 |
| 17 | 42 | 1210 |
| 18 | 16 | 464 |
| 19 | 157 | 2765 |
| 20 | 2 | 155 |
| 21 | 3 | 260 |
| 22 | 1 | 47 |
| 23 | 3 | 113 |
| 24 | 6 | 1217 |
| 25 | 2 | 227 |
| 26 | 4 | 474 |
| 27 | 4 | 409 |
| 28 | 25 | 1036 |
| 29 | 155 | 803 |
| 30 | 134 | 3080 |
| 31 | 31 | 352 |
| 32 | 16 | 196 |
| 33 | 31 | 352 |
| 34 | 14 | 1101 |
| 35 | 15 | 481 |
| 36 | 11 | 390 |
| 37 | 79 | 498 |
| 38 | 102 | 1010 |
| 39 | 190 | 7827 |
| 40 | 235 | 1300 |
| 41 | 6 | 411 |
| 42 | 95 | 9620 |
| 43 | 59 | 2557 |
| 44 | 13 | 537 |
| 45 | 84 | 655 |
| 46 | 59 | 2638 |
| 47 | 1340 | Not determined |
| 48 | 40 | 2975 |
| 49 | 1042 | 5230 |
| 50 | 399 | >5000 |
| 51 | 142 | 775 |
| 52 | 82 | 1200 |
| 53 | 166 | 1870 |
| 54 | 135 | 809 |
| 55 | 313 | 1980 |
| 56 | 97 | 1030 |
| 57 | 366 | 1340 |
| 58 | 26 | 1435 |
| 59 | 52 | 2668 |
| 60 | 64 | 559 |
| 61 | 93 | 1180 |
| 62 | 201 | >10000 |
| 63 | 1 | 44 |
| 64 | 3 | 376 |

The results obtained in the standard pharmacologic test procedure described above demonstrate that the tested compounds bind both subtypes of the estrogen receptor. The IC₅₀s are generally lower for ERβ, indicating that these compounds are preferentially ERβ selective ligands, but are still considered active at ERα. The compounds will exhibit a range of activity based, at least partially, on their receptor affinity selectivity profiles. Since the metabolites of the compounds of the invention bind ERβ with higher affinity than ERα, the compounds of the invention will be useful in treating or inhibiting diseases than can be modulated via ERβ. Additionally, since each receptor ligand complex is unique and thus, its interaction with various coregulatory proteins is unique, the compounds of this invention will display different and unpredictable activities depending on cellular context. For example, in some cell types, it is possible for a compound to behave as an estrogen receptor agonist while in other tissues, as an estrogen receptor antagonist. Compounds with such activity have sometimes been referred to as SERMs (Selective Estrogen Receptor Modulators). Unlike many estrogens, however, many of the SERMs do not cause increases in uterine wet weight. These compounds are antiestrogenic in the uterus and can completely antagonize the trophic effects of estrogen receptor agonists in uterine tissue. These compounds, however, act as estrogen receptor agonists in the bone, cardiovascular, and central nervous systems. Due to this tissue selective nature of these compounds, they are useful in treating or inhibiting in a mammal disease states or syndromes that are caused or associated with an estrogen deficiency (in certain tissues such as bone or cardiovascular) or an excess of estrogen (in the uterus or mammary glands). In addition, metabolites of compounds of this invention have the potential to behave as estrogen receptor agonists on one receptor type while behaving as estrogen receptor antagonists on the other. For example, it has been demonstrated that compounds can antagonize the action of 17β-estradiol via ERβ while exhibiting estrogen receptor agonist activity with ERα [Sun, et al., Endocrinology 140: 800-804 (1999)]. Such ERSAA (Estrogen Receptor Selective Agonist Antagonist) activity provides for pharmacologically distinct estrogenic activity within this series of compounds

Regulation of Metallothionein II mRNA

Estrogens acting through ERβ, but not ERα, can upregulate metallothionein II mRNA levels in Saos-2 cells, as described by Harris et al. [Endocrinology 142(2): 645-652 (2001)]. Results from this test procedure can be combined with results from the test procedure described below (ERE reporter test procedure) to generate a selectivity profile for metabolites of compounds of this invention (see also, WO 00/37681). Data for representative metabolites of compounds of the invention are shown in Table 2.

TABLE 2
Regulation of Metallothionein-II mRNA in Saos-2 Cells

| Compound | Fold regulation |
|---|---|
| Example 12 | 9.6 |
| Example 14 | 12.4 |
| Example 13 | 9.7 |

Evaluation of Test Compound Using an ERE-Reporter Test Procedure in MCF-7 Breast Cancer Cells

Stock solutions of test compounds (usually 0.1 M) are prepared in DMSO and then diluted 10 to 100-fold with DMSO to make working solutions of 1 or 10 mM. The DMSO stocks are stored at either 4° C. (0.1 M) or −20° C. (<0.1 M). MCF-7 cells are passaged twice a week with growth medium [D-MEM/F-12 medium containing 10% (v/v) heat-inactivated fetal bovine serum, 1% (v/v) Penicillin-Streptomycin, and 2 mM glut,aMax-1]. The cells are maintained in vented flasks at 37° C. inside a 5% CO₂/95% humidified air incubator. One day prior to treatment, the cells are plated with growth medium at 25,000 cells/well into 96 well plates and incubated at 37° C. overnight.

The cells are infected for 2 hours at 37° C. with 50 μl/well of a 1:10 dilution of adenovirus 5-ERE-tk-luciferase in experimental medium [phenol red-free D-MEM/F-12 medium containing 10% (v/v) heat-inactived charcoal-stripped fetal bovine serum, 1% (v/v) Penicillin-Streptomycin, 2 mM glutaMax-1, and 1 mM sodium pyruvate]. The wells are then washed once with 150 μl of experimental medium. Finally, the cells are treated for 24 hours at 37° C. in replicates of 8 wells/treatment with 150 μl/well of vehicle (≦0.1% v/v DMSO) or test compound that is diluted ≧1000-fold into experimental medium.

Initial screening of test compounds is done at a single dose of 1 μM that is tested alone (estrogen receptor agonist mode) or in combination with 0.1 nM 17β-estradiol (EC₈₀; estrogen receptor antagonist mode). Each 96 well plate also includes a vehicle control group (0.1% v/v DMSO) and an estrogen receptor agonist control group (either 0.1 or 1 nM 17β-estradiol). Dose-response experiments are performed in either the estrogen receptor agonist and/or estrogen receptor antagonist modes on active compounds in log increases from 10⁻¹⁴ to 10⁻⁵ M. From these dose-response curves, EC₅₀ and IC₅₀ values, respectively, are generated. The final well in each treatment group contains 5 μl of 3×10⁻⁵ M ICI-182,780 (10⁻⁶ M final concentration) as an estrogen receptor antagonist control.

After treatment, the cells are lysed on a shaker for 15 minutes with 25 μl/well of 1× cell culture lysis reagent (Promega Corporation, Madison, Wis.). The cell lysates (20 μl) are transferred to a 96 well luminometer plate, and luciferase activity is measured in a MicroLumat LB 96 P luminometer (EG & G Berthold; Perkin Elmer, Shelton, Conn.) using 100 μl/well of luciferase substrate (Promega Corporation). Prior to the injection of substrate, a 1 second background measurement is made for each well. Following the injection of substrate, luciferase activity is measured for 10 seconds after a 1 second delay. The data are transferred from the luminometer to a Macintosh personal computer and analyzed using the JMP software (SAS Institute, Cary, N.C.); this program subtracts the background reading from the luciferase measurement for each well and then determines the mean and standard deviation of each treatment.

The luciferase data are transformed by logarithms, and the Huber M-estimator is used to down-weight the outlying transformed observations. The JMP software is used to analyze the transformed and weighted data for one-way ANOVA (Dunnett's test). The compound treatments are compared to the vehicle control results in the estrogen receptor agonist mode, or the positive estrogen receptor agonist control results (0.1 nM 17β-estradiol) in the estrogen receptor antagonist mode. For the initial single dose experiment, if the compound treatment results are significantly different from the appropriate control (p<0.05), then the results are reported as the percent relative to the 17β-estradiol control [i.e., ((compound−vehicle control)/(17β-estradiol control−vehicle control))×100]. The JMP software is also used to determine the EC₅₀ and/or IC₅₀ values from the non-linear dose-response curves.

Evaluation of Uterotrophic Activity

Uterotrophic activity of a test compound can be measured according to the following standard pharmacological test procedures.

Procedure 1: Sexually immature (18 days of age) Sprague-Dawley rats are obtained from Taconic (Germantown, N.Y.) and provided unrestricted access to a casein-based diet (Purina Mills® 5K96C, Purina Mills, LLC, St. Louis, Mo.) and water. On day 19, 20 and 21, the rats are dosed subcutaneously with 17α-ethinyl-17β-estradiol (0.06 μg/rat/day), test compound or vehicle (50% DMSO/50% Dulbecco's PBS). To assess estrogen receptor antagonist activity, compounds are coadministered with 17α-ethinyl-17β-estradiol (0.06 μg/rat/day). There are six rats/group and they are euthanized approximately 24 hours after the last injection by CO₂ asphyxiation and pneumothorax. Uteri are removed and weighed after trimming associated fat and expressing any internal fluid. A tissue sample can also be snap frozen for analysis of gene expression (e.g., complement factor 3 mRNA). Results obtained from representative metabolites of compounds of the invention are shown in Table 3.

TABLE 3
Evaluation of Select Compounds in a Rat Uterotrophic Test Procedure

| | mean uterine weight |
|---|---|
| Compound | (mg) ± SEM |
| Vehicle | 21.4 ± 1.59 |
| 17α-ethinyl, 17β-estradiol (0.06 μg/rat) | 85.5 ± 3.1 |
| Example 12 (2 mg/rat) + 17α-ethinyl, 17β- | 60.2 ± 4.0 |
| estradiol (0.06 μg/rat) | |
| Example 41 (2 mg/rat) | 30.3 ± 1.5 |
| Example 41 (2 mg/rat) + 17α-ethinyl, 17β- | 76.6 ± 3.0 |
| estradiol (0.06 μg/rat) | |
| Example 24 (2 mg/rat) | 14.18 ± 1.1 |
| Example 24 (2 mg/rat) + 17α-ethinyl, 17β- | 80.7 ± 5.3 |
| estradiol (0.06 μg/rat) | |
| Vehicle | 30.5 ± 3.2 |
| 17α-ethinyl, 17β-estradiol (0.06 μg/rat) | 104.7 ± 5.4 |
| Example 20 (2 mg/rat) | 39.2 ± 0.7 |
| Example 20 (2 mg/rat) + 17α-ethinyl, 17β- | 95.9 ± 5.5 |
| estradiol (0.06 μg/rat) | |
| Example 21 (2 mg/rat) | 38.8 ± 1.7 |
| Example 21 (2 mg/rat) + 17α-ethinyl, 17β- | 93.9 ± 5.9 |
| estradiol (0.06 μg/rat) | |

Procedure 2: Sexually immature (18 days of age) 129 SvE mice are obtained from Taconic and provided unrestricted access to a casein-based diet (Purina Mills® 5K96C) and water. On day 22, 23, 24 and 25, the mice are dosed subcutaneously with compound or vehicle (corn oil). There are six mice/group and they are euthanized approximately 6 hours after the last injection by CO₂ asphyxiation and pneumothorax. Uteri are removed and weighed after trimming associated fat and expressing any internal fluid. The following results (Table 4) were obtained for representative metabolites of compounds from the invention.

TABLE 4
Evaluation of Select Compounds in a Mouse Uterotrophic Test Procedure

| | mean uterine weight |
|---|---|
| Compound | (mg) ± SEM |
| vehicle | 10.2 ± 2.1 |
| 17β-estradiol (50 mg/kg) | 41.7 ± 3.6 |
| Example 21 (20 mg/kg) | 12.1 ± 1.7 |
| Vehicle | 11.7 ± 0.5 |
| 17β-estradiol (50 mg/kg) | 41.9 ± 2.9 |
| Example 24 (50 mg/kg) | 10.7 ± 0.9 |
| Vehicle | 9.6 ± 0.4 |
| 17β-estradiol (50 mg/kg) | 40.0 ± 2.0 |
| Example 34 (50 mg/kg) | 10.3 ± 0.7 |
| Vehicle | 9.4 ± 0.4 |
| 17β-estradiol (50 mg/kg) | 35.6 ± 4.4 |
| Example 25 (50 mg/kg) | 9.7 ± 1.0 |
| Vehicle | 13.7 ± 2.0 |
| 17β-estradiol (50 mg/kg) | 40.5 ± 5.84 |
| Example 12 (50 mg/kg) | 13.7 ± 0.82 |
| Example 20 (50 mg/kg) | 13.1 ± 0.86 |
| Vehicle | 9.6 ± 0.36 |
| 17β-estradiol (50 mg/kg) | 40.0 ± 2.0 |
| Example 34 (50 mg/kg) | 10.3 ± 0.69 |
| Vehicle | 9.8 ± 1.2 |
| 17β-estradiol (50 mg/kg) | 42.9 ± 4.8 |
| Example 26 (50 mg/kg) | 9.0 ± 0.3 |
| Example 42 (50 mg/kg) | 9.5 ± 0.6 |
| Example 64 (50 mg/kg) | 9.8 ± 0.7 |

Evaluation of Osteoporosis and Lipid Modulation (Cardioprotection)

Female Sprague-Dawley rats, ovariectomized or sham operated, are obtained 1 day after surgery from Taconic (weight range 240-275 g). They are housed 3 or 4 rats/cage in a room on a 12/12 (light/dark) schedule and provided with food (Purina Mills® 5K96C) and water ad libitum. Treatment for all studies begin 1 day after arrival and rats are dosed 7 days per week as indicated for 6 weeks. A group of age matched sham operated rats not receiving any treatment serve as an intact, estrogen replete control group for each study.

All test compounds are prepared in a vehicle of 50% DMSO (JT Baker, Phillipsburg, N.J.)/1× Dulbecco's phosphate saline (Gibco BRL, Grand Island, N.Y.) at defined concentrations so that the treatment volume is 0.1 mL/100 g body weight. 17β-estradiol is dissolved in corn oil (20 μg/mL) and delivered subcutaneously, 0.1 mL/rat. All dosages are adjusted at three week intervals according to group mean body weight measurements, and given subcutaneously.

Five weeks after the initiation of treatment and one week prior to the termination of the study, each rat is evaluated for bone mineral density (BMD). The total and trabecular density of the proximal tibia are evaluated in anesthetized rats using an XCT-960M peripheral quantitative computerized tomography (pQCT); Stratec Medizintechnik, Pforzheim, Germany). The measurements are performed as follows: Fifteen minutes prior to scanning, each rat is anesthetized with an intraperitoneal injection of 45 mg/kg ketamine, 8.5 mg/kg xylazine, and 1.5 mg/kg acepromazine.

The right hind limb is passed through a polycarbonate tube with a diameter of 25 mm and taped to an acrylic frame with the ankle joint at a 90° angle and the knee joint at 180°. The polycarbonate tube is affixed to a sliding platform that maintains it perpendicular to the aperture of the pQCT. The platform is adjusted so that the distal end of the femur and the proximal end of the tibia is in the scanning field. A two dimensional scout view is run for a length of 10 mm and a line resolution of 0.2 mm. After the scout view is displayed on the monitor, the proximal end of the tibia is located. The pQCT scan is initiated 3.4 mm distal from this point. The pQCT scan is 1 mm thick, has a voxel (three dimensional pixel) size of 0.140 mm, and consists of 145 projections through the slice.

After the pQCT scan is completed, the image is displayed on the monitor. A region of interest including the tibia but excluding the fibula is outlined. The soft tissue is mathematically removed using an iterative algorithm. The density of the remaining bone (total density) is reported in mg/cm³. The outer 55% of the bone is mathematically peeled away in a concentric spiral. The density of the remaining bone (Trabecular density) is reported in mg/cm³.

One week after BMD evaluation the rats are euthanized by CO₂ asphyxiation and pneumothorax, and blood is collected for cholesterol determination. The uteri also are removed and weighed after trimming associated fat and expressing any luminal fluid. Total cholesterol is determined using a Boehringer-Mannheim Hitachi 911 clinical analyzer (Roche, Alameda, Calif.) using the Cholesterol/HP kit. Statistics were compared using one-way analysis of variance with Dunnet's test.

The following results were obtained with representative metabolites of compounds of the invention (Table 5).

TABLE 5
Evaluation of Bone Mineral Density in the Ovariectomized Rat After
Administration of Select Metabolites of Compounds of the linvention

| | | Trabecular Bone |
|---|---|---|
| | Total Bone Mineral | Mineral Density |
| | Density (mean | (mean mg/cm³ ± |
| Compound | mg/cm³ ± SEM) | SEM) |
| Vehicle | 543.49 ± 14.24 | 353.96 ± 13.46 |
| 17β-estradiol (2 μg/rat) | 639.49 ± 14.47 | 453.28 ± 24.93 |
| Example 24 (10 mg/kg) | 517.56 ± 9.67 | 321.16 ± 9.04 |
| Example 21 (10 mg/kg) | 501.40 ± 11.97 | 312.34 ± 19.73 |
| Example 20 (10 mg/kg) | 525.51 ± 7.93 | 287.56 ± 17.56 |
| Example 20 (10 mg/kg) + | 682.41 ± 24.01 | 491.43 ± 36.43 |
| 17β-estradiol (2 μg/rat) | | |
| Sham operated | 685.28 ± 15.68 | 510.96 ± 16.99 |
| (no manipulation) | | |

Evaluation of Antioxidant Activity

Porcine aortas are obtained from an abattoir, washed, transported in chilled PBS, and aortic endothelial cells are harvested. To harvest the cells, the intercostal vessels of the aorta are tied off and one end of the aorta clamped. Fresh, sterile filtered, 0.2% collagenase (Sigma Type I) is placed in the vessel and the other end of the vessel then clamped to form a closed system. The aorta is incubated at 37° C. for 15-20 minutes, after which the collagenase solution is collected and centrifuged for 5 minutes at 2000×g. Each pellet is suspended in 7 mL of endothelial cell culture medium consisting of phenol red free DMEM/Ham's F12 media supplemented with charcoal stripped FBS (5%), NuSerum (5%), L-glutamine (4 mM), penicillin-streptomycin (1000 U/ml, 100 μg/ml) and gentamycin (75 μg/ml), seeded in 100 mm petri dish and incubated at 37° C. in 5% CO₂. After 20 minutes, the cells are rinsed with PBS and fresh medium added, this was repeated again at 24 hours. The cells are confluent after approximately 1 week. The endothelial cells are routinely fed twice a week and, when confluent, trypsinized and seeded at a 1:7 ratio. Cell mediated oxidation of 12.5 μg/mL LDL is allowed to proceed in the presence of the compound to be evaluated (5 μM) for 4 hours at 37° C. Results are expressed as the percent inhibition of the oxidative process as measured by the TBARS (thiobarbituric acid reactive substances) method for analysis of free aldehydes [Yagi K., Biochemical Medicine 15: 212-6 (1976)].

Progesterone Receptor mRNA Regulation Standard Pharmacological Test Procedure

This test procedure can be used to evaluate the estrogenic or antiestrogenic activity of compounds from this invention [Shughrue, et al., Endocrinology 138: 5476-5484 (1997)]. Data for representative metabolites of compounds of the invention are shown in Table 6.

TABLE 6
Effect of Representative Metabolites of Ccompounds of the Invention on
Regulation of Progesterone mRNA in the Preoptic Area of the Rat Brain

| Compound | Progesterone receptor mRNA |
|---|---|
| (10 mg/kg) | (arbitrary units; mean ± stdev) |
| Vehicle | 22.0 ± 10.1 |
| Example 21 | 110.5 ± 19.3 |
| Example 20 | 238.6 ± 36.3 |
| Example 12 | 256.2 ± 42.3 |
| Vehicle | 189.2 ± 27.2 |
| Example 34 | 511.5 ± 23.7 |
| Example 25 | 447.0 ± 60.7 |
| Example 26 | 467.8 ± 66.7 |
| Example 64 | 431.3 ± 65.6 |

Rat Hot Flush Test Procedure

The effect of test compounds on hot flushes can be evaluated in a standard pharmacological test procedure that measures the ability of a test compound to blunt the increase in tail skin temperature, which occurs as morphine-addicted rats are acutely withdrawn from the drug using naloxone [Merchenthaler, et al., Maturitas 30: 307-16 (1998)]. It can also be used to detect estrogen receptor antagonist activity by co-dosing test compound with the reference estrogen. The following data were obtained from representative metabolites of compounds of the invention (Table 7).

TABLE 7
Effect of Select Metabolites of Compounds of the Invention
in a Rat Model of Hot Flush

| | Temperature change 15 |
|---|---|
| | minutes after naloxone |
| | injection (mean ± SEM) |
| | 4.63 ± 0.79 |
| 17α-ethinyl, 17β-estradiol | 2.12 ± 1.14 |
| (0.3 mg/kg) | |
| Example 20 (15 mg/kg) | 5.28 ± 0.71 |
| Example 41 (15 mg/kg) | 5.25 ± 0.72 |

Evaluation of Vasomotor Function in Isolated Rat Aortic Rings

Sprague-Dawley rats (240-260 grams) are divided into 4 groups:
1. Normal non-ovariectomized (intact)
2. Ovariectomized (ovex) vehicle treated
3. Ovariectomized 17β-estradiol treated (1 mg/kg/day)
4. Ovariectomized animals treated with test compound (various doses)

Animals are ovariectomized approximately 3 weeks prior to treatment. Each animal receives either 17-β estradiol sulfate (1 mg/kg/day) or test compound suspended in distilled, deionized water with 1% tween-80 by gastric gavage. Vehicle treated animals received an appropriate volume of the vehicle used in the drug treated groups.

Animals are euthanized by CO₂ inhalation and exsanguination. Thoracic aortae are rapidly removed and placed in 37° C. physiological solution with the following composition (mM): NaCl (54.7), KCl (5.0), NaHCO₃ (25.0), MgCl₂ 2H₂O (2.5), D-glucose (11.8) and CaCl₂ (0.2) gassed with CO₂/O₂, 95%/5% for a final pH of 7.4. The advantitia is removed from the outer surface and the vessel is cut into 2-3 mm wide rings. The rings are suspended in a 10 mL tissue bath with one end attached to the bottom of the bath and the other to a force transducer. A resting tension of 1 gram is placed on the rings. The rings are equilibrated for 1 hour, signals are acquired and analyzed.

After equilibration, the rings are exposed to increasing concentrations of phenylephrine (10⁻⁸ to 10⁻⁴ M) and the tension recorded. The baths are then rinsed 3 times with fresh buffer. After washout, 200 mM nitro-L-arginine-methyl ester (L-NAME) is added to the tissue bath and equilibrated for 30 minutes. The phenylephrine concentration response curve is then repeated.

Evaluation of Cardioprotective Activity

Apolipoprotein E-deficient C57/B1J (apo E KO) mice were obtained from Taconic. All animal procedures were performed under strict compliance to Institutional Animal Care and Use Committee (IACUC) guidelines. Ovariectomized female apo E KO mice, 4-7 weeks of age, were housed in shoe-box cages and allowed free access to food and water. The animals were randomized by weight into groups (n=12-15 mice per group). The animals were dosed with test compounds or estrogen (17β-estradiol sulfate at 1 mg/kg/day) in the diet using a Precise-dosing Protocol, where the amount of diet consumed is measured weekly, and the dose adjusted accordingly, based on animal weight. The diet used was a Western-style diet (57U5) that is prepared by Purina® and contains 0.50% cholesterol, 20% lard and 25 IU/KG Vitamin E. The animals were dosed/fed using this paradigm for a period of 12 weeks. Control animals are fed the Western-style diet and receive no compound. At the end of the study period, the animals were euthanized and plasma samples obtained. The hearts were perfused in situ, first with saline and then with neutral buffered 10% formalin solution.

For the determination of plasma lipids and lipoproteins, total cholesterol and triglycerides are determined using enzymatic methods with commercially available kits from Boehringer Mannheim (Roche, Alameda, CA) and Wako Biochemicals (Osaka, Japan), respectively, and analyzed using the Boehringer Mannheim Hitachii 911 Analyzer. Separation and quantification of plasma lipoproteins were performed using FPLC size fractionation. Briefly, 50-100 mL of serum was filtered and injected into Superose® 12 and Superose® 6 columns connected in series and eluted at a constant flow rate with 1 mM sodium EDTA and 0.15 M NaCl. Areas of each curve representing Very Low Density Lipoprotein (VLDL), (LDL) and High Density Lipoprotein (HDL) were integrated using Waters Millennium™ software, and each lipoprotein fraction quantified by multiplying the Total Cholesterol value by the relative percent area of each respective chromatogram peak.

For the quantification of aortic atherosclerosis, the aortas were carefully isolated and placed in formalin fixative for 48-72 hours before handling. Atherosclerotic lesions were identified using Oil Red O staining. The vessels were briefly destained, and then imaged using a Nikon SMU800 microscope fitted with a Sony 3CCD video camera system in concert with IMAQ Configuration Utility (National Instrument, Austin, Tex.) as the image capturing software. The lesions were quantified en face along the aortic arch using a custom threshold utility software package (Coleman Technologies, Surrey, BC, Canada). Automated lesion assessment was performed on the vessels using the threshold function of the program, specifically on the region contained within the aortic arch from the proximal edge of the brachio-cephalic trunk to the distal edge of the left subclavian artery. Aortic atherosclerosis data were expressed as percent lesion involvement strictly within this defined luminal area.

Evaluation of Cognition Enhancement

Ovariectomized rats (n=50) are habituated to an 8-arm radial arm maze for 10-minute periods on each of 5 consecutive days. Animals are water-deprived prior to habituation and testing. A 100 μL aliquot of water placed at the ends of each arm serves as reinforcement. Acquisition of a win-shift task in the radial arm maze is accomplished by allowing the animal to have access to one baited arm. After drinking, the animal exits the arm and re-enters the central compartment, where it now has access to the previously visited arm or to a novel arm. A correct response is recorded when the animal chooses to enter a novel arm. Each animal is given 5 trials per day for 3 days. After the last acquisition trial, the animals are assigned to one of the following 4 groups:
1. Negative controls: injected with 10% DMSO/sesame oil vehicle once daily for 6 days (1 mL/kg, SC)
2. Positive controls: injected with 17β-estradiol benzoate for 2 days and tested 4 days after the second injection (17β-estradiol benzoate at 10 βg/0.1 mL per rat)
3. Estradiol: injected with 17β-estradiol will be injected daily for 6 days (20 μg/kg, SC)
4. Test compound: injected daily for 6 days (doses vary).
   All injections will. begin after testing on the last day of acquisition. The last injection for groups 1, 3, and 4 will take place 2 hours before testing for working memory.

The test for working memory is a delayed non-matching-to-sample task (DNMS) utilizing delays of 15, 30, or 60 seconds. This task is a variation of the acquisition task in which the rat is placed in the central arena and allowed to enter one arm as before. A second arm is opened once the rat traverses halfway down the first arm, and again the rat is required to choose this arm. When it has traveled halfway down this second arm, both doors are closed and the delay is instituted. Once the delay has expired, both of the original two doors, and a third novel door, are opened simultaneously. A correct response is recorded when the animal travels halfway down the third, novel arm. An incorrect response is recorded when the animal travels halfway down either the first or second arms. Each animal will receive 5 trials at each of the three delay intervals for a total of 15 trials per subject.

Evaluation of Effect on Pleurisy

The ability to reduce the symptoms of experimentally-induced pleurisy in rats can be evaluated according to the procedure of Cuzzocrea S., et al. [Endocrinology 141(4): 1455-63 (2000)].

Evaluation of Protection Against Glutamate-Induced Cytotoxicity (Neuroprotection)

The neuroprotective activity of compounds of this invention, or metabolites thereof, can be evaluated in an in vitro standard pharmacological test procedure using glutamate challenge [Zaulyanov, et al., Cellular & Molecular Neurobiology 19: 705-18 (1999); Prokai, et al., Journal of Medicinal Chemistry 44: 110-4 (2001)].

Evaluation in the Mammary End Bud Test Procedure by Histological Examination

Estrogens are required for full ductal elongation and branching of the mammary ducts, and the subsequent development of lobulo-alveolar end buds under the influence of progesterone. The non-mammotrophic activity of compounds can be determined by histological assessment of their ability to facilitate the development of lobular-alveolar end buds. Examples of such determination by histological examination are well known in the art. See, for example, Harris, H. A., et al., Endocrinology 144(10): 4241-4249 (2003); Mulac-Jericevic, B., et al., Proc. Natl. Acad. Sci. 100(17): 9744-9749 (2003); Bocchinfuso, W. P., et al., Endocrinology 141(8): 2982-2994 (2002); and Lewis, B. C., et al., Toxicological Sciences 62: 46-53 (2001), each of which is incorporated by reference herein in its entirety. In the context of the present invention, a compound is considered “non-mammotrophic” if it has activity that is <10% as efficacious as 17beta-estradiol at facilitating the development of lobular-alveolar end buds as assessed by histological examination.

Evaluation in the HLA Rat Standard Pharmacological Test Procedure for Inflammatory Bowel Disease

Representative metabolites of compounds of the invention were evaluated in the HLA rat standard pharmacological test procedure, which emulates inflammatory bowel disease in humans. The following briefly describes the procedure used and results obtained. Male HLA-B27 rats were obtained from Taconic and provided unrestricted access to food (PMI® Lab Diet® 5001, Purina Mills, Inc., St. Louis) and water. Stool quality was observed daily and graded according to the following scale: Diarrhea=3; soft stool=2; normal stool=1. At the end of the study, serum was collected and stored at −70° C. A section of colon was prepared for histological analysis and an additional segment was analyzed for myeloperoxidase activity.

In Study A, rats (22-26 weeks old) were dosed subcutaneously once per day for seven days with one of the regimens listed below. There were five rats in each group and the last dose was administered two hours before euthanasia.
Vehicle (50% DMSO/50% Dulbecco's PBS)
Example 24 (50 mg/kg)

The results from Study A are shown in Table 8. Rats dosed with vehicle continued to have diarrhea throughout the course of the study. Stool quality was improved in rats treated with Example 24.

TABLE 8
Evaluation of Stool Character of HLA Rats Treated Subcutaneously for 5
Days With Representative Compounds of the Invention

| | | Stool Character* |
|---|---|---|
| Day | Vehicle | Example 24 (50 mg/kg) |
| 1 | 3 | 2.8 |
| 2 | 3 | 2 |
| 3 | 3 | 1.8 |
| 4 | 3 | 1.6 |
| 5 | 3 | 1.6 |
| 6 | 3 | 1.4 |

*Value reported is the group's average score.
3 = diarrhea;
2 = soft stool;
1 = normal stool

In Study B, rats (8-10 weeks old) were dosed orally for twenty-six days as follows:
Vehicle (2% Tween-80/0.5% methylcellulose)
Example 25 (10 mg/kg from days 1-14; then increased to 20 mg/kg at day 15)
Example 34 (10 mg/kg)

The following results were obtained (Table 9) and show that stool character improved in all rats treated with representative metabolites of compounds of the invention.

TABLE 9
Evaluation of Stool Character of HLA Rats Treated Orally With Vehicle
or Representative Metabolites of Compounds of the Invention

Stool Character*

| Day | Vehicle | Example 25 | Example 34 |
|---|---|---|---|
| 1 | 1 | 1 | 1 |
| 2 | 1 | 1 | 1 |
| 3 | 1 | 1.25 | 1 |
| 4 | 1.25 | 1.25 | 1.25 |
| 5 | 2.5 | 1.75 | 2 |
| 6 | 2.75 | 1.5 | 1.75 |
| 7 | 2.75 | 2 | 1.75 |
| 8 | 2.75 | 2 | 1.5 |
| 9 | 3 | 1.75 | 1.5 |
| 10 | 3 | 1.5 | 1.25 |
| 11 | 2.75 | 2 | 1.5 |
| 12 | 2.75 | 1.75 | 1.5 |
| 13 | 2.75 | 2.25 | 1.25 |
| 14 | 2.75 | 2 | 1.25 |
| 15 | 2.75 | 2 | 1.25 |
| 16 | 3 | 1.5 | 1 |
| 17 | 2.75 | 1.5 | 1 |
| 18 | 2.75 | 1.5 | 1.25 |
| 19 | 2.75 | 1.25 | 1 |
| 20 | ND | ND | ND |
| 21 | ND | ND | ND |
| 22 | 3 | 1.25 | 1 |
| 23 | 3 | 1.25 | 1 |
| 24 | 3 | 1.25 | 1 |
| 25 | 3 | 1.25 | 1 |
| 26 | 3 | 1.25 | 1 |

*Value reported is the group's average score.
ND: Not determined
3 = diarrhea;
2 = soft stool;
1 = normal stool

In Study C, rats (8-10 weeks old) were dosed orally once per day for forty-six days with one of the formulations listed below. There were 4 rats in each group and the last dose was administered two hours before euthanasia.
Vehicle (2% Tween-80/0.5% methylcellulose)
Example 21 (10 mg/kg from days 1-18; then increased to 20 mg/kg at day 19)
Example 24 (10 mg/kg from days 1-24; then increased to 20 mg/kg at day 25)

The following results were obtained (Table 10) and show that stool character improved with administration of all the ERβ selective compounds.

TABLE 10
Stool Character of HLA Rats Treated Orally With Vehicle or
Representative Metabolites of Compounds of the Invention

Stool Character*

| Day | Vehicle | Example 24 | Example 21 |
|---|---|---|---|
| 1 | 2.75 | 2.75 | 2.75 |
| 2 | 3 | 2.75 | 3 |
| 3 | 3 | 2.75 | 2.75 |
| 4 | 3 | 2.5 | 2.75 |
| 5 | 3 | 2 | 2.75 |
| 6 | 3 | 2.5 | 2.5 |
| 7 | 3 | 2.25 | 2.5 |
| 8 | 3 | 2.25 | 2.75 |
| 9 | 3 | 2.25 | 2.5 |
| 10 | 3 | 2.25 | 2.75 |
| 11 | 3 | 2.25 | 2.5 |
| 12 | 3 | 1.75 | 2.5 |
| 13 | 3 | 2.25 | 2.5 |
| 14 | 3 | 2 | 2.5 |
| 15 | 3 | 1.75 | 2.5 |
| 16 | 3 | 1.75 | 2.5 |
| 17 | 3 | 1.75 | 2.5 |
| 18 | 3 | 1.75 | 2.5 |
| 19 | 3 | 1.75 | 2.75 |
| 20 | 3 | 1.75 | 2.5 |
| 21 | 3 | 1.75 | 2.75 |
| 22 | 3 | 1.75 | 2.5 |
| 23 | 3 | 1.75 | 2.25 |
| 24 | 3 | 2 | 1.75 |
| 25 | 3 | 2 | 2 |
| 26 | 2.75 | 2.25 | 2 |
| 27 | 3 | 1.75 | 2 |
| 28 | 3 | 1.75 | 2 |
| 29 | 3 | 1.5 | 2 |
| 30 | 2.75 | 1.5 | 2.25 |
| 31 | 3 | 1.5 | 2.25 |
| 32 | 3 | 1.5 | 2 |
| 33 | 3 | 1.75 | 1.5 |
| 34 | 3 | 1.75 | 1.75 |
| 35 | 3 | 1.5 | 1.5 |
| 36 | 3 | 1.5 | 1.75 |
| 37 | 3 | 1.25 | 1.5 |
| 38 | 3 | 1.75 | 1.5 |
| 39 | 3 | 1.75 | 2 |
| 40 | 3 | 1.5 | 1.75 |
| 41 | 3 | 1.75 | 2 |
| 42 | 3 | 1.5 | 2 |
| 43 | 3 | 1.5 | 2 |
| 44 | 3 | 1.5 | 2 |
| 45 | 3 | 1.25 | 2 |
| 46 | 3 | 1.25 | 2 |

3 = diarrhea; 2 = soft stool; 1 = normal stool
*Value reported is the group's average score.
3 = diarrhea;
2 = soft stool;
1 = normal stool

Histological analysis. Colonic tissue was immersed in 10% neutral buffered formalin. Each specimen of colon was separated into four samples for evaluation. The formalin-fixed tissues were processed in a Tissue-Tek® vaccum infiltration processor (Miles, Inc; West Haven, Conn.) for paraffin embedding. The samples were sectioned at 5 μm and then stained with hematoxylin and eosin (H&E) for blinded histological evaluations using a scale modified after Boughton-Smith. After the scores were completed, the samples were unblinded, and data were tabulated and analyzed by ANOVA linear modeling with multiple mean comparisons. Sections of colonic tissue were evaluated for several disease indicators and given relative scores. As shown in Table (11) (a composite of two subcutaneous dosing studies, including Study A), Example 24 is effective in reducing several measurements of tissue injury.

TABLE 11
Histological Scoring of Disease Severity in the HLA-B27 Rat Model:
Composite of Two Studies Using Subcutaneous Dosing for 5 Days

| | | | Lesion | | |
|---|---|---|---|---|---|
| | Ulceration | Inflammation | depth | Fibrosis | Total |
| Group | (0-2) | (0-3) | (0-2) | (0-2) | score |
| 2. Vehicle | 1.38 | 2.69 | 1.19 | 0.88 | 6.13 |
| Example 24 | 0.25*# | 1.05*# | 0.2# | 0* | 1.5*# |
| (50 mg/kg) | | | | | |
| Example 24 | 0.81* | 1.63* | 0.69* | 0.50* | 3.6* |
| (10 mg/kg)^{a} | | | | | |
| Example 24 | 1.25 | 1.63* | 0.88* | 0.75 | 4.4* |
| (1 mg/kg)^{a} | | | | | |

^{a}data taken from a second study
*sig < vehicle or EE + ICI
#sig < EE

Intestinal tissue from Study B (see above) was also examined histologically. As shown below (Table 12), both compounds significantly reduced total disease score.

TABLE 12
Histological Scoring of Disease Severity in the Colon
from Animals Treated Orally for 4 weeks with Representative Metabolites
of Compounds from the Invention

| | Ulceration | Inflammation | Lesion depth | Fibrosis | |
|---|---|---|---|---|---|
| Group | (0-2)** | (0-3) | (0-2) | (0-2) | Total score |
| 3. Vehicle | 1.44 ± 0.66 | 2.88 ± 0.14 | 1.56 ± 0.63 | 1.06 ± 0.32 | 6.94 ± 1.51 |
| Example | 0.44 ± 0.24* | 1.50 ± 0.35* | 0.44 ± 0.24* | 0.31 ± 0.13* | 2.69 ± 0.52* |
| 25 | | | | | |
| Example | 0.75 ± 0.46* | 1.81 ± 0.13* | 0.63 ± 0.32* | 0.31 ± 0.32* | 3.50 ± 1.10* |
| 34 | | | | | |

*sig < vehicle;
**values reported as Means ± SD

Intestinal tissue from Study C (see above) was also examined histologically. As shown below (Table 13), Example 24 significantly reduced total disease score. The scores of Example 21 on all disease parameters, although not statistically significant, were lower than corresponding scores from vehicle-treated rats.

TABLE 13
Histological Scoring of Disease Severity in the Colon from Animals
Treated Orally for 7 Weeks with Representative Metabolites of Compounds from the
Invention.

| | Ulceration | Inflammation | Lesion depth | Fibrosis | |
|---|---|---|---|---|---|
| Group | (0-2)** | (0-3) | (0-2) | (0-2) | Total score |
| 4. Vehicle | 1.19 ± 0.69 | 2.38 ± 0.32 | 1.0 ± 0.54 | 0.94 ± 0.75 | 5.50 ± 2.1 |
| Example | 0.81 ± 0.47 | 2.06 ± 0.43 | 0.75 ± 0.50 | 0.56 ± 0.32 | 4.19 ± 1.74 |
| 21 | | | | | |
| Example | 0* | 0.69 ± 0.24* | 0* | 0* | 0.69 ± 0.24* |
| 24 | | | | | |

*sig < vehicle;
**values reported as Means ± SD

Evaluation in Two Models of Arthritis

Lewis rat assay of adjuvant-induced arthritis. Sixty, female, 12 weeks old, Lewis rats are housed according to standard facility operating procedures. They receive a standard regimen of food and water ad libitum. Each animal is identified by a cage card indicating the project group and animal number. Each rat number is marked by indelible ink marker on the tail. At least 10-21 days before study, they are anesthetized and ovariectomized by standard aseptic surgical techniques.

Freund's Adjuvant-Complete (Sigma Immuno Chemicals, St. Louis, Mo.) is used to induce arthritis, each mL containing 1 mg Mycobacterium tuberculosis heat killed and dried, 0.85 mL mineral oil and 0.15 mL mannide monooleate (Lot No. 084H8800).

The following are examples of two test procedures.

Inhibition test procedure: Thirty rats are injected intradermally with 0.1 mL of Freund's Adjuvant-Complete at the base of the tail. The animals are randomized to four groups, each group containing six rats. Each day, the groups receive vehicle (50% DMSO (JT Baker, Phillipsburg, N.J.)/1× Dulbecco's phosphate saline (GibcoBRL, Grand Island, N.Y.)) or test compound (administered subcutaneously). All rats began treatment on Day 1. Data for representative metbolites of compounds of the invention are shown in Table 14.

Treatment test procedure: Thirty rats are injected intradermally with 0.1 mL of Freund's Adjuvant-Complete at the base of the tail. The animals are randomized to four groups, each group containing six rats. Each day, the groups receive vehicle (50% DMSO (JT Baker, Phillipsburg, N.J.)/1× Dulbecco's phosphate saline (GibcoBRL, Grand Island, N.Y.)) or test compound (administered subcutaneously).

All rats began treatment on Day 8 after adjuvant injection. Data for representative metabolites of compounds of the invention are shown in Tables 15, 16 and 17, hereinbelow.

Statistical analysis was performed using Abacus Concepts Super ANOVA. (Abacus Concepts, Inc., Berkeley, Calif.). All of the parameters of interest were subjected to Analysis of Variance with Duncan's new multiple range post hoc testing between groups. Data are expressed throughout as mean ± standard deviation (SD), and differences were deemed significant if p<0.05.

The degree of arthritis severity is monitored daily in terms of the following disease indices: Hindpaw erythema, hindpaw swelling, tenderness of the joints, and movements and posture. An integer scale of 0 to 3 is used to quantify the level of erythema (0=normal paw, 1=mild erythema, 2=moderate erythema, 3=severe erythema) and swelling (0=normal paw, 1=mild swelling, 2=moderate swelling, 3=severe swelling of the hind paw). The maximal score per day is 12.

At the end of the study, the rats are euthanized with CO₂, hindlimbs removed at necropsy and fixed in 10% buffered formalin, and the tarsal joints decalcified and embedded in paraffin. Histologic sections are stained with Hematoxylin and Eosin or Saffranin O-Fast Green stain.

Slides are coded so that the examiner is blinded to the treatment groups. Synovial tissue from tarsal joints is evaluated based on synovial hyperplasia, inflammatory cell infiltration, and pannus formation [Poole and Coombs,

International Archives of Allergy & Applied Immunology 54: 97-113 (1977)], as outlined below.

| Category | Grade |
|---|---|
| 1. Synovial lining cells | |
| a. No change | 0 |
| b. Cells enlarged, slightly thickened | 1 |
| c. Cells enlarged, increase in numbers, moderately thickened. | 2 |
| No villus present | |
| d. Cells enlarged, thickened. Villlus present | 3 |
| 2. Fibroplasia | |
| a. No change | 0 |
| b. Fibroplasia present under lining cells | 1 |
| c. Small areas of areolar tissue replaced by fibrous tissue | 2 |
| d. Replacement of areolar tissue by fibrous tissue | 3 |
| 3. Inflammatory cells | |
| a. Occasionally seen, scattered throughout selection | 0 |
| b. Cells present in small numbers in or just under lining cell | 1 |
| layer and/or around blood vessels. | |
| c. Small focal collection of cells may be present | 2 |
| d. Large numbers of cells present in capsule and in or under | 3 |
| lining cell layers. Large foci often seen. | |
| 4. Pannus | |
| a. Not detectable | 0 |
| b. Detectable | 1 |

In addition, articular cartilage and bone are evaluated using Mankin's histological grading system [Mankin, et al., Journal of Bone & Joint Surgery—American 53: 523-37 (1971)] as shown below.

| (i) | Grade |
|---|---|
| 1. Structure | |
| a. Normal | 0 |
| b. Surface irregularity | 1 |
| c. Pannus and surface irregularity | 2 |
| d. Clefts to transitional zone | 3 |
| e. Clefts to radial zone | 4 |
| f. Clefts to calcified zone | 5 |
| g. Complete disorganization | 6 |
| 2. Cells | |
| a. Normal | 0 |
| b. Diffuse hypercellularity | 1 |
| c. Cloning | 2 |
| d. Hypocellularity | 3 |
| 3. Safranin-O staining | |
| a. Normal | 0 |
| b. Slight reduction | 1 |
| c. Modest reduction | 2 |
| d. Severe reduction | 3 |
| e. No dye noted | 4 |
| 4. Tidemark integrity | |
| a. Intact | 0 |
| b. Crossed by blood vessels | 1 |

TABLE 14
Evaluation of Joint Inflammation of Lewis Rats: Inhibition Protocol

| Day | Vehicle | Example 24 |
|---|---|---|
| 1 | 0.00 | 0.00 |
| 2 | 0.00 | 1.00 |
| 3 | 4.50 | 4.50 |
| 4 | 5.50 | 4.83 |
| 5 | 9.33 | 5.83 |
| 6 | 10.50 | 6.16 |
| 7 | 10.60 | 6.16 |
| 8 | 11.00 | 5.33 |
| 9 | 11.50 | 5.66 |
| 10 | 11.33 | 4.33 |
| 11 | 10.83 | 3.16 |
| 12 | 10.83 | 3.16 |
| 13 | 11.00 | 2.16 |
| 14 | 11.00 | 3.33 |
| 15 | 11.00 | 3.00 |
| 16 | 11.00 | 1.66 |
| 17 | 10.50 | 1.50 |

TABLE 15
Evaluation of Joint Inflammation of Lewis Rats: Treatment Protocol

| Day | Vehicle | Example 24 | Example 27 | Example 32 |
|---|---|---|---|---|
| 1 | 10.83 | 11.33 | 11.33 | 11.33 |
| 2 | 11.00 | 11.15 | 11.15 | 10.83 |
| 3 | 10.83 | 11.33 | 11.33 | 9.33 |
| 4 | 11.33 | 9.50 | 9.83 | 8.00 |
| 5 | 11.50 | 8.00 | 8.83 | 5.83 |
| 6 | 11.50 | 7.00 | 7.83 | 3.33 |
| 7 | 11.50 | 5.83 | 6.16 | 3.00 |
| 8 | 11.50 | 4.83 | 5.00 | 2.50 |
| 9 | 11.00 | 3.50 | 4.33 | 2.50 |
| 10 | 11.00 | 3.83 | 2.66 | 2.50 |
| 11 | 10.66 | 3.83 | 1.83 | 2.50 |
| 12 | 10.66 | 3.83 | 1.83 | 2.50 |
| 13 | 10.50 | 3.16 | 2.66 | 2.50 |
| 14 | 9.83 | 3.16 | 2.66 | 2.50 |
| 15 | 8.10 | 2.83 | 2.00 | 2.00 |
| 16 | 7.35 | 2.83 | 2.00 | 1.33 |
| 17 | 6.50 | 2.00 | 1.50 | 1.00 |

TABLE 16
Histological Scoring of Synovitis in the Tarsal Joints of Lewis Rats:
Treatment Protocol

| | | | | | Total |
|---|---|---|---|---|---|
| | Synovial | | Inflammatory | | Synovitis |
| | Structure | Fibroplasia | Cells | Pannus | Score |
| Group | (0-3)** | (0-3) | (0-3) | (0-1) | (0-10) |
| 5. Vehicle | 2.58 ± 0.38 | 1.75 ± 0.42 | 2.92 ± 0.20 | 1.00 ± 0.89 | 8.25 ± 1.57 |
| Example 24 | 1.42 ± 0.49* | 0.42 ± 0.80* | 1.33 ± 0.41* | 0.08 ± 0.20* | 3.25 ± 1.54* |
| 50 mg/kg | | | | | |

*sig < vehicle;
**values reported as Mean ± SD

TABLE 17
Histological Scoring of Cartilage Change (Mankin Scores) in the Tarsal
Joints of Lewis Rats: Treatment Protocol

| | | | Saffranin-O/ | | Total |
|---|---|---|---|---|---|
| | Cartilage | Cartilage | Fast Green | Tidemark | Mankin |
| | Structure | Cells | Staining | Integrity | Score |
| Group | (0-6)** | (0-3) | (0-4) | (0-1) | (0-14) |
| 6. Vehicle | 2.83 ± 0.26 | 2.58 ± 0.38 | 2.50 ± 0.32 | 0 | 7.92 ± 0.74 |
| Example 24 | 1.58 ± 0.49* | 0.83 ± 0.75* | 1.25 ± 0.69* | 0 | 3.67 ± 1.86* |
| 50 mg/kg | | | | | |

*sig < vehicle;
**values reported as Mean ± SD

Evaluation in the HLA-B27 Rat Model of Arthritis

Representative metabolites of compounds of the invention were evaluated in the HLA-B27 rat standard pharmacological test procedure, which emulates arthritis in humans. The following briefly describes the procedure used and results obtained. Male HLA-B27 rats were obtained from Taconic and provided unrestricted access to a food (PMI® LabDiet 5001) and water. Joint scores and histology were evaluated as described above for the Lewis rat model of adjuvant-induced arthritis.

Study 1: Rats (8-10 weeks old) were dosed orally once per day for forty-six days with one of the formulations listed below. There were 4 rats in each group and the last dose was administered two hours before euthanasia.
Vehicle (2% Tween-80/0.5% methylcellulose)
Example 21 (10 mg/kg from days 1-18; then increased to 20 mg/kg at day 19)
Examples 24 (10 mg/kg from days 1-24; then increased to 20 mg/kg at day 25)

The following results were obtained for representative metabolites of compounds of the invention (Tables 18 and 19).

TABLE 18
Evaluation of Joint Inflammation from Study 1

| Day | Vehicle | Example 24 | Example 21 |
|---|---|---|---|
| 29 | 2.5 | 1.5 | 0.75 |
| 30 | 6 | 0.5 | 1.75 |
| 31 | 5 | 0.5 | 1.25 |
| 32 | 6.75 | 1.25 | 0.75 |
| 33 | 8 | 2 | 1 |
| 34 | 8 | 2.25 | 1.25 |
| 35 | 8 | 2 | 2.25 |
| 36 | 6 | 2.25 | 1 |
| 37 | 7.5 | 2 | 4 |
| 38 | 6.5 | 2.75 | 1.5 |
| 39 | 7.5 | 2.25 | 1.5 |
| 40 | 7.5 | 1.75 | 2.25 |
| 41 | 6.5 | 2 | 2.25 |
| 42 | 6.5 | 2.5 | 1.5 |
| 43 | 6 | 4.75 | 1.25 |
| 44 | 6.75 | 3 | 1 |
| 45 | 5.5 | 2.75 | 2.5 |
| 46 | 6 | 3.25 | 2 |

TABLE 19
Evaluation of Joint Histology from Study 1.

| Compound | Synovitis score (mean ± SD) | Mankin score (mean ± SD) |
|---|---|---|
| Vehicle | 7.75 ± 2.6 | 6.75 ± 1.0 |
| Example 24 | 3.17 ± 0.3* | 3.5 ± 1.8** |
| Example 21 | 6.1 ± 0.75 | 4.6 ± 0.9 |

*sig < vehicle, p < 0.07
**sig < vehicle, p < 0.05

Study 2: Rats (8-10 weeks old) were dosed orally for twenty-six days with one of the formulations listed below. There were 4 rats in each group and the last dose was administered two hours before euthanasia.
Vehicle (2% Tween-80/0.5% methylcellulose)
Example 25 (10 mg/kg from days 1-14; then increased to 20 mg/kg at day 15)
Example 34 (10 mg/kg)

The following results were obtained for representative metabolites of compounds of the invention (Table 20).

TABLE 20
Evaluation of Joint Inflammation of HLA Rats from Study 2.

| Day | Vehicle | Example 25 | Example 34 |
|---|---|---|---|
| 1 | 0 | 0 | 0 |
| 2 | 0 | 0 | 0 |
| 3 | 0 | 0 | 0 |
| 4 | 0 | 0 | 0 |
| 5 | 0 | 0 | 0 |
| 6 | 0 | 0 | 0 |
| 7 | 0 | 0 | 0 |
| 8 | 2.5 | 1 | 0.25 |
| 9 | 3.75 | 2 | 0.75 |
| 10 | 2.75 | 2.25 | 0.5 |
| 11 | 3.5 | 2.25 | 0.5 |
| 12 | 1.25 | 2 | 0.25 |
| 13 | 1.25 | 2 | 0.5 |
| 14 | 1.25 | 2 | 0 |
| 15 | 5.25 | 3.75 | 0.5 |
| 16 | 4.5 | 3 | 0.5 |
| 17 | 3.5 | 2.75 | 0.25 |
| 18 | 3.75 | 2 | 0.75 |
| 19 | 5.5 | 1.5 | 1 |
| 22 | 3.25 | 1.25 | 1 |
| 23 | 6.5 | 2.5 | 1.75 |
| 24 | 6.5 | 2 | 1.75 |
| 25 | 6.25 | 2 | 2 |
| 26 | 7 | 1.75 | 3 |

Evaluation in in vivo Models of Carcinogeneisis

The ability of compounds of this invention, and metabolites thereof, to treat and inhibit various malignancies or hyperprolific disorders can be evaluated in standard pharmacological test procedures that are readily available in the literature, and include the following two procedures.

Breast cancer. Athymic nu/nu (nude) mice are obtained ovariectomized from Charlse River Laboratories (Wilmington, Mass.). One day prior to tumor cell injection, animals are implanted with time-release pellets containing 0.36-1.7 mg 17β-estradiol (60 or 90 day release, Innovative Research of America, Sarasota, Fla.) or a placebo. The pellet is introduced subcutaneously into the intrascapular region using a 10-gauge precision trochar. Subsequently, mice are injected subcutaneously into the breast tissue with either 1×10⁷ MCF-7 cells or 1×10⁷ BG-1 cells. The cells are mixed with an equal volume of matrigel, a basement membrane matrix preparation to enhance tumor establishment. Test compounds can be evaluated either by dosing one day after tumor cell implantation (inhibition regimen) or after tumors have reached a certain size (treatment regimen). Compounds are administered either intraperitoneally or orally in a vehicle of 1% Tween-80 in saline each day. Tumor size is evaluated every three or seven days.

Colon cancer. The ability to treat or inhibit colon cancer can be evaluated in the test procedure of Smirnoff P., et al. [Oncology Research 11: 255-64 (1999)].

Evaluation of Neuroprotection in Two in vivo Test Procedures

Transient global ischemia in the Mongolian gerbil. The effect of test compounds on preventing or treating brain injury in response to oxygen deprivation/reperfusion were measured using the following test procedure.

Female Mongolian gerbils (60-80 g; Charles River Laboratories, Kingston, N.Y.) were housed in the Wyeth-Ayerst animal care facility Association for Assessment and Acreditation of Laboratory Animal Care (AAALAC) certified with a 12-hour light, 12-hour dark photoperiod and free access to tap water and a low-estrogen casein diet (Purina®; Richmond, Ind.). After acclimation (3-5 days), gerbils were anesthetized with isoflurane (2-3% mixture with O₂), ovariectomized (Day 0). Beginning the following morning (Day 1), gerbils were treated subcutaneously each day with either vehicle (10% ETOH/corn oil), 17β-estradiol (1 mg/kg, sc) or an experimental compound. On Day 6, gerbils (n=4-5/group) were anesthetized with isoflurane, the common carotid arteries visualized via a mid-line neck incision and both arteries simultaneously occluded for 5 minutes with non-traumatic micro aneurysm clips. After occlusion, the clips were removed to allow cerebral reperfusion and the neck incision closed with wound clips. All animals were fasted overnight prior to the global ischemia surgery, a step that facilitates consistent ischemic injury. On Day 12, gerbils were exposed to a lethal dose of CO₂, and the brains frozen on dry ice and stored at −80° C. The animal protocols used for these studies were reviewed and approved by the Radnor/Collegeville Animal Care and Use Committee (RACUC/CACUC) at Wyeth-Ayerst Research.

The degree of neuronal protection was evaluated by in situ hybridization analysis of neurogranin mRNA. Briefly, 20 μm coronal cryostat sections were collected on gelatin-coated slides, dried and stored at −80° C. At the time of processing, the desiccated slide boxes were warmed to room temperature, the slides posffixed in 4% paraformaldehyde, treated with acetic anhydride and then delipidated and dehydrated with chloroform and ethanol. Processed section-mounted slides were then hybridized with 200 μl (6×10⁶ DPM/slide) of an antisense or sense (control) riboprobe for Neurogranin (³⁵S-UTP-labeled NG-241; bases 99-340) in a 50% formamide hybridization mix and incubated overnight at 55° C. in a humidified slide chamber without coverslipping. The following morning, the slides were collected in racks, immersed in 2×SSC (0.3 M NaCl, 0.03 M sodium citrate; pH 7.0)/10 mM DTT, treated with RNase A (20 μg/ml) and washed (2×30 min) at 67° C. in 0.1×SSC to remove nonspecific label. After dehydration, the slides were opposed to BioMax® (BMR-1; Kodak, Rochester, N.Y.) X-ray film overnight.

The level of neurogranin hybridization signal was used to quantitatively assess the degree of neuronal loss in the CA1 region after injury and to evaluate the efficacy of 17β-estradiol and experimental compounds. Neurogranin mRNA was selected for these studies because it is highly expressed in the hippocampal neurons including CA1, but absent in glia and other cell types present in this brain region. Therefore, measurement of the amount of neurogranin mRNA present represents surviving neurons. Relative optical density measurements of neurogranin hybridization signal were obtained from film autoradiograms with a computer based image analysis system (C-Imaging Inc., Pittsburgh, Pa.). The results from 6 sections (40 μm apart) per animal were averaged and statistically evaluated. Numerical values are reported as the mean ± SEM. One-way analysis of variance was used to test for differences in the level of neurogranin mRNA and all statements of non-difference in the results section imply that p>0.05.

The following results were obtained with representative metabolites of compounds of the invention (Table 21).

TABLE 21
Effect of Representative Metabolites of Compounds of the Invention on
Preserving Neurons in the Gerbil Hippocampus

| | Neurogranin mRNA |
|---|---|
| | (arbitrary units, mean ± stdev) |
| Vehicle | 0.0 |
| Example 24 | 0.0 |
| Example 41 | 43.0 ± 21.8 |

Middle cerebral artery occlusion in mice. Neuroprotection can be evaluated according to the test procedures described by Dubal [see, Dubal, et al., Proceedings of the National Academy of Sciences of the United States of America 98: 1952-1957 (2001) and Dubal, et al., Journal of Neuroscience 19: 6385-6393 (1999)].

Ovulation Inhibition Standard Pharmacological Test Procedure

The test procedure is used to determine whether test compounds can inhibit or change the timing of ovulation. It can also be used to determine the number of oocytes ovulated [Lundeen, et al., J Steroid Biochem Mol Biol 78: 137-143 (2001)]. The following data were obtained from representative metabolites of compounds from the invention (Table 22).

TABLE 22
Effect of representative metabolites of compounds from the invention on
inhibiting ovulation.

| Compound | Number of oocytes (mean ± SEM) |
|---|---|
| Vehicle | 13.00 ± 0.72 |
| Example 20 (50 mg/kg) | 14.13 ± 0.79 |
| Example 24 (50 mg/kg) | 13.86 ± 0.77 |

Evaluation in an Endometriosis Standard Pharmacologic Test Procedure

This procedure is slightly modified from a published method [Bruner-Tran. et al., Journal of Clinical Investigation 99: 2851-2857 (1997)]. In brief, normal human endometrial tissue (cycle day ˜12) is treated in vitro overnight with 10 nM 17β-estradiol and then implanted into ovariectomized athymic nude mice. For the purposes of these studies, the mice do not receive estrogen/placebo implants, as described in the paper. Lesions are allowed to establish for at least 10 days, then oral daily dosing begins and continues for at least 15 days. It should be noted that all mice have visible lesions at the start of dosing. At necropsy, the number of mice with lesions is determined, as well as the lesions per mouse.

The compound of Example 24 was evaluated three times in this procedure at a dose of 10 mg/kg. In each test procedure, mice dosed with the compound of Example 24 had fewer lesions at necropsy than those mice dosed with vehicle. For example, in Study 1, each of the four mice in the vehicle group had at least one lesion and there were 10 total lesions in this group. In contrast, only two of six mice treated with Example 24 had any lesions and only one lesion was found per animal. Therefore, because all mice had lesions at the start of treatment, the compound of Example 24 caused lesion regression in four of six mice

Based on the results obtained in the standard pharmacological test procedures, the prodrug compounds of this invention are expected to yield compounds that are estrogen receptor modulators useful in the treatment or inhibition of conditions, disorders, or disease states that are at least partially mediated by an estrogen deficiency or excess, or which may be treated or inhibited through the use of an estrogenic agent. Such compounds are particularly useful in treating a peri-menopausal, menopausal, or postmenopausal patient in which the levels of endogenous estrogens produced are greatly diminished. Menopause is generally defined as the last natural menstrual period and is characterized by the cessation of ovarian function, leading to the substantial diminution of circulating estrogen in the bloodstream. As used herein, menopause also includes conditions of decreased estrogen production that may be caused surgically, chemically, or by a disease state that leads to premature diminution or cessation of ovarian function.

The prodrug compounds of the invention are also useful in inhibiting or treating other effects of estrogen deprivation including, hot flushes, vaginal or vulvar atrophy, atrophic vaginitis, vaginal dryness, pruritus, dyspareunia, dysuria, frequent urination, urinary incontinence, urinary tract infections. Other reproductive tract uses include the treatment or inhibition of dysfunctional uterine bleeding. The compounds are also useful in treating or inhibiting endometriosis.

The prodrug compounds of this invention are also active in the brain and therefore, are useful for inhibiting or treating Alzheimer's disease, cognitive decline, decreased libido, senile dementia, neurodegenerative disorders, depression, anxiety, insomnia, schizophrenia, and infertility. The compounds of this invention are also useful in treating or inhibiting benign or malignant abnormal tissue growth including, glomerulosclerosis, prostatic hypertrophy, uterine leiomyomas, breast cancer, scleroderma, fibromatosis, endometrial cancer, polycystic ovary syndrome, endometrial polyps, benign breast disease, adenomyosis, ovarian cancer, melanoma, prostate cancer, cancers of the colon, CNS cancers, such as glioma or astioblastomia.

The prodrug compounds of this invention are cardioprotective and are antioxidants, and are useful in lowering cholesterol, triglycerides, Lp(a), and LDL levels; inhibiting or treating hypercholesteremia, hyperlipidemia, cardiovascular disease, atherosclerosis, peripheral vascular disease, restenosis, and vasospasm, and inhibiting vascular wall damage from cellular events leading toward immune mediated vascular damage.

The prodrug compounds of this invention are also useful in treating disorders associated with inflammation or autoimmune diseases, including inflammatory bowel disease (Crohn's disease, ulcerative colitis, indeterminate colitis), arthritis (rheumatoid arthritis, spondyloarthropathies, osteoarthritis), pleurisy, ischemia/reperfusion injury (e.g., stroke, transplant rejection, myocardial infarction, etc.), asthma, giant cell arteritis, prostatitis, uveitis, psoriasis, multiple sclerosis, systemic lupus erythematosus and sepsis.

The prodrug compounds of this invention are also useful in treating or inhibiting ocular disorders including cataracts, uveitis, and macular degeneration and in treating skin conditions such as aging, alopecia, and acne.

The prodrug compounds of this invention are also useful in treating or inhibiting metabolic disorders such as type-II diabetes, of lipid metabolism, appetite (e.g., anorexia nervosa and bulimia).

Prodrug compounds in this invention are also useful in treating or inhibiting bleeding disorders such as hereditary hemorrhagic telangiectasia, dysfunctional uterine bleeding, and combating hemorrhagic shock.

Prodrug compounds of this invention are useful in disease states where amenorrhea is advantageous, such as leukemia, endometrial ablations, chronic renal or hepatic disease or coagulation diseases or disorders.

The prodrug compounds of this invention can be used as a contraceptive agent, particularly when combined with a progestin.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds of this invention may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. Useful tablet formulations may be made by conventional compression and wet granulation or dry granulation methods, and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The prodrug compounds of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

### EXAMPLES

The preparation of representative examples of compounds that can be derivatized to form compounds of the invention is described below.

### Example 1

### 2-(5-HYDROXY-1,3-BENZOXAZOL-2-YL) BENZENE-1,4-DIOL

### Step a) n-(2,5-dimethoxyphenyl)-2,5-dimethoxybenzamide

A mixture of 2,5-dimethoxybenzoic acid (5.0 g, 27.5 mmol) and thionyl chloride (15 mL) was refluxed for 1 hour. The volatiles then were removed under vacuum. The residue was dissolved in THF (20 mL) and added into a cold (0° C.) solution of 2,5-dimethoxyaniline (4.6 g, 30.2 mmol), triethylamine (5 mL, 35.9 mmol) and THF (40 mL). The reaction mixture was stirred for 30 mins., poured into water, acidified with HCl (2N) and extracted with EtOAc. The organic extracts were dried over MgSO₄. Evaporation and purification by flash chromatography (hexanes/EtOAc 2/1) gave a white solid (8.1 g, 93% yield, m.p. 121-123° C.); MS m/e 318 (M+H)⁺.

Analysis for: C₁₇H₁₉NO₅ Calc'd: C, 64.34; H, 6.03; N, 4.41 Found: C, 64.29; H, 5.95; N, 4.44

### Step b) 2-(5-Hydroxy-1,3-benzoxazol-2-yl) benzene-1,4-diol

A mixture of N-(2,5-dimethoxyphenyl)-2,5-dimethoxybenzamide (1.0 g, 3.1 mmol) and pyridine hydrochloride (2.0 g, 17.3 mmol) was stirred at 200° C. for 1 hour. The reaction mixture was cooled to room temperature and HCl (10 mL, 2 N) was added. The reaction mixture was then extracted with EtOAc and the organic extracts were dried over MgSO₄. Evaporation and purification by flash chromatography (hexanes/EtOAc 2/1) gave a white solid (0.8 g, 76% yield, m.p. 309-311° C.); MS m/e 242 (M−H)⁺.

Analysis for: C₁₃H₉NO₄ Calc'd: C, 64.20; H, 3.73; N, 5.76 Found: C, 63.98; H, 3.71; N, 5.62

### Example 2

### 3-(5-HYDROXY-1,3-BENZOXAZOL-2-YL)BENZENE-1,2-DIOL

The title compound was prepared in substantially the same manner as described in Example 1, from 2,5-dimethoxyaniline and 2,3-dimethoxybenzoic acid. The product was obtained as a tan solid, m.p. 239-241° C.; MS m/e 244 (M+H)⁺.

Analysis for: C₁₃H₉NO₄ Calc'd: C, 64.20; H, 3.73; N, 5.76 Found: C, 63.86; H, 3.90; N, 5.74

### Example 3

### 2-(3-FLUORO-4-HYDROXYPHENYL)-1,3-BENZOXAZOL-5-OL

The title compound was prepared in substantially the same manner as described in Example 1, from 2,5-dimethoxyaniline and 3-fluoro-4-methoxybenzoic acid, and was obtained as a white solid, m.p. 262-268° C.; MS m/e 244 (M−H)⁺.

Analysis for: C₁₃H₈FNO₃ Calc'd: C, 63.68; H, 3.29; N, 5.71 Found: C, 64.01; H, 3.25; N, 5.63

### Example 4

2-(3-CHLORO-4-HYDROXYPHENYL)-1,3-BENZOXAZOL-5-OL

The title compound was prepared in substantially the same manner as described in Example 1, from 2,5-dimethoxyaniline and 3-chloro-4-methoxybenzoic acid and was obtained as a white solid, m.p. 254-256° C.; MS m/e 260 (M−H)⁺.

Analysis for: C₁₃H₈CINO₃ Calc'd: C, 59.67; H, 3.08; N, 5.35 Found: C, 59.59; H, 3.02; N, 5.25

### Example 5

### 2-(2-CHLORO-4-HYDROXYPHENYL)-1,3-BENZOXAZOL-5-OL

The title compound was prepared in substantially the same manner as described in Example 1, from 2,5-dimethoxyaniline and 2-chloro-4-methoxybenzoic acid, and was obtained as a white solid, m.p. 253-255° C.; MS m/e 262 (M+H)⁺.

Analysis for: C₁₃H₈CINO₃ Calc'd: C, 59.67; H, 3.08; N, 5.35 Found: C, 59.79; H, 2.87; N, 5.36

### Example 6

### 2-(3-FLUORO-4-HYDROXYPHENYL)-1,3-BENZOXAZOL-6-OL

The title compound was prepared in substantially the same manner as described in Example 1, from 2,4-dimethoxyaniline and 3-fluoro-4-methoxybenzoic acid, and was obtained as a white solid, m.p. 269-271° C.; MS m/e 244 (M−H)⁺.

Analysis for: C₁₇H₁₇NO₃ Calc'd: C, 63.68; H, 3.29; N, 5.71 Found: C, 63.53; H, 3.71; N, 5.38

### Example 7

### 2-(3-TERT-BUTYL-4-HYDROXYPHENYL)-1,3-BENZOXAZOL-6-OL

The title compound was prepared in substantially the same manner as described in Example 1, from 2,4-dimethoxyaniline and 3-tert-butyl-4-methoxybenzoic acid, and was obtained as a white solid, m.p. 220-222° C.; MS m/e 284 (M+H)⁺.

Analysis for: C₁₇H₁₇NO₃ Calc'd: C, 72.07; H, 6.05; N, 4.94 Found: C, 72.03; H, 6.43; N, 4.72

### Example 8

### 2-(6-HYDROXY-1,3-BENZOXAZOL-2-YL)BENZENE-1,4-DIOL

The title compound was prepared in substantially the same manner as described in Example 1, from 2,4-dimethoxyaniline and 2,5-dimethoxybenzoic acid, and was obtained as a tan solid, m.p. 278-280° C.; MS m/e 244 (M+H)⁺.

Analysis for: C₁₃H₉NO₄ Calc'd: C, 64.20; H, 3.73; N, 5.76 Found: C, 64.09; H, 3.14; N, 5.65

### Example 9

### 3-(6-HYDROXY-1,3-BENZOXAZOL-2-YL)BENZENE-1,2-DIOL

The title compound was prepared in substantially the same manner as described in Example 1, from 2,4-dimethoxyaniline and 2,3-dimethoxybenzoic acid, and was obtained as a tan solid, m.p. 256-258° C.; MS m/e 244 (M+H)⁺.

Analysis for: C₁₃H₉NO₄ Calc'd: C, 64.20; H, 3.73; N, 5.76 Found: C, 63.91; H, 3.98; N, 5.72

### Example 10

### 4-(6-HYDROXY-1,3-BENZOXAZOL-2-YL)BENZENE-1,2-DIOL

The title compound was prepared in substantially the same manner as described in Example 1, from 2,4-dimethoxyaniline and 3,4-dimethoxybenzoic acid, and was obtained as a white solid, m.p. 282-284° C.; MS m/e 242 (M−H)⁺.

Analysis for: C₁₃H₉NO₄ Calc'd: C, 64.20; H, 3.73; N, 5.76 Found: C, 63.57; H, 3.68; N, 5.63

### Example 11

### 2-(3-CHLORO-4-HYDROXYPHENYL)-1,3-BENZOXAZOL-6-OL

The title compound was prepared in substantially the same manner as described in Example 1, from 2,4-dimethoxyaniline and 3-chloro-4-methoxybenzoic acid, and was obtained as an off-white solid, m.p. 254-256° C.; MS m/e 262 (M+H)⁺.

Analysis for: C₁₃H₉NO₄ Calc'd: C, 64.20; H, 3.73; N, 5.76 Found: C, 63.57; H, 3.68; N, 5.63

### Example 12

### 2-(4-HYDROXYPHENYL)-1,3-BENZOXAZOL-5-OL

The title compound was prepared in substantially the same manner as described in Example 1, from 2,5-dimethoxyaniline and 4-methoxybenzoyl chloride, and was obtained as a light yellow solid, m.p. 264-267° C.; MS m/e 228 (M+H)⁺.

Analysis for: C₁₃H₉NO₃ Calc'd: C, 68.72; H, 3.99; N, 6.16 Found: C, 67.87; H, 4.05; N, 6.23

### Example 13

### 4-(5-HYDROXY-1,3-BENZOXAZOL-2-YL)BENZENE-1,3-DIOL

The title compound was prepared in substantially the same manner as described in Example 1, from 2,5-dimethoxyaniline and 2,4-dimethoxybenzoic acid, and was obtained as a white solid, m.p. greater than 300° C.; MS m/e 242 (M−H)⁺.

Analysis for: C₁₃H₉NO₄ Calc'd: C, 64.20; H, 3.73; N, 5.76 Found: C, 63.92; H, 3.74; N, 5.56

### Example 14

### 2-(4-HYDROXYPHENYL)-1,3-BENZOXAZOL-6-OL

The title compound was prepared in substantially the same manner as described in Example 1, from 2,4-dimethoxyaniline and 4-methoxybenzoyl chloride, and was obtained as a white solid, m.p. greater than 300° C.; MS m/e 226 (M−H)⁺.

Analysis for: C₁₃H₉NO₃ Calc'd: C, 68.72; H, 3.99; N, 6.16 Found: C, 68.09; H, 4.01; N, 6.05

### Example 15

### 4-(6-HYDROXY-1,3-BENZOXAZOL-2-YL)BENZENE-1,3-DIOL

The title compound was prepared in substantially the same manner as described in Example 1, from 2,4-dimethoxyaniline and 2,4-dimethoxybenzoic acid, and was obtained as a white solid, m.p. 293-296° C.; MS m/e 242 (M−H)⁺.

Analysis for: C₁₃H₉NO₄ Calc'd: C, 64.20; H, 3.73; N, 5.76 Found: C, 64.43; H, 3.77; N, 5.74

### Example 16

### 6-CHLORO-2-(3-FLUORO-4-HYDROXYPHENYL)-1,3-BENZOXAZOL-5-OL

### Step a) n-(4-chloro-2,5-dimethoxyphenyl)-3-fluoro-4-methoxybenzamide

The title compound was prepared in substantially the same manner as described in Example 1, Step a, from 4-chloro-2,5-dimethoxyaniline and 3-fluoro-4-methoxybenzoic acid, and was obtained as a white solid, m.p. 197-199° C.; MS m/e 340 (M+H)⁺.

Analysis for: C₁₆H₁₅CIFNO₄ Calc'd: C, 56.56; H, 4.45; N, 4.12 Found: C, 56.33; H, 4.35; N, 4.05

### Step b) N-(4-Chloro-2,5-dihydroxyphenyl)-3-fluoro-4-hydroxybenzamide

Boron trifluoride dimethyl sulfide complex (70 mL) was added into a mixture of N-(4-chloro-2,5-dimethoxyphenyl)-3-fluoro-4-methoxybenzamide (1.75 g, 5.15 mmol) and CH₂Cl₂ (35 mL). After stirring for 20 hours, the solvent and the excess reagent were evaporated under a nitrogen stream in the hood. The residue was taken into a mixture of ice and HCl (1N) and extracted with EtOAc. The organic layer was washed with HCl (1N) and dried over MgSO₄. Evaporation and purification by flash chromatography (CH₂Cl₂/hexanes/EtOAc 5/3/2, and AcOH 10 mL per 1 liter of the eluting solvent) gave a white solid (1.4 g, 91% yield, m.p. 254-256° C.); MS m/e 296 (M−H)⁺.

Analysis for: C₁₃H₉CIFNO₄ Calc'd: C, 52.46; H, 3.05; N, 4.71 Found: C, 51.98; H, 2.98; N, 4.56

### Step c) 6-Chloro-2-(3-fluoro-4-hydroxyphenyl)-1,3-benzoxazol-5-ol

The title compound was prepared in substantially the same manner as described in Example 1, step b, from N-(4-chloro-2,5-dihydroxyphenyl)-3-fluoro-4-hydroxybenzamide and pyridine hydrochloride, and was obtained as a white solid, m.p. 258-260° C.; MS m/e 278 (M−H)⁺.

Analysis for: C₁₃H₁₇CIFNO₃ Calc'd: C, 55.83; H, 2.52; N, 5.01 Found: C, 55.35; H, 2.59; N, 4.91

### Example 17

### 6-BROMO-2-(3-FLUORO-4-HYDROXYPHENYL)-1,3-BENZOXAZOL-5-OL

The title compound was prepared in substantially the same manner as described in Example 16, from 4-bromo-2,5-dimethoxyaniline and 3-fluoro-4-methoxybenzoic acid, and was obtained as a white solid, m.p. 224-226° C.; MS m/e 322 (M−H)⁺.

Analysis for: C₁₃H₁₇BrFNO₃ Calc'd: C, 48.18; H, 2.18; N, 4.32 Found: C, 48.69; H, 2.36; N, 4.59

### Example 18

### 6-CHLORO-2-(4-HYDROXYPHENYL)-1,3-BENZOXAZOL-5-OL

The title compound was prepared in substantially the same manner as described in Example 16, from 4-chloro-2,5-dimethoxyaniline and 4-methoxybenzoyl chloride, and was obtained as an off-white solid, m.p. 260-262° C.; MS m/e 260 (M−H)⁺.

Analysis for: C₁₃H₈CINO₃ Calc'd: C, 59.67; H, 3.08; N, 5.35 Found: C, 59.09; H, 3.06; N, 5.11

### Example 19

### 5-CHLORO-2-(4-HYDROXYPHENYL)-1,3-BENZOXAZOL-6-OL

The title compound was prepared in substantially the same manner as described in Example 16, from 5-chloro-2,4-dimethoxyaniline and 4-methoxybenzoyl chloride, and was obtained as an off-white solid, m.p. 254-256° C.; MS m/e 262 (M+H)⁺.

Analysis for: C₁₃H₈CINO₃ Calc'd: C, 59.67; H, 3.08; N, 5.35 Found: C, 59.40; H, 2.97; N, 5.22

### Example 20

### 7-BROMO-2-(4-HYDROXYPHENYL)-1,3-BENZOXAZOL-5-OL

### Step a) 2-Bromo-4-methoxy-6-nitrophenol

Bromine (16.0 g, 100 mmol) in acetic acid (20 mL) was added into a mixture of 4-methoxy-2-nitrophenol (16.9 g, 100 mmol), sodium acetate (16.4 g, 200 mmol) and acetic acid (100 mL). The reaction mixture was stirred for 30 mins. at room temperature, and then at 70° C. for 2 hours and poured into water (1.5 l) containing concentrated sulfuric acid (10 mL). The precipitated solid was filtered and crystallized from chloroform/hexane to give a brownish solid, m.p. 116-118° C.; MS m/e 246 (M−H)⁺.

Analysis for: C₇H₆BrNO₄ Calc'd: C, 33.90; H, 2.44; N, 5.65 Found: C, 34.64; H, 2.16; N, 5.43

### Step b) 2-Amino-6-bromo-4-methoxyphenol

Raney/Ni (2.5 g) was added into a solution of 2-bromo-4-methoxy-6-nitrophenol (8.8 g, 35.5 mmol) in EtOAc (100 mL). The mixture was shaken in a Parr apparatus under hydrogen at 25 psi for 2.5 hours. The reaction mixture was filtered through Celite® and concentrated under vacuum to give a gray solid (7.4 g, 96% yield; 95-97° C.); MS m/e 218 (M+H)⁺.

Analysis for: C₇H₈BrNO₂ Calc'd: C, 38.56; H, 3.70; N, 6.42 Found: C, 38.32; H, 3.77; N, 6.24

### Step c) 2-Bromo-4-methoxy-6-[(4-methoxybenzoyl)amino]phenyl-4-methoxybenzoate

Anhydrous pyridine (37.0 mL, 468.5 mmol) was added dropwise into a cold (0° C.) mixture (mechanically stirred) of 2-amino-6-bromo-4-methoxyphenol (20.0 g, 91.7 mmol), 4-methoxybenzoyl chloride (38.9 g, 229.0 mmol), and CH₂Cl₂ (250 mL). During the pyridine addition, a precipitate was formed. The reaction mixture was stirred for 30 mins. and then ethyl ether (250 mL) was added. The precipitated solids were filtered off and washed with ethyl ether. The solids were taken into water and stirred for 20 min. The solids were then filtered off and dried to give an off-white solid (42.5 g, 95% yield, m.p. 73-75° C.); MS m/e 484 (M−H)⁺.

Analysis for: C₂₃H₂₀BrNO₆ Calc'd: C, 56.80; H, 4.15; N, 2.88 Found: C, 56.50; H, 3.78; N, 2.83

### Step d) 7-Bromo-5-methoxy-2-(4-methoxyphenyl)-1,3-benzoxazole

Route a)

A suspension of 2-bromo-4-methoxy-6-[(4-methoxybenzoyl)amino]phenyl 4-methoxybenzoate (42.0 g, 86.4 mmol), p-toluenesulfonic acid monohydrate (32.8 g, 172.8 mmol) and anhydrous p-xylene (800 mL) was refluxed for 1 hour with continuous water removal (Dean-Stark Trap). The initial suspension turned into a brown solution at refluxing temperature. The reaction mixture was cooled to room temperature and washed with NaOH (2N). The organic layer was dried over MgSO₄. Evaporation and crystallization from acetone/ethyl ether gave an off-white solid (23.5 g, 82% yield, m.p. 139-141° C.); MS m/e 334 (M+H)⁺.

Analysis for: C₁₅H₁₂BrNO₃ Calc'd: C, 53.91; H, 3.62; N, 4.19 Found: C, 53.83; H, 3.37; N, 4.01

Route b)

A mixture of 2-amino-6-bromo-methoxyphenol (100 mg, 0.46 mmol), 4-methoxy-benzoic acid (77 mg, 0.5 mmol), and boric acid (31 mg, 0.5 mmol) in p-xylene (9 mL) was refluxed for 24 hours using a Dean-Stark water separator. The reaction mixture was cooled to room temperature, and concentrated under vacuum. The residual product was purified by flash chromatography (30% EtOAc/petroleum ether) to give a light pink solid (99 mg, 65% yield, m.p. 136-138° C.); MS m/e 334 (M+H)⁺.

Analysis for: C₁₅H₁₂BrNO₃ Calc'd: C, 53.91; H, 3.62; N, 4.19 Found: C, 53.78; H, 3.55; N, 4.01.

### Step e) 7-Bromo-2-(4-hydroxyphenyl)-1,3-benzoxazol-5-ol

Route a)

Boron tribromide (1M, 89.9 mL, 89.8 mmol) was added dropwise into a cold (−70° C.) suspension of 7-bromo-5-methoxy-2-(4-methoxyphenyl)-1,3-benzoxazole (10.0 g, 29.94 mmol) and CH₂Cl₂ (50 mL). The reaction mixture was allowed to warm up to room temperature. During the warming up period, the suspension turned into a dark solution. The reaction mixture was stirred at room temperature for 2 days and then poured slowly into cold (0° C.) ethyl ether (1000 mL). Methyl alcohol (200 mL) was added slowly into the new reaction mixture over a 20 mins. period. The reaction mixture was then poured into water (1.5 l). The organic layer was washed three times with water, and dried over MgSO₄. Evaporation and crystallization from acetone/ethyl ether/hexanes gave an off-white solid (8.4 g, 92% yield, m.p. 298-299° C.); MS m/e 306 (M+H)⁺.

Analysis for: C₁₃H₈BrNO₃ Calc'd: C, 51.01; H, 2.63; N, 4.58 Found: C, 50.96; H, 2.30; N, 4.42

Route b)

Boron tribromide (0.25 mL, 2.7 mmol) was added dropwise into a cold (−78° C.) mixture of 7-bromo-5-methoxy-2-(4-methoxyphenyl)-1,3-benzoxazole (130 mg, 0.39 mmol), and dichloromethane (1.5 mL). The reaction mixture was allowed to come gradually to room temperature and stirred for 1 hour. The reaction mixture was poured into ice and extracted with EtOAc. The organic extracts were washed with brine and dried over MgSO4. Evaporation and flash chromatography (30%-40% EtOAc/petroleum ether) gave (102 mg, 86% yield) of the product as a light pink solid, m.p. 295-298° C.; MS m/e 304 (M−H)⁺.

Analysis for: C₁₃H₈BrNO₃ Calc'd: C, 51.01; H, 2.63; N, 4.58 Found: C, 51.06; H, 2.77; N, 4.36.

### Example 21

### 7-BROMO-2-(3-FLUORO-4-HYDROXYPHENYL)-1,3-BENZOXAZOL-5-OL

### Step a) 2-Bromo-6-[(3-fluoro-4-methoxybenzoyl)amino]-4-methoxyphenyl 3-fluoro-4-methoxybenzoate

A mixture of 3-fluoro-4-methoxybenzoic acid (39.0 g, 229 mmol), thionyl chloride (100 mL), and N,N-dimethylformamide (0.5 mL) was refluxed for 1 hour. The volatiles were removed under vacuum. The solids were taken in benzene (twice) and the volatiles were removed under vacuum. The residue was dissolved in CH₂Cl₂ (100 mL) and added into a cold (0° C.) mixture (mechanically stirred) of 2-amino-6-bromo-4-methoxyphenol (20.0 g, 91.7 mmol) and CH₂Cl₂ (150 mL). Anhydrous pyridine (37.0 mL, 468.5 mmol) was added dropwise into the new reaction mixture. During the pyridine addition, a precipitate was formed. The reaction mixture was stirred for 30 mins. and then ethyl ether (250 mL) was added. The precipitated solids were filtered off and washed with ethyl ether. The solids were taken into water and stirred for 20 mins. The solids were then filtered off and dried to give an off-white solid (46.5 g, 97% yield, m.p. 184-186° C.); MS m/e 520 (M−H)⁺.

Analysis for: C₂₃H,₈BrF₂NO₆ Calc'd: C, 52.89; H, 3.47; N, 2.68 Found: C, 52.79; H, 3.23; N, 2.63

### Step b) 7-Bromo-2-(3-fluoro-4-methoxyphenyl)-5-methoxy-1,3-benzoxazole

A suspension of 2-bromo-6-[(3-fluoro-4-methoxybenzoyl)amino]-4-methoxyphenyl 3-fluoro-4-methoxybenzoate (46.0 g, 88.1 mmol), p-toluenesulfonic acid monohydrate (33.5 g, 177.2 mmol) and anhydrous p-xylene (1 l) was refluxed for 3 hours with continuous water removal (Dean Stark Trap). The initial suspension turned into a brown solution at refluxing temperature. The solids were filtered off and washed with ethyl ether. The solids were suspended in ethyl ether (200 mL), stirred for 10 mins., filtered off and dried to give a tan solid (25.1 g, m.p. 175-177° C.). The ethyl ether layer was concentrated to 20 mL and 2.5 g of additional product was obtained (90% overall yield). MS m/e 352 (M+H)⁺.

Analysis for: C₁₅H₁₁BrFNO₃ Calc'd: C, 51.16; H, 3.15; N, 3.98 Found: C, 51.10; H, 2.92; N, 3.89

step c) 7-bromo-2-(3-fluoro-4-hydroxyphenyl)-1,3-benzoxazol-5-ol

The title compound was prepared in substantially the same manner as described in Example 20, Step e, and was obtained as a white solid, m.p. 265-267° C.; MS m/e 332 (M−H)⁺.

Analysis for: C₁₃H₇BrFNO₃ Calc'd: C, 48.18; H, 2.18; N, 4.32 Found: C, 48.19; H, 2.29; N, 4.19

### Example 22

### 7-BROMO-2-(2-FLUORO-4-HYDROXYPHENYL)-1,3-BENZOXAZOL-5-OL

### Step a) 2-Fluoro-4-methoxybenzoic acid

Into a warm (55° C.) mixture of Ag₂O (13.5 g, 58.4 mmol), NaOH (19.5 g, 487 mmol) and water (200 mL), was added 2-fluoro-4-methoxybenzaldehyde (15 g, 97.4 mmol). The reaction mixture was stirred for 1 hour, filtered off and the precipitated solids were washed with hot water (10 mL). The filtrate was added slowly into cold (0° C.) HCl (5N) with vigorous stirring. The precipitated solid was filtered, washed with water and dried to give a white solid (13.6 9, 82% yield, m.p. 194-196° C.); MS m/e 169 (M−H)⁺.

Analysis for: C₈H₇FO₃ Calc'd: C, 56.48; H, 4.15 Found: C, 56.12; H, 4.12

### Step b) 7-Bromo-2-(2-fluoro-4-hydroxyphenyl)-1,3-benzoxazol-5-ol

The title compound was prepared in substantially the same manner as described in Example 21, from 2-fluoro-4-methoxybenzoic acid, and was obtained as a white solid, m.p. 248-250° C.; MS m/e 324 (M+H)⁺.

Analysis for: C₁₃H₇BrFNO₃ Calc'd: C, 48.18; H, 2.18; N, 4.32 Found: C, 47.89; H, 1.95; N, 4.18

### Example 23

### 7-BROMO-2-(2,3-DIFLUORO-4-HYDROXYPHENYL)-1,3-BENZOXAZOL-5-OL

### Step a) Methyl 2,3-difluoro-4-methoxybenzoate

Iodomethane (10.7 mL, 172.5 mmol) was added into a mixture of 2,3-difluoro-4-hydroxybenzoic acid (10.0 g, 57.5 mmol), lithium carbonate (12.7 g, 172.5 mmol) and N,N-dimethylformamide (100 mL). The reaction mixture was stirred at 40° C. for 12 h, and then poured into water and extracted with EtOAc. The organic extracts were dried over MgSO₄. Evaporation and purification by flash chromatography (hexanes/EtOAc 5/1) gave a white solid (10.2 g, 88% yield, m.p. 66-68° C.); MS m/e 203 (M+H)⁺.

Analysis for: CgH₈F₂O₃ Calc'd: C, 53.47; H, 3.99 Found: C, 53.15; H, 3.83

### Step b) 2,3-Difluoro-4-methoxybenzoic acid

Sodium hydroxide (2N, 50 mL) was added into a mixture of methyl 2,3-difluoro-4-methoxybenzoate (10.0 g, 49.5 mmol), THF (100 mL) and MeOH (100 mL). The reaction mixture was stirred at room temperature for 6 hours, and acidified with HCl (2N). The precipitated solid was filtered off, washed with water and dried to give a white solid (8.9 g, 96% yield, m.p. 194-196° C.); MS m/e 187 (M−H)⁺.

Analysis for: C₈H₆F₂O₃ Calc'd: C, 51.08; H, 3.21 Found: C, 50.83; H, 2.92

### Step c) 7-Bromo-2-(2,3-difluoro-4-hydroxyphenyl)-1,3-benzoxazol-5-ol

The title compound was prepared in substantially the same manner as described in Example 21, from 2,3-difluoro-4-methoxybenzoic acid, and was obtained as a white solid, m.p. 258-260° C.; MS m/e 342 (M+H)⁺.

Analysis for: C₁₃H₆BrF₂NO₃ Calc'd: C, 45.64; H, 1.77; N, 4.09 Found: C, 45.33; H, 1.62; N, 4.02

### Example 24

### 2-(3-FLUORO-4-HYDROXYPH ENYL)-7-VI NYL-1,3-BENZOXAZOL-5-OL

Route a)

### Step a) 7-Bromo-5-{[tert-butyl(dimethyl)silyl]oxy}-2-(4-{[tert-butyl(dimethyl)silyl]oxy}-3-fluorophenyl)-1,3-benzoxazole

tert-Butyl(chloro)dimethylsilane (23.2 g, 154 mmol) was added portionwise into a mixture of 7-bromo-2-(3-fluoro-4-hydroxyphenyl)-1,3-benzoxazol-5-ol (16.6 9, 51.4 mmol), imidazole (17.5 g, 257 mmol), N,N-dimethylpyridin-4-amine (1.0 g, 8.1 mmol) and DMF (300 mL). The reaction mixture was stirred for 3 hours, poured into water and extracted with ethyl ether. The organic extracts were dried over MgSO₄. Evaporation and purification by flash chromatography (hexanes /EtOAc 50/1) gave a white solid (27.5 g, 97% yield, m.p. 98-99° C.); MS m/e 552 (M+H)⁺.

Analysis for: C₂₅H₃₅BrFNO₃Si₂ Calc'd: C, 54.34; H, 6.38; N, 2.53 Found: C, 54.06; H, 6.52; N, 2.24

### Step b) 5-{[tert-Butyl(dimethyl)silyl]oxy}-2-(4-{[tert-butyl(dimethyl)silyl]oxy}-3-fluorophenyl)-7-vinyl-1,3-benzoxazole

Dichlorobis(tri-otolylphosphine)palladium (II) (0.63 g, 0.79 mmol) was added into a mixture of 7-bromo-5-{[tert-butyl(dimethyl)silyl]oxy}-2-(4-{[tert-butyl(dimethyl)silyl]oxy}-3-fluorophenyl)-1,3-benzoxazole (14.7 g, 26.6 mmol), tributyl(vinyl)tin (10.5 g, 33.25 mmol) and p-xylene (85 mL). The reaction mixture was stirred at 90° C. for 24 hours, cooled to room temperature, diluted with ethyl ether (100 mL) and treated with activated carbon. The reaction mixture was filtered through MgSO₄ and concentrated. Purification by flash chromatography (hexanes/EtOAc 50/1) gave a white solid (11.8 g, 89% yield, m.p. 93-95° C.); MS m/e 500 (M+H)⁺.

Analysis for: C₂₇H₃₈FNO₃Si₂ Calc'd: C, 64.89; H, 7.66; N, 2.80 Found: C, 64.59; H, 7.70; N, 2.73

### Step c) 2-(3-Fluoro-4-hydroxyphenyl)-7-vinyl-1,3-benzoxazol-5-ol

Hydrofluoric acid (48 wt. % in water, 1 mL) was added into a solution of 5-{[tert-butyl(dimethyl)silyl]oxy}-2-(4-{[tert-butyl(dimethyl)silyl]oxy}-3-fluorophenyl)-7-vinyl-1,3-benzoxazole (1.5 g, 3.0 mmol), THF (6 mL) and acetonitrile (3 mL). The reaction mixture was stirred at 65° C. for 8 hours, and then poured into water. The precipitated solid was filtered off and dried. Crystallization of the product from acetone/ethyl ether gave a white solid (0.72 g, 81% yield, m.p. 249-251° C.); MS m/e 272 (M+H)⁺.

Analysis for: C₁₅H₁₀FNO₃ Calc'd: C, 66.42; H, 3.72; N, 5.16 Found: C, 66.31; H, 3.85; N, 4.96

Route b)

2-(3-Fluoro-4-hydroxyphenyl)-7-vinyl-1,3-benzoxazol-5-ol.

Dichlorobis(tri-otolylphosphine)palladium (II) (0.87 g, 1.1 mmol) was added into a mixture of 7-bromo-2-(3-fluoro-4-hydroxyphenyl)-1,3-benzoxazol-5-ol (7.16 g, 22.1 mmol), tributyl(vinyl)tin (10.5 g, 33.25 mmol) and ethylene glycol diethyl ether (65 mL). The reaction mixture was stirred at 115° C. for 48 hours, cooled to room temperature and treated with activated carbon. The reaction mixture was filtered through MgSO₄ and concentrated. Purification by flash chromatography, on acidic silica gel (hexanes/EtOAc/CH₂Cl₂ 1/1/1), gave a white solid (4.35 g, 72% yield, m.p. 250-252° C.); MS m/e 272 (M+H)⁺.

Analysis for: C₁₅H₁₀FNO₃ Calc'd: C, 66.42; H, 3.72; N, 5.16 Found: C, 66.03; H, 3.68; N, 5.09

Route c)

### Step a) 4-[5-(Acetyloxy)-7-bromo-1,3-benzoxazol-2-yl]-2-fluorophenyl acetate

Acetic anhydride (1.0 mL, 9.95 mmol) was added into a cold (0° C.) solution of 7-bromo-2-(3-fluoro-4-hydroxyphenyl)-1,3-benzoxazol-5-ol (1.24 g, 3.8 mmol), N,N-dimethylpyridin-4-amine (1.1 g, 9.18 mmol) and 1,4-dioxane (13 mL). The reaction mixture was allowed to warm up to room temperature and stirred for 20 hours. Water (50 mL) was added to the reaction mixture extracted with EtOAc and dried over MgSO₄. Evaporation and crystallization from EtOAc/hexane gave an off-white solid (0.87 g, 56% yield); MS m/e 408 (M+H)⁺.

Analysis for: C₁₇H₁₁BrFNO₅ Calc'd: C, 50.02; H, 2.72; N, 3.43 Found: C, 49.58; H, 2.59; N, 3.37

### Step b) 2-[4-(Acetyloxy)-3-fluorophenyl]-7-vinyl-1,3-benzoxazol-5-yl acetate

Dichlorobis(tri-otolylphosphine)palladium (II) (46 mg, 0.06 mmol) was added into a mixture of 4-[5-(acetyloxy)-7-bromo-1,3-benzoxazol-2-yl]-2-fluorophenyl acetate (0.8 g, 1.98 mmol), tributyl(vinyl)tin (0.9 g, 2.8 mmol) and p-xylene (9 mL). The reaction mixture was stirred at 130° C. for 5 hours, cooled to room temperature, diluted with ethyl ether (10 mL) and treated with activated carbon. The reaction mixture was filtered through MgSO₄ and concentrated. Purification by flash chromatography (hexanes/EtOAc 5/1) gave a white solid (0.4 g, 56% yield, m.p. 154-156° C.); MS m/e 356 (M+H)⁺.

Analysis for: C₁₉H₁₄FNO₅ Calc'd: C, 64.23; H, 3.97; N, 3.94 Found: C, 63.94; H, 3.78; N, 3.76

### Step c) 2-(3-Fluoro-4-hydroxyphenyl)-7-vinyl-1,3-benzoxazol-5-ol

Potassium carbonate (55 mg) was added into a solution of 2-[4-(acetyloxy)-3-fluorophenyl]-7-vinyl-1,3-benzoxazol-5-yl acetate (0.14 g, 0.39 mmol) and 1,4-dioxane (3 mL). The reaction mixture was stirred at 90° C. for 1 hour, poured into water, acidified with HCl (2N) and extracted with EtOAc. The organic extracts were dried over MgSO₄. Evaporation and crystallization from EtOAc/hexanes, gave a white solid (0.06 g, 46% yield, m.p. 250-252° C.); MS m/e 272 (M+H)⁺.

Analysis for: C₁₅H₁₀FNO3 Calc'd: C, 66.42; H, 3.72; N, 5.16 Found: C, 66.32; H, 3.47; N, 5.18

### Example 25

### 2-(2-FLUORO-4-HYDROXYPHENYL)-7-VINYL-1,3-BENZOXAZOL-5-OL

The title compound was prepared in substantially the same manner as described in Example 24, Route a), from 7-bromo-2-(2-fluoro-4-hydroxyphenyl)-1,3-benzoxazol-5-ol, and was obtained as a white solid, m.p. 274-275° C.; MS m/e 272 (M+H)⁺.

Analysis for: C₁₅H₁₀FNO₃ Calc'd: C, 66.42; H, 3.72; N, 5.16 Found: C, 66.18; H, 3.47; N, 4.97

### Example 26

### 2-(2,3-DIFLUORO-4-HYDROXYPHENYL)-7-VINYL-1,3-BENZOXAZOL-5-OL

The title compound was prepared in substantially the same manner as described in Example 24, Route b), from 7-bromo-2-(2,3-difluoro-4-hydroxyphenyl)-1,3-benzoxazol-5-ol, and was obtained as an off-white solid, m.p. 276-278° C.; MS m/e 290 (M+H)⁺.

Analysis for: C₁₅H₉F₂NO₃ Calc'd: C, 62.29; H, 3.14; N, 4.84 Found: C, 61.90; H, 3.05; N, 4.52

### Example 27

### 2-(4-HYDROXYPHENYL)-7-VINYL-1,3-BENZOXAZOL-5-OL

The title compound was prepared in substantially the same manner as described in Example 24, Route b), from 7-bromo-2-(4-hydroxyphenyl)-1,3-benzoxazol-5-ol, and was obtained as a white solid, m.p. 249-250° C.; MS m/e 254 (M+H)⁺.

Analysis for: C₁₅H₁₁NO₃ Calc'd: C, 70.99; H, 4.39; N, 5.52 Found: C, 70.75; H, 4.34; N, 5.46

### Examples 28 and 29

### 4-BROMO-2-(3-FLUORO-4-HYDROXYPHENYL)-7-VINYL-1,3-BENZOXAZOL-5-OL (EX. 28) AND 4,6-DIBROMO-2-(3-FLUORO-4-HYDROXYPHENYL)-7-VINYL-1,3-BENZOXAZOL-5-OL (EX. 29).

N-Bromosuccinimide (0.49 g, 2.77 mmol) was added into a mixture of 2-(3-fluoro-4-hydroxyphenyl)-7-vinyl-1,3-benzoxazol-5-ol (0.75 g, 2.77 mmol) and acetonitrile (30 mL). The reaction mixture was stirred at room temperature for 16 hours, poured into water and extracted with EtOAc. The organic extracts were dried over MgSO₄. Evaporation and purification by flash chromatography (hexanes/EtOAc/CH₂Cl₂ 2/1/1) gave Ex. 28 as a white solid (0.45 g, m.p. 226-228° C.); MS m/e 349 (M+H)⁺.

Analysis for: C₁₅H₉BrNO₃ Calc'd: C, 51.45; H, 2.59; N, 4.00 Found: C, 51.08; H, 2.40; N, 3.90;

and Ex. 29 as a white solid (0.18 g, m.p. 272-274° C.); MS m/e 428 (M+H)⁺.

Analysis for: C₁₅H₈Br₂NO₃ Calc'd: C, 41.99; H, 1.88; N, 3.26 Found: C, 42.25; H, 1.90; N, 3.14

### Example 30

### 7-(1,2-DIBROMOETHYL)-2-(4-HYDROXYPHENYL)-1,3-BENZOXAZOL-5-OL

### Step a) 5-Methoxy-2-(4-methoxyphenyl)-7-vinyl-1,3-benzoxazole

The title compound was prepared in substantially the same manner as described in Example 24, Route c), Step b) from 7-bromo-5-methoxy-2-(4-methoxyphenyl)-1,3-benzoxazole, and was obtained as a white solid, MS m/e 282 (M+H)⁺.

Analysis for: C₁₇H₁₅NO₃ Calc'd: C, 72.58; H, 5.37; N, 4.98 Found: C, 72.33; H, 5.26; N, 4.72

### Step b) 7-(1,2-Dibromoethyl)-2-(4-hydroxyphenyl)-1,3-benzoxazol-5-ol

Boron tribromide (0.85 mL, 8.95 mmol) was added dropwise into a cold (−78° C.) mixture of 5-methoxy-2-(4-methoxyphenyl)-7-vinyl-1,3-benzoxazole (0.31 g, 1.12 mmol) and CH₂Cl₂ (4 mL). The reaction mixture was allowed to warm up to room temperature. After stirring for 18 hours at room temperature the reaction mixture was slowly poured into cold (0° C.) ethyl ether (20 mL). Methyl alcohol (10 mL) was then slowly added into the reaction mixture. The new reaction mixture was washed with water (three times) and dried over MgSO₄. Evaporation and purification by flash chromatography (hexanes/EtOAc 3/1) gave a light yellow solid (0.27 g, 59% yield, m.p. 175-177° C.); MS m/e 412 (M+H)⁺.

Analysis for: C₁₅H₁₁Br₂NO₃ Calc'd: C, 43.62; H, 2.68; N, 3.39 Found: C, 43.85; H, 2.44; N, 3.33

### Example 31

### 7-(1-BROMOVINYL)-2-(4-HYDROXYPHENYL)-1,3-BENZOXAZOL-5-OL

1,8-Diazabicyclo[5.4.0]undec-7-ene (0.25g, 1.65mmol) was added into a solution of 7-(1,2-dibromoethyl)-2-(4-hydroxyphenyl)-1,3-benzoxazol-5-ol (0.4 g, 0.96 mmol) and acetonitrile (4 mL). The reaction mixture was stirred for 24 hours, poured into cold (0° C) HCl (1N, 10 mL) and extracted with EtOAc. The organic extracts were dried over MgSO₄. Evaporation and purification by flash chromatography (CH₂Cl₂ /hexanes/isopropyl alcohol 15/5/1) gave a white solid (185 mg, 58% yield, m.p. 228-230° C.); MS m/e 332 (M+H)⁺.

Analysis for: C₁₅H₁₀BrNO₃ Calc'd: C, 54.24; H, 3.03; N, 4.22 Found: C, 54.27; H, 2.94; N, 4.20

### Example 32

### 7-(1-BROMOVINYL)-2-(2-FLUORO-4-HYDROXYPHENYL)-1,3-BENZOXAZOL-5-OL

The title compound was prepared in substantially the same manner as described in Examples 29-30, from 7-bromo-2-(2-fluoro-4-methoxyphenyl)-5-methoxy-1,3-benzoxazole, and was obtained as an off-white solid, m.p. 235-237° C.; MS m/e 350 (M+H)⁺.

Analysis for: C₁₅H₉BrFNO₃ Calc'd: C, 51.45; H, 2.59; N, 4.00 Found: C, 51.63; H, 2.38; N, 3.98

### Example 33

### 7-(1-BROMOVINYL)-2-(2,3-DIFLUORO-4-HYDROXYPHENYL)-1,3-BENZOXAZOL-5-OL

The title compound was prepared in substantially the same manner as described in Examples 29-30, from 7-bromo-2-(2,3-difluoro-4-methoxyphenyl)-5-methoxy-1,3-benzoxazole, and was obtained as an off-white solid, m.p. 240-242° C.; MS m/e 366 (M−H)⁺.

Analysis for: C₁₅H₈BrF₂NO₃ Calc'd: C, 48.94; H, 2.19; N, 3.80 Found: C, 49.63; H, 2.33; N, 3.61

### Example 34

### 7-ALLYL-2-(3-FLUORO-4-HYDROXYPHENYL)-1,3-BENZOXAZOL-5-OL

The title compound was prepared in substantially the same manner as described in Example 24, Route c, Step b, from 7-bromo-2-(3-fluoro-4-methoxyphenyl)-5-methoxy-1,3-benzoxazole, allyltributyltin and dichlorobis(tri-o-tolylphosphine)palladium, followed by demethylation according to Example 20, Step e. The desired product was obtained as a light pink solid, m.p. 169-171° C.; MS m/e 284 (M−H)⁺.

Analysis for: C₁₆H₁₂FNO₃ Calc'd: C, 67.37; H, 4.24; N, 4.91 Found: C, 67.37; H, 4.16; N, 4.66

### Example 35

### 7-ETHYNYL-2-(4-HYDROXYPHENYL)-1,3-BENZOXAZOL-5-OL

Tetrakis(triphenylphosphine)palladium(0) (52 mg, 0.045 mmol) was added into a mixture of 7-bromo-5-methoxy-2-(4-methoxyphenyl)-1,3-benzoxazole (0.3 g, 0.9 mmol), copper(I) iodide (17.1 mg, 0.09 mmol), ethynyl(trimethyl)silane (0.2 g mg, 2 mmol) and triethylamine (12 mL). The reaction mixture was stirred at 110° C. for 4 hours, poured into aqueous ammonium chloride and extracted with EtOAc/THF (1/1). The organic extracts were dried over MgSO₄. Evaporation and purification by flash chromatography (hexanes/EtOAc 6/1) gave an off-white solid (0.27 g, 85% yield). The product was dissolved in CH₂Cl₂ (2 mL), cooled to −78° C. and boron tribromide (0.6 mL) was added dropwise. The reaction mixture was allowed to warm up to room temperature. After stirring for 18 hours at room temperature, the mixture was slowly poured into cold (0° C.) ethyl ether (10 mL). Methyl alcohol (3 mL) was then slowly added into the reaction mixture. The new reaction mixture was washed with water (three times) and dried over MgSO₄. Evaporation and purification by flash chromatography (hexanes/EtOAc 3/1) gave a yellow solid (86 mg, 38% yield, m.p. 229-231° C.); MS m/e 252 (M+H)⁺.

Analysis for: C₁₅H₉NO₃ Calc'd: C, 71.71; H, 3.61; N, 5.58 Found: C, 71.39; H, 3.49; N, 5.32

### Example 36

### 2-(4-HYDROXYPHENYL)-7-PROPYL-1,3-BENZOXAZOL-5-OL

Tetrakis(triphenylphosphine)palladium(0) (70 mg, 0.06 mmol) was added into a mixture of 7-bromo-5-methoxy-2-(4-methoxyphenyl)-1,3-benzoxazole (0.4 g, 1.2 mmol), bromo(propyl)zinc (0.5 M in THF, 3.6 mL, 1.8 mmol), and THF (4 mL). The reaction mixture was stirred at room temperature for 48 hours, poured into HCl (1 N) and extracted with EtOAc. The organic extracts were dried over MgSO₄. Evaporation and purification by flash chromatography (hexanes/EtOAc 6/1) gave an off-white solid (0.14 g). The product was dissolved in CH₂Cl₂ (2 mL), cooled to −78° C. and boron tribromide (0.35 mL) was added dropwise. The reaction mixture was allowed to warm up to room temperature. After stirring for 18 hours at room temperature, the reaction mixture was slowly poured into cold (0° C.) ethyl ether (10 mL). Methyl alcohol (3 mL) was then slowly added into the reaction mixture. The new reaction mixture was washed with water (three times) and dried over MgSO₄. Evaporation and purification by flash chromatography (hexanes/EtOAc 4/1) gave a white solid (90 mg, 27% yield, m.p. 110-112° C.); MS m/e 270 (M+H)⁺.

Analysis for: C₁₆H₁₅NO₃ Calc'd: C, 71.36; H, 5.61; N, 5.20 Found: C, 71.02; H, 5.58; N, 4.94

### Example 37

### 7-BUTYL-2-(4-HYDROXYPHENYL)-1,3-BENZOXAZOL-5-OL

### Example 38

### 7-CYCLOPENTYL-2-(4-HYDROXYPHENYL)-1,3-BENZOXAZOL-5-OL

The title compound was prepared in substantially the same manner as described in Example 35, from 7-bromo-5-methoxy-2-(4-methoxyphenyl)-1,3-benzoxazole and bromo(cyclopentyl)zinc. The desired product was obtained as a white solid, m.p. 220-222° C.; MS m/e 296 (M+H)⁺.

Analysis for: C₁₈H₁₇NO₃ Calc'd: C, 73.20; H, 5.80; N, 4.74 Found: C, 73.05; H, 5.74; N, 4.59

### Example 39

### ETHYL 5-HYDROXY-2-(4-HYDROXYPHENYL)-1,3-BENZOXAZOLE-7-CARBOXYLATE

### Step a) 7-Bromo-5-{[tert-butyl(dimethyl)silyl]oxy}-2-(4-{[tert-butyl(dimethyl)silyl]oxy}phenyl)-1,3-benzoxazole

The title compound was prepared in substantially the same manner as described in Example 24, Route a, Step a, from 7-bromo-5-methoxy-2-(4-methoxyphenyl)-1,3-benzoxazole and tert-butyl(chloro)dimethylsilane. The desired product was obtained as a white solid, m.p. 90-91° C.; MS m/e 534 (M+H)⁺.

Analysis for: C₂₅H₃₆BrNO₃Si₂ Calc'd: C, 56.16; H, 6.79; N, 2.62 Found: C, 55.66; H, 6.86; N, 2.68

### Step b) Ethyl 5-hydroxy-2-(4-hydroxyphenyl)-1,3-benzoxazole-7-carboxylate

n-Butyllithium (2.5 M, 0.3 mL, 0.75 mmol) was added dropwise into a cold (0° C.) solution of 7-bromo-5-{[tert-butyl(dimethyl)silyl]oxy}-2-(4-{[tert-butyl(dimethyl)silyl]oxy}phenyl)-1,3-benzoxazole (0.4 g, 0.75 mmol) and THF (4 mL). The reaction mixture was allowed to warm up to 40° C., and then stirred for 2 hours. [(Cyanocarbonyl)oxy]ethane (84 mg) in THF (1 ML) was added into the reaction mixture and the reaction mixture was allowed to warm up to 0° C. and stirred for 1 hour. The reaction was quenched with aqueous ammonium chloride, extracted with EtOAc, and dried over MgSO₄. Evaporation and purification by flash chromatography (hexanes/CH₂Cl₂/isopropyl alcohol 18/2/1) gave a colorless oil (340 mg). The product was dissolved in THF (3.5 mL) and treated with tetrabutylammonium fluoride (1M in THF, 1.4 mL). The reaction mixture was stirred for 30 mins., poured into HCl (1N) and extracted with EtOAc. The organic extracts were dried over MgSO₄. Evaporation and purification by flash chromatography (hexanes/CH₂Cl₂/isopropyl alcohol 5/2/1) gave a white solid (119 mg, 53% yield, m.p. 305-307° C.); MS m/e 300 (M+H)⁺.

Analysis for: C₁₆H₁₃NO₅ Calc'd: C, 64.21; H, 4.38; N, 4.68 Found: C, 64.04; H, 4.43; N, 4.40

### Example 40

### 2-(4-HYDROXYPHENYL)-7-PHENYL-1,3-BENZOXAZOL-5-OL

### Step a) 5-Methoxy-2-(4-methoxyphenyl)-7-phenyl-1,3-benzoxazole

7-Bromo-5-methoxy-2-(4-methoxyphenyl)-1,3-benzoxazole (200 mg, 0.60 mmol) and tetrakis(triphenylphosphine)palladium(0) (63 mg, 0.03 mmol) were dissolved in toluene (5 mL) and stirred for 10 mins. at room temperature under a nitrogen atmosphere. Benzene boronic acid (110 mg, 0.90 mmol) was added, followed by aqueous sodium carbonate (2 M, 1.5 mL) and ethanol (2 mL). The reaction mixture was refluxed for 12 hours, diluted with water and extracted with EtOAc. The organic extracts were dried over MgSO₄. Evaporation and purification by flash chromatography (20%-40% EtOAc/petroleum ether) gave the title compound as a light pink solid, mp 92° C.; MS m/e 332 (M+H)⁺.

Analysis for: C₂₁H₁₇NO₃ Calcd: C, 76.12; H, 5.17; N, 4.23 Found: C, 75.86; H, 5.08; N, 4.07

### Step b) 2-(4-Hydroxyphenyl)-7-phenyl-1,3-benzoxazol-5-ol

The title compound was prepared according to the procedure of Example 20, Step e (Route b), and was obtained as a purple solid, m.p. 255-258° C.; MS m/e 302 (M−H)⁺.

Analysis for: C₁₉H₁₃NO₃×0.25 H₂O Calcd: C, 74.14; H, 4.42; N, 4.55 Found: C, 73.81; H, 4.40; N, 4.35

### Example 41

### 5-HYDROXY-2-(4-HYDROXYPHENYL)-1,3-BENZOXAZOL-7-CARBONITRILE

### Step a) 5-Methoxy-2-(4-methoxyphenyl)-1,3-benzoxazole-7-carbonitrile

A solution of 7-bromo-5-methoxy-2-(4-methoxyphenyl)-1,3-benzoxazole (200 mg, 0.60 mmol) in anhydrous N,N dimethylformamide (1.5 mL) was stirred and heated to reflux under dry nitrogen with copper(I) cyanide (80 mg, 0.90 mmol) for 4 hours. The reaction mixture was cooled and poured into an excess of aqueous ethylenediaminetetraacetic acid. Isolation of the crude product gave the nitrile (164 mg, 98% yield) as tan needles from 30% EtOAc/petroleum ether; m.p. 180-183° C.; MS m/e 281 (M+H)⁺.

Analysis for C₁₆H₁₂N₂O₃×0.2 H₂O Calcd: C, 66.84; H, 4.48; N, 9.74 Found: C, 66.63; H, 4.33; N, 9.60

### Step b) 5-Hydroxy-2-(4-Hydroxyphenyl)-1,3-benzoxazol-7-carbonitrile

The title compound was prepared according to the procedure of Example 20, Step e (Route b), and was obtained as a light pink solid, mp 297-303° C.; MS m/e 253 (M+H)⁺. Analysis for: C₁₄H₈N₂O₃×0.5 H₂O Calcd: C, 64.37; H, 3.47; N, 10.72 Found: C, 64.44; H, 3.49; N, 9.92

### Example 42

### 5-HYDROXY-2-(4-HYDROXYPHENYL)-1,3-BENZOXAZOL-7-CARBOXAMIDE

The title compound was isolated as a minor product from the reaction of Example 40, Step b, as a light tan solid, m.p. 325° C.; MS m/e 271 (M+H)⁺.

Analysis for: C₁₄H₁₀N₂O₄×0.5 H₂O Calcd: C, 60.22; H, 3.97; N, 10.03 Found: C, 59.71; H, 3.91; N, 9.84

### Example 43

### 2-(4-HYDROXYPHENYL)-7-METHOXY-1,3-BENZOXAZOL-5-OL

A mixture of 7-bromo-2-(4-hydroxyphenyl)-1,3-benzoxazol-5-ol (100 mg, 0.33 mmol) and copper(I) bromide (56 mg, 0.39 mmol) in anhydrous N,N dimethylformamide (1.5 mL) was stirred with freshly prepared sodium methoxide (15 wt % in methanol, 1 ml) and heated to 120° C. for 4 hours. The reaction mixture was cooled and diluted with HCl (1 N, 5 ml). Isolation of the crude product with ethyl acetate followed by flash chromatography (40% -50% EtOAc/petroleum ether) gave the title compound as an off-white solid (50 mg, 60% yield, mp 225-228° C.); MS m/e 258 (M+H)⁺.

Analysis for C₁₄H₁₁NO₄×0.75 H₂O Calcd: C, 62.11; H, 4.65; N, 5.17 Found: C, 62.53; H, 4.73; N, 5.02.

### Example 44

### 7-ETHYL-2-(4-HYDROXYPHENYL)-1,3-BENZOXAZOL-5-OL

### Step a) 7-Ethyl-5-methoxy-2-(4-methoxyphenyl)-1,3-benzoxazole

n-Butyllithium (2.5 N, 0.43 mL, 1.08 mmol) was added dropwise into a cold (−78° C.) mixture of 7-bromo-5-methoxy-2-(4-methoxyphenyl)-1,3-benzoxazole (300 mg, 0.90 mmol) and THF (2 mL). The reaction mixture was allowed to stir for 0.5 hours. lodoethane (0.14 mL, 1.8 mmol) was added dropwise into the reaction mixture. The reaction mixture was allowed to warm to room temperature and stirred for 2 hours. The reaction was quenched with aqueous ammonium chloride, poured into water, and extracted with EtOAc. The organic extracts were washed with brine and dried over MgSO₄. Evaporation and flash chromatography (20% EtOAc/petroleum ether) gave the product (231 mg, 91% yield) as a light brown solid: m.p. 85° C.; MS m/e 284 (M+H)⁺.

Analysis for: C₁₇H₁₇NO₃×0.2 H₂O Calc'd: C, 70.28; H, 6.17; N, 4.94. Found: C, 70.12; H, 5.74; N, 4.82.

### Step b) 7-Ethyl-2-(4-hydroxyphenyl)-1,3-benzoxazol-5-ol

The title compound was prepared according to the procedure of Example 20, Step e (Route b), and was obtained as a light brown solid (98% yield), m.p. 110-115° C.; MS m/e 256 (M+H)⁺.

### Example 45

### 7-ETHYL-2-(2-ETHYL-4-HYDROXYPHENYL)-1,3-BENZOXAZOL-5-OL

### Step a) 7-Ethyl-5-methoxy-2-(2-ethyl-4-methoxyphenyl)-1,3-benzoxazole

The title compound was prepared according to the procedure of Example 43, Step a, employing two equivalents of n-butyllithium, and the crude product was used directly in the next step.

### Step b) 7-Ethyl-2-(2-ethyl-4-hydroxyphenyl)-1,3-benzoxazol-5-ol

The title compound was prepared from 7-ethyl-5-methoxy-2-(2-ethyl-4-methoxyphenyl)-1,3-benzoxazole according to the procedure of Example 20, Step e (Route b), and was obtained as a gray solid (87% yield); MS m/e 284 (M+H)⁺.

### Example 46

### 5-HYDROXY-2-(4-HYDROXYPHENYL)-1,3-BENZOXAZOLE-7-CARBALDEHYDE

### Step a) 5-Methoxy-2-(4-methoxyphenyl)-1,3-benzoxazole-7-carbaldehyde

The title compound was prepared according to the procedure of Example. 43, Step a, employing N-methylformanilide as the electrophile to give a light orange solid (94%, m.p. 153-155° C.); MS m/e 284 (M+H)⁺.

Analysis for: C₁₆H₁₃NO₄ Calc'd: C, 67.84; H, 4.63; N, 4.94 Found: C, 67.58; H, 4.53; N, 4.75

### Step b) 5-Hydroxy-2-(4-hydroxyphenyl)-1,3-benzoxazole-7-carbaldehyde

The title compound was prepared from 5-methoxy-2-(4-methoxyphenyl)-1,3-benzoxazole-7-carbaldehyde according to the procedure of Example 20, Step e (Route b) and was obtained as a dark yellow solid (99% yield, m.p. 273-275° C.); MS m/e 256 (M+H)⁺.

Analysis for: C₁₄H₉NO₄×0.25 H₂O Calcd.: C, 64.74; H, 3.69; N, 5.39 Found: C, 64.32; H, 3.59; N, 5.18.

### Example 47

### 7-(HYDROXYMETHYL)-2-(4-HYDROXYPHENYL)-1,3-BENZOXAZOL-5-OL

### Step a) 5-Methoxy-7-(hydroxymethyl)-2-(4-methoxyphenyl)-1,3-benzoxazole

Sodium borohydride (66.8 mg, 1.76 mmol) was added into a solution of 5-methoxy-2-(4-methoxyphenyl)-1,3-benzoxazole-7-carbaldehyde (250 mg, 0.88 mmol) in anhydrous MeOH (8 mL) at 0° C. The reaction mixture was stirred for 30 mins. and then evaporated in vacuum. The residue was dissolved in diethyl ether and washed with water and brine, dried over MgSO4 and filtered. Evaporation and flash chromatography (50% EtOAc/petroleum ether) gave (210 mg, 83%) of the product, which was used directly in the next reaction.

### Step b) 7-(Hydroxymethyl)-2-(4-hydroxyphenyl)-1,3-benzoxazol-5-ol

The title compound was prepared from 5-methoxy-7-(hydroxymethyl)-2-(4-methoxyphenyl)-1,3-benzoxazole according to the procedure of Example 20, Step e (Route b), and was obtained as a light brown solid, m.p. 282° C. (dec); MS m/e 258 (M+H)⁺.

Analysis for: C₁₄H₁₁NO₄×0.5 H₂O Calcd.: C, 63.16; H, 4.54; N, 5.26 Found: C, 63.33; H, 4.36; N, 5.04

### Example 48

### 7-(BROMOMETHYL)-2-(4-HYDROXYPHENYL)-1,3-BENZOXAZOL-5-OL

The title compound was prepared according to the procedure of Example 20, step e (Route b), from 5-methoxy-7-(hydroxymethyl)-2-(4-methoxyphenyl)-1,3-benzoxazole with prolonged stirring in the presence of boron tribromide, and was obtained as a light brown solid, m.p. 250-260° C. (dec); MS m/e 321 (M+H)⁺.

Analysis for: C₁₄H₁₀BrNO₃ Calcd: C, 52.52; H, 3.15; N, 4.38 Found: C, 52.26; H, 3.17; N, 4.07

### Example 49

### [5-HYDROXY-2-(4-HYDROXYPHENYL)-1,3-BENZOXAZOL-7-YL] ACETONITRILE

To a solution of 7-(bromomethyl)-2-(4-hydroxyphenyl)-1,3-benzoxazol-5-ol (122 mg, 0.40 mmol) in N,N-dimethylformamide (1.5 mL) was added 18-crown-6-ether (202 mg, 0.80 mmol) and potassium cyanide (131 mg, 2 mmol). The reaction mixture was allowed to stir for 2 hours and then poured into water and extracted with EtOAc. The organic extracts were washed with brine and dried over MgSO₄. Evaporation and flash chromatography (50%-60% EtOAc/petroleum ether) gave the product (80 mg, 75% yield) as a gray solid, m.p. 170-180° C.; MS m/e 265 (M−H)⁺.

Analysis for: C₁₅H₁₀N₂O₃×1.5 H₂O Calcd: C, 61.43; H, 4.47; N, 9.55 Found: C, 61.41; H, 4.21; N, 9.19

### Example 50

### 7-(1-HYDROXY-1-METHYLETHYL)-2-(4-HYDROXYPHENYL)-1,3-BENZOXAZOL-5-OL]

### Step a) 2-[5-Methoxy-2-(4-methoxyphenyl)-1,3-benzoxazole-7-yl] propan-2-ol

The title compound was prepared according to the procedure of Example 43, Step a, from 7-bromo-5-methoxy-2-(4-methoxyphenyl)-1,3-benzoxazole, employing acetone as the electrophile, to give a white solid (78% yield, m.p. 149° C.); MS m/e 314 (M+H)⁺.

Analysis for: C₁₈H₁₉NO₄ Calc'd: C, 68.99; H, 6.11; N, 4.47. Found: C, 68.78; H, 6.13; N, 4.35.

### Step b) 7-(1-Hydroxy-1-methylethyl)-2-(4-hydroxyphenyl)-1,3-benzoxazol-5-ol]

The title compound was prepared from 2-[5-methoxy-2-(4-methoxyphenyl)-1,3-benzoxazole-7-yl]propan-2-ol according to the procedure of Example 20, Step e (Route b), and was obtained as a dark brown solid (90% yield, m.p. 180-185° C.); MS m/e 286 (M+H)⁺.

Analysis for: C₁₆H₁₅NO₄×0.5 H₂O Calcd.: C, 65.30; H, 5.48; N, 4.76 Found: C, 65.03; H, 5.20; N, 4.72

### Example 51

### 2-(4-HYDROXYPHENYL)-7-ISOPROPENYL-1,3-BENZOXAZOL-5-OL

Pyridine hydrochloride (400 mg) was heated to 190° C. To the melt was added 2-[5-methoxy-2-(4-methoxyphenyl)-1,3-benzoxazole-7-yl] propan-2-ol (1 14mg, 0.36 mmol) and the reaction was stirred for 2 hours. The reaction mixture was cooled to room temperature, dissolved in water and extracted with EtOAc. The organic layers were combined and washed with HCl (1N), water then brine and dried over MgSO₄. Evaporation and purification by flash chromatography (50%-60% EtOAc/petroleum ether) gave (40 mg, 41% yield) of the product as a light red-brown solid, m.p. 225-228° C.; MS m/e 268 (M+H)⁺.

Analysis for: C₁₆H₁₃NO₃×0.5 H₂O Calcd.: C, 69.56; H, 5.11; N, 5.06 Found: C, 69.46; H, 5.22; N, 4.56

### Example 52

### 2-(4-HYDROXYPHENYL)-7-ISOPROPYL-1,3-BENZOXAZOL-5-OL]

2-(4-Hydroxyphenyl)-7-isopropenyl-1,3-benzoxazol-5-ol (64 mg, 0.24 mmol) was dissolved in a mixture of EtOAc (5 mL) and absolute ethanol (5 mL), and placed under an inert atmosphere with argon. To the solution was added 10% Pd—C (25 mg). The solution was hydrogenated on a Parr apparatus at 25 psi for 3 hours. The solution was filtered through Celitee and rinsed with ethanol. The filtrate was concentrated and the residue purified by flash chromatography (50% EtOAc/petroleum ether) to give the product (58 mg, 90% yield) as a tan solid, m.p. 200° C.; MS m/e 270 (M+H)⁺.

### Example 53

### 7-BROMO-2-(4-HYDROXY-3-(TRIFLUOROMETHYL)PHENYL)-1,3-BENZOXAZOL-5-OL

### Step a) 2-Bromo-4-methoxy-6-{[4-methoxy-3-(trifluoromethyl)benzoyl]amino}phenyl 4-methoxy-3-(trifluoromethyl)benzoate

The title compound was prepared in substantially the same manner as described in Example 20, Step c, from 2-amino-6-bromo-4-methoxyphenol and 4-methoxy-3-trifluoromethyl benzoyl chloride. The product was obtained as an off-white solid, m.p. 205-208° C.; MS m/e 622 (M+H)⁺.

Analysis for: C₂₅H₁₈BrF₆NO₆ Calc'd: C, 48.25; H, 2.92; N, 2.25 Found: C, 48.47; H, 2.76; N, 2.16

### Step b) 7-Bromo-5-methoxy-2-(4-methoxy-3-(trifluoromethyl)phenyl]-1,3-benzoxazole

The title compound was prepared in substantially the same manner as described in Example 20, Step d (Route a), from 2-bromo-4-methoxy-6-{[4-methoxy-3-(trifluoromethyl)benzoyl]amino} phenyl 4-methoxy-3-(trifluoromethyl)benzoate and p-toluenesulfonic acid monohydrate. The product was obtained as an off-white solid, m.p. 183-185° C.; MS m/e 402 (M+H)⁺.

Analysis for: C₁₆H₁₁BrF₃NO₃ Calc'd: C, 47.79; H, 2.76; N, 3.48 Found: C, 47.60; H, 2.50; N, 3.37

### Step c) 7-Bromo-2-(4-hydroxy-3-(trifluoromethyl)phenyl)-1,3-benzoxazol-5-ol

The title compound was prepared according to the procedure of Example 20, Step e (Route b), from 7-bromo-5-methoxy-2-(4-methoxy-3-(trifluoromethyl)phenyl]-1,3-benzoxazole, and was obtained as a light yellow solid (50% yield, m.p. 200-210° C.); MS m/e 372 (M−H)⁺.

Analysis for: C₁₄H₇BrF₃NO₃×0.5 H₂O Calcd: C, 43.89; H, 2.10; N, 3.65 Found: C, 43.59; H, 2.04; N, 3.6

### Example 54

### 7-(2-FURYL)-2-(4-HYDROXYPHENYL)-1,3-BENZOXAZOL-5-OL

### Step a) 7-(2-Furyl)-5-methoxy-2-(4-methoxyphenyl)-1,3-benzoxazole

7-Bromo-5-methoxy-2-(4-methoxyphenyl)-1,3-benzoxazole (300 mg, 0.90 mmol) and dichlorobis(tri-o-tolylphosphine)palladium(II) (71 mg, 0.09 mmol) were dissolved in p-xylene (3 mL) and stirred for 10 mins. at room temperature under a nitrogen atmosphere. 2-(Tributylstannyl)furan (449 mg, 1.26 mmol) was added and the reaction mixture was refluxed for 4 hours. The reaction mixture was cooled to room temperature, diluted with a saturated solution of ammonium chloride and extracted with EtOAc. The organic extracts were washed with water, then brine and dried over MgSO₄ and concentrated. Purification by flash chromatography (20%-30% EtOAc/petroleum ether) gave the title compound as a white solid (99% yield, m.p. 120-121° C.); MS m/e 322 (M+H)⁺.

Analysis for: C₁₉H₁₅NO₄ Calcd: C, 71.02; H, 4.71; N, 4.36 Found: C, 70.23; H, 4.7; N, 4.19

### Step b) 7-(2-Furyl)-2-(4-hydroxyphenyl)-1,3-benzoxazol-5-ol

The title compound was prepared according to the procedure of Example 50 and was obtained as a light pink solid (64% yield, m.p. 283-287° C.); MS m/e 294 (M+H⁺).

Analysis for: C₁₇H₁₁NO₄ Calcd: C, 69.62; H, 3.78; N, 4.78 Found: C, 69.11; H, 3.6; N, 4.64

### Example 55

### 2-(3-FLUORO-4-HYDROXYPHENYL)-7-(2-FURYL)-1,3-BENZOXAZOL-5-OL

### Step a) 2-(3-Fluoro-4-methoxyphenyl)-7-(2-furyl)-5-methoxy-1,3-benzoxazole

The title compound was prepared according to the procedure of Example 53, Step a, from 7-bromo-5-methoxy-2-(4-methoxy-3-(trifluoromethyl)phenyl]-1,3-benzoxazole, and was obtained as amber crystals (73% yiled, m.p. 155° C.); MS m/e 340 (M+H)⁺.

Analysis for: C₁₉H₁₄FNO₄ Calcd: C, 67.25; H, 4.16; N, 4.13 Found: C, 66.88; H, 3.97; N, 4.04

### Step b) 2-(3-Fluoro-4-hydroxyphenyl)-7-(2-furyl)-1,3-benzoxazol-5-ol

The title compound was prepared according to the procedure of Example 50, from 2-(3-fluoro-4-methoxyphenyl)-7-(2-furyl)-5-methoxy-1,3-benzoxazole, and was obtained as a gray solid (81% yield, m.p. 245-250° C.); MS m/e 312 (M+H)⁺.

Analysis for: C₁₇H₁₀FNO₄×0.7 C₃H₆O Calcd: C, 65.04; H, 4.37; N, 3.79 Found: C, 64.84; H, 4.29; N, 3.70

### Example 56

### 2-(4-HYDROXYPHENYL)-7-THIEN-2-YL-1,3-BENZOXAZOL-5-OL

### Step a) 5-Methoxy-2-(4-methoxyphenyl)-7-thien-2-yl)-1,3-benzoxazole

The title compound was prepared according to the procedure of Example 53, Step a, from 7-bromo-5-methoxy-2-(4-methoxyphenyl)-1,3-benzoxazole and 2-(tributylstannyl)thiophene. The product was obtained as a white solid (95% yield), m.p. 95-100° C.); MS m/e 338 (M+H).

### Step b) 2-(4-Hydroxyphenyl)-7-thien-2-yl-1,3-benzoxazol-5-ol

The title compound was prepared according to the procedure of Example 50, from 5-methoxy-2-(4-methoxyphenyl)-7-thien-2-yl)-1,3-benzoxazole and was obtained as a gray solid (80% yield, m.p. 278-280° C.); MS m/e 310 (M+H)⁺.

Analysis for: C₁₇H₁₁NO₃S×0.25 H₂O Calcd: C, 65.06; H, 3.69; N, 4.46 Found: C, 64.93; H, 3.84; N, 4.21

### Example 57

### 2-(4-HYDROXYPHENYL)-7-(1,3-TH IAZOL-2-YL)-1,3-BENZOXAZOL-5-OL

### Step a) 5-methoxy-2-(4-methoxyphenyl)-7-(1,3-thiazol-2-yI)-1,3-benzoxazole

The title compound was prepared according to the procedure of Example 53, Step a, from 7-bromo-5-methoxy-2-(4-methoxyphenyl)-1,3-benzoxazole and 2-(tributylstannyl)thiazole. The product was obtained as an off white solid (93% yield, m.p. 132-136° C.); MS m/e 339 (M+H)⁺.

Analysis for: C₁₈H₁₄N₂O₃S Calcd: C, 63.89; H, 4.17; N, 8.28 Found: C, 63.53; H, 3.94; N, 8.15

### Step b) 2-(4-Hydroxyphenyl)-7-(1,3-thiazol-2-yl)-1,3-benzoxazol-5-ol

The title compound was prepared according to the procedure of Example 50, from 5-methoxy-2-(4-methoxyphenyl)-7-(1,3-thiazol-2-yl)-1,3-benzoxazole, and was obtained as a yellow solid (55% yield, m.p. 245-255° C.); MS m/e 311 (M+H)⁺.

Analysis for: C₁₆H₁₀N₂O₃S×1.5 H₂O Calcd: C, 56.97; H, 3.88; N, 8.30 Found: C, 57.24; H, 3.95; N, 7.50

### Example 58

### 2-(3-FLUORO-4-HYDROXYPHENYL)-5-HYDROXY-1,3-BENZOXAZOLE-7-CARBONITRILE

The title compound was prepared according to the procedure of Example 35, from 7-bromo-2-(3-fluoro-4-methoxyphenyl)-5-methoxy-1,3-benzoxazole and zinc cyanide. The product was obtained as a white solid, m.p. 308-310° C., MS m/e 269 (M−H)⁺.

Analysis for: C₁₄H₇FN₂O3×1.5 H₂O Calcd: C, 61.01; H, 2.77; N, 10.16 Found: C, 60.68; H, 2.46; N, 9.77

### Examples 59 and 60

### 4-BROMO-2-(4-HYDROXYPHENYL)-7-METHOXY-1,3-BENZOXAZOL-5-OL (EX. 59)

### 4,6-DIBROMO-2-(4-HYDROXYPHENYL)-7-METHOXY-1,3-BENZOXAZOL-5-OL (EX. 60)

The title compounds were prepared according to the procedure of Example 28, from 2-(4-hydroxyphenyl)-7-methoxy-1,3-benzoxazol-5-ol and N-bromosuccinimide. Product (Ex. 59) was obtained as a white solid, m.p. 246-248° C., MS m/e 336 (M+H)⁺.

Analysis for: C₁₄H₁₀BrNO₄×1 H₂O Calcd: C, 49.49; H, 3.08; N, 4.12 Found: C, 49.28; H, 2.89; N, 3.87.

Product (Ex. 60) was obtained as a white solid, m.p. 260-262° C., MS m/e 414 (M+H)⁺.

Analysis for C₁₄H₉Br₂NO₄ Calcd: C, 40.52; H, 2.19; N, 3.37 Found: C, 40.21; H, 2.00; N, 3.3

### Example 61

### 7-BROMO-2-(3,5-DIFLUORO-4-HYDROXYPHENYL)-1,3-BENZOXAZOL-5-OL

The title compound was prepared in substantially the same manner as described in Example 21, from 3,5-difluoro-4-methoxybenzoic acid, and 2-amino-6-bromo-4-methoxyphenol, and was obtained as a white solid, m.p. 270-272° C.; MS m/e 340 (M−H)⁺.

Analysis for: C₁₃H₆BrF₂NO₃ Calc'd: C, 45.64; H, 1.77; N, 4.09 Found: C, 45.81; H, 1.73; N, 3.89

### Example 62

### 2-(3, 5-DIFLUORO-4-HYDROXYPHENYL)-7-VINYL-1,3-BENZOXAZOL-5-OL

The title compound was prepared in substantially the same manner as described in Example 24, Route b, from 7-bromo-2-(3,5-difluoro-4-hydroxyphenyl)-1,3-benzoxazol-5-ol, and was obtained as a white solid, m.p. 160-262° C.; MS m/e 288 (M−H)⁺.

Analysis for: C₁₅H₉F₂NO_{3×0.1} H₂O Calc'd: C, 61.52; H, 3.23; N, 4.78 Found: C, 61.53; H, 3.10; N, 4.72

### Example 63

### 7-BROMO-2-(4-HYDROXY-2-METHYLPHENYL)-1,3-BENZOXAZOL-5-OL

The title compound was prepared in substantially the same manner as described in Example 21, from 4-methoxy-2-methylbenzoic acid, and 2-amino-6-bromo-4-methoxyphenol, and was obtained as a light purple solid, m.p. 120-135° C.; MS m/e 320 (M+H)⁺.

Analysis for: C₁₄H₁₀BrNO₃ Calc'd: C, 52.52; H, 3.15; N, 4.38 Found: C, 52.24; H, 2.97; N, 4.15

### Example 64

### 2-(3-FLUORO-4-HYDROXYPHENYL)-7-(1-FLUOROVI NYL)-1,3-BENZOXAZOL-5-OL

Hydrogen fluoride pyridine (1.14 mL) was added dropwise into a cold (0° C.) solution of 2-[4-(acetyloxy)-3-fluorophenyl]-7-vinyl-1,3-benzoxazol-5-yl acetate (0.25 g, 0.7 mmol), in sulfolane (3 mL). The reaction mixture was stirred for 5 mins. and then 1,3-dibromo-5,5-dimethylimidazolidine-2,4-dione (120 mg) was added in one portion. The reaction mixture was stirred at room temperature for 24 hours, diluted with HCl (1N) and extracted with EtOAc. The organic layer was dried over MgSO₄. Evaporation and purification by flash chromatography (CH₂Cl₂/isopropyl alcohol 0.3%) gave 7-(2-bromo-1-fluoroethyl)-2-(3-fluoro-4-hydroxyphenyl)-1,3-benzoxazol-5-ol as a white solid (0.25 9, m.p. 185-186° C.). The product was taken into acetonitrile (2 mL) and 1,8-diazabicyclo[5.4.0]undec-7-ene (150 mg) was added. The reaction mixture was stirred for 24 hours, poured into cold (0° C.) HCl (1N, 10 mL) and extracted with EtOAc. The organic extracts were dried over MgSO₄. Evaporation and purification by flash chromatography (20% EtOAc/hexanes) gave a white solid (160 mg, m.p. 213-214° C.); MS m/e 290 (M+H)⁺.

Analysis for: C₁₅H₉BrF₂NO3×0.3 H₂O Calc'd: C, 61.15; H, 3.28; N, 4.75 Found: C, 60.84; H, 3.41; N, 4.57

### Example 65

### PREPARATION AND ANALYSIS OF METABOLITES OF 2-(3′-FLUORO-4′-HYDROXYPHENYL)-7-VINYL-1,3-BENZOXAZOL-5-OL (ERB-041)

Large-scale incubations of ERB-041 in rat liver microsomes and cytosol were carried out to isolate the glucuronides (designated herein M5 and M6) and sulfates (designated herein M9 and M9A) for NMR analysis. Based on the results from ¹H- and ¹⁹F-NMR analyses, the structures of M5 and M6 were unambiguously assigned as ERB-041-4′-glucuronide (M5) and ERB-041-5-glucuronide (M6). The structures of M9 and M9A were determined to be ERB-041-4′-sulfate and ERB-041-5-sulfate, respectively.

The structures of M5, M6, M9 and M9A are shown below:

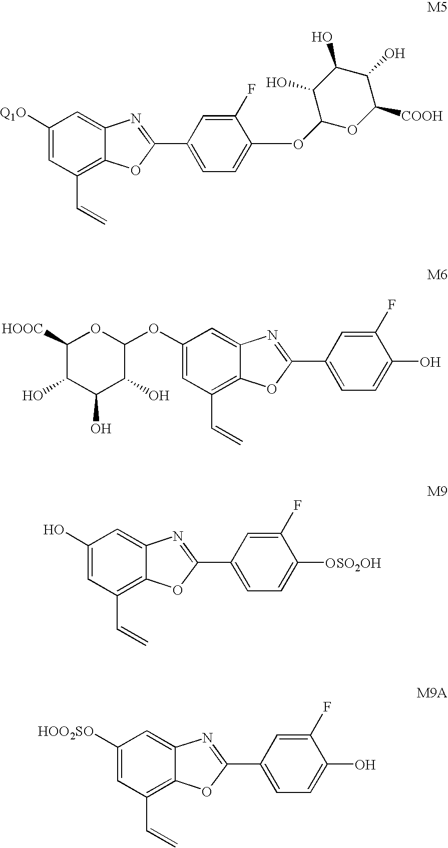

Liver microsomes (male, lot VJF, 20 mg/mL) and cytosol (male, lot 100007, 20 mg/mL) from SD rats were obtained from In Vitro Technologies, Inc., Baltimore, Md. Additional rat liver incubations were conducted using cytosol obtained from BD Gentest, Woburn, Mass. The co-factors uridine 5′-diphosphoglucuronic acid (UDPGA) and 3′-phosphoadenosine-5′-phosphosulfate (PAPS) were purchased from Sigma Chemical Co, St Louis, Mo. All other reagents were of analytical grade.

Microsomal Incubations of ERB-041 in the Presence of UDPGA

An analytical scale incubation of ERB-041 with male SD rat liver microsomes in the presence of UDPGA was conducted in phosphate buffer (0.1 M, pH 7.4) containing 1 mg/mL rat liver microsomal protein with final concentrations of 5 mM magnesium chloride and 4 mM UDPGA. This pilot incubation (1.0 mL incubation volume) was conducted using a substrate concentration of 100 μM at 37° C. for 60 mins. Incubations were terminated by the addition of an equal volume of chilled acetonitrile.

Large-scale incubations (a total of 37 incubations @ 5.0 mL incubation volume) to generate sufficient quantities of the glucuronide metabolites for structure elucidation were subsequently conducted as described above at 37° C. for 60 mins. Appropriate substrate controls and incubations without addition of UDPGA were also carried out.

Cytosolic Incubations of ERB-041 in the Presence of PAPS

Analytical scale incubations of ERB-041 with male SD rat and human liver cytosolic fractions in the presence of PAPS were conducted in tris buffer (50 mM, pH 7.4) containing 1 mg/mL liver cytosol, 0.114 mg/mL PAPS, 0.1 mg/mL BSA, 5 mM dithiothreitol, and 5 mM MgCl₂. These pilot incubations (1.0 mL incubation volume) were conducted using a substrate concentration of 100 μM ERB-041 at 37° C. for 60 mins. Incubations were terminated by the addition of an equal volume of chilled acetonitrile.

Large-scale incubations (a total of 60 incubations @ 1.0 mL incubation volume) to generate sufficient quantities of the sulfate metabolites for structure elucidation were subsequently conducted as described above at 37° C. for 60 mins. Appropriate substrate controls and incubations without addition of PAPS were conducted. Additional incubations (a total of 120 @ 2.0 mL incubation volume, 50 μM ERB-041) were also conducted in a similar manner to isolate sufficient amounts of ERB-041-sulfates to enable full structural identification.

Sample Preparation

Following termination of the incubations, the reaction mixtures were centrifuged (3000 rpm, 10-15 min) and the supernatants were then used in subsequent preparative HPLC isolations.

High Performance Liquid Chromatography

Reserved phase-HPLC was utilized for all metabolite analysis. To confirm the formation of glucuronide and sulfate conjugates of ERB-041 in pilot scale incubations, HPLC analysis was performed using an Agilent 1100 LC system equipped with a diode array detector (Agilent Technologies, Wilmington, Del.). The diode array detector was set at a wavelength of 254 nm. Separations were achieved using a Phenomenex Prodigy, 5 ODS [4.6×250 mm] column (Phenomenex, Inc. Torrance, Calif.) and 1.0 mumin flow rate using gradient system A.

Gradient (A)

| | A % | |
|---|---|---|
| Time | (10 mM Ammonium | B % |
| (min) | Acetate, pH 4.5) | (Acetonitrile) |
| 0 | 95 | 5 |
| 5 | 95 | 5 |
| 28 | 35 | 65 |
| 33 | 5 | 95 |
| 42 | 5 | 95 |
| 43 | 95 | 5 |
| 48 | 95 | 5 |

Analytical detection during the large-scale analysis of incubation extracts was achieved under the following conditions:

A Waters 2690 Alliance LC system with UV detection (254 and 280 nm) (Waters Corp., Milford, Mass.) was used and separations were achieved using a Phenomenox LUNA®-phenyl/hexyl column [4.6×250 mm, 5 μ] using gradient system B (glucuronides) and gradient system C (sulfates).

Gradient (B)

| | A % (5 mM | | |
|---|---|---|---|
| Time | Ammonium | B % | Flow |
| (min) | Acetate, pH 4.5) | (Acetonitrile) | (mL/min) |
| 0 | 92 | 8 | 0.8 |
| 2 | 92 | 8 | 0.8 |
| 35 | 55 | 45 | 0.8 |
| 37 | 10 | 90 | 1.0 |
| 45 | 10 | 90 | 1.0 |
| 47 | 92 | 8 | 1.0 |

Gradient (C)

| | A % (5 mM | | |
|---|---|---|---|
| Time | Ammonium | B % | Flow |
| (min) | Acetate, pH 4.5) | (Acetonitrile) | (mL/min) |
| 0 | 85 | 15 | 0.8 |
| 2 | 85 | 15 | 0.8 |
| 35 | 55 | 45 | 0.8 |
| 37 | 10 | 90 | 1.0 |
| 45 | 10 | 90 | 1.0 |
| 47 | 85 | 15 | 0.8 |

Preparative HPLC isolation of the conjugated metabolites was achieved under the following conditions:

A Waters Delta Prep 4000 system with UV detection (254 and 280 nm) was used and separations were achieved using a Zorbaxe RX-C18 column [21.1×250 mm, 10 μ] (Agilent Technologies, Wilmington, Del.) column using gradient system D (gluconides) and E (sulfates).

Gradient (D)

| | A % (5 mM | | |
|---|---|---|---|
| Time | Ammonium | B % | Flow |
| (min) | Acetate, pH 4.5) | (Acetonitrile) | (mL/min) |
| 0 | 95 | 5 | 20 |
| 2 | 95 | 5 | 20 |
| 60 | 60 | 40 | 20 |
| 62 | 20 | 80 | 22 |
| 70 | 20 | 80 | 22 |
| 72 | 95 | 5 | 20 |

Gradient (E)

| | A % (5 mM | | |
|---|---|---|---|
| Time | Ammonium | B % | Flow |
| (min) | Acetate, pH 4.5) | (Acetonitrile) | (mL/min) |
| 0 | 90 | 10 | 20 |
| 2 | 90 | 10 | 20 |
| 60 | 55 | 45 | 20 |
| 62 | 40 | 60 | 22 |
| 70 | 20 | 80 | 22 |
| 72 | 90 | 10 | 20 |

Isolation and Purification of Conjugated Metabolites

For glucuronides, the combined extracts from rat liver microsomal incubations in the presence of UDPGA were subjected to reversed phase flash chromatography with sequential elution with water and methanol. The fractions containing ERB-041 isomeric glucuronides (M5 and M6) were combined and concentrated prior to further isolation by preparative HPLC. Preparative HPLC isolation of the conjugated metabolites was conducted using a Waters Delta Prep 4000 system on a Zorbax® RX-C18 column [21.1×250 mm, 10 μ] using gradient D. The separation was monitored by UV detection at 254 and 280 nm and the peaks containing the metabolites of interest (Rt=26.8-27.3 min, M5 and Rt=28.0-28.5 min, M6) were collected. After the organic solvent was evaporated under vacuum, the residues were lyophilized to get pure glucuronides (M5 and M6) for subsequent 1 H- and 19F-NMR analysis.

For sulfates, the combined crude incubation products were isolated by preparative HPLC on a Zorbaxe RX C-18 column. Preparative HPLC isolation was achieved using gradient solvent E with UV monitoring at 280nm and 250 nm. The peaks of interest, M9A (Rt=28.5-29 min) and M9, (Rt=29.3-29.8 min) were collected. After the organic solvents were evaporated under vacuum, the residues were lyophilized to get pure sulfate conjugates (M9A and M9) for subsequent 19F-NMR analysis.

Analytical detection during the preparative HPLC and analysis of purified glucuronides and sulfates was done as described supra using gradient B and C for glucuronides and sulfates, respectively.

LC-MS Analysis

LC-MS characterization of the isolated metabolites was performed using an Agilent 1100 HPLC system coupled with HP 1100 MSD mass spectrometer. Full scan Electro-Spray Ionization (ESI) mass spectra were acquired at unit resolution. ESI positive ionization mode was utilized for glucuronides' mass spectral recording, whereas ESI negative mode was selected for sulfates' mass spectral recording. An XTerra® C18 column (2.1×250 mm, 5 μ; Waters Corp.) was used with gradient F (below) as the solvent system.

Gradient (F)

| | A % (5 mM | | |
|---|---|---|---|
| Time | Ammonium | B % | Flow |
| (min) | Acetate, pH 4.5) | (Acetonitrile) | (mL/min) |
| 0 | 82 | 18 | 0.22 |
| 2 | 82 | 18 | 0.22 |
| 35 | 50 | 50 | 0.22 |
| 37 | 10 | 90 | 0.22 |
| 45 | 10 | 90 | 0.22 |
| 47 | 82 | 18 | 0.22 |

HPLC conditions used for the LC-MS analysis to confirm the formation of glucuronide conjugates was previously described. Large-scale generation of the sulfate metabolites was confirmed using the same LC-MS conditions as described above (Gradient A) except that a 2×250 mm column and 0.35 mL/min flow were used. The HPLC system used was a Waters Alliance 2690 HPLC pumping system. The mass spectrometer used was a Finnigan TSQ Quantum (Thermo Finnigan, San Jose, Calif.) equipped with an electrospray ionization (ESI) source and operated in the negative ionization mode. Unit mass resolution was used for all analyses.
NMR Analysis

In order to compare the ¹H-NMR spectra of glucuronides with that of ERB-041, the chemical shifts of ERB-041 were unambiguously assigned based on 1H-NMR, ¹³C-NMR, HMBC, and HMQC experiments. All NMR analyses were acquired using a Bruker 400 AMX spectrometer (Bruker, Billerica, Mass.).

For glucuronides, CD₃CN/DMSO-d6 mixture was used as the solvent for ¹H-NMR and CD₃OD was used for ¹⁹F-NMR analyses. For sulfate conjugates, CD30D containing 0.005% TFA was used for ¹⁹F-NMR analysis and fluoro-benzene standard (δF -113.12 ppm) was used to adjust instrument settings prior to data acquisition of sulfate conjugates.

Data Analysis

Agilent chromatography software, ChemStation for LC 3D, version Rev. A. 09. 01 (Agilent Technologies Inc., Wilmington, Del.) was utilized for detection of the metabolite peaks. Xcalibur version 1.3 software was used for control of LC-MS equipment and recording of data from LC-MS analyses.

Glucuronidation in Rat Liver Microsomes

When incubation of ERB-041 with microsomal proteins from rat liver microsomes was conducted in the presence of UDPGA, two major metabolites, M5 and M6, were detected. LC-MS analysis (ESI, positive ionization) of M5 and M6 indicated that these peaks are phenolic glucuronides of ERB-041 at the 4′-OH (phenyl) and 5-OH (benzoxazole) positions. Further confirmation of the formation of M5 and M6 in the large-scale incubations was demonstrated through LC-MS analysis (ESI, positive ionization). Separation of the two metabolites was achieved using gradient solvent system B as described supra.

Sulfation in Rat Liver Cytosol

When incubations of ERB-041 with cytosol from rat and human liver fractions were conducted in the presence of PAPS, two metabolites (M9A and M9) were detected by HPLC and LC-MS analysis. Rat liver and human liver cytosol incubations produced similar profiles. Both metabolites M9A and M9 yielded [M−H]- at m/z 350 consistent with ERB-041-sulfate in both human and rat cytosolic incubations. Further confirmation of the identity of the isolated metabolites M9A and M9 from the large-scale incubations was obtained using LC-MS analysis (ESI, negative ionization). In the large-scale incubation, approximately 23% of ERB-041 was converted to the two sulfate conjugates. Separation of the two sulfate metabolites was achieved using gradient solvent system C described supra.

Structure Elucidation of ERB-041 Conjugated Metabolites

Mass spectra were obtained for ERB-041 glucuronides (metabolites M5 and M6) and sulfates (metabolites M9 and M9A) and confirmed their presence in the large-scale incubation extracts. LC-MS data indicated phenolic glucuronidation and/or sulfation of both rings (phenyl and benzoxazole). The sites of conjugation were determined based on ¹⁹F- and ¹H-NMR analysis. Detailed mass spectral and NMR data of the individual metabolites are discussed below.

Metabolite M5 (ERB-041-4′-Glucuronide)

Metabolite M5 exhibited a [M+H]⁺ at m/z 448; therefore, metabolite M5 was confirmed to be an ERB-041-glucuronide at either of the phenolic groups of the phenyl (C-4′) or benzoxazole (C-5) rings as previously reported. This was further supported by the presence of [M+ Na]⁺ at m/z 470 (+22 mass units, Na adduct) and m/z 272 (loss of glucuronide moiety). The lack of any diagnostic mass spectral fragments, however, did not allow for distinguishing individual sites of glucuronidation based on the LC-MS data.

Further elucidation of the structure was obtained from ¹⁹F- and ¹H-NMR data. ¹⁹F-NMR analysis of ERB-041 exhibited a signal (3′-F) with a chemical shift of δF-138 ppm. Results from ¹⁹F-NMR analysis of metabolites M5 and M6 indicated that the 3′-F signal in M5 shifted to 8F-134 ppm, whereas that of M6 remained unaffected at 8F-138 ppm. These results are clearly consistent with the site of glucuronidation being the C4′-position (phenyl ring) for M5. Glucuronidation at the C4′-OH position was further confirmed through ¹H-NMR analysis. The proton NMR spectrum of this metabolite was similar to that of ERB-041 except for the significant downfield shift of H-5′ (from δ 7.19 ppm in ERB-041 to δ 7.45 ppm in M5). In addition, a signal at δ 5.2 ppm consistent with an anomeric proton was also evident in the metabolite spectrum, further indicative of a glucuronide structure. Chemical shifts of all other protons remained unchanged. That the H-5′ signal was the only signal that underwent the down field shift with all protons of the benzoxazole ring remaining unaffected, further confirms the site of glucuronidation to be 4′-phenolic group of the phenyl ring. It is noted that a duplicate set of signals was evident in the 1H-NMR spectrum of M5 consistent with P and a epimers of glucuronic acid. The observed NOEs between the anomeric proton of the glucuronic acid moiety with H-5′ in the ROSEY spectrum of M5 was in agreement and further supports the assigned structure as 4′-O-glucuronide.

Metabolite M6 (ERB-041-5-Glucuronide)

Similar to metabolite M5, the mass spectral analysis of M6 exhibited a [M+H]⁻ at m/z 448, confirming its identity as an ERB-041 monoglucuronide. Further support of the identity was through the presence of mlz 470 ([M+ Na]⁺) and m/z 272 (loss of glucuronide moiety), as discussed above. Again, because of the lack of any diagnostic mass spectral fragments, the individual sites of glucuronidation could not be distinguished based solely on the LC-MS data. As discussed above, ¹⁹F-NMR analysis of M6 indicated that the 3′-F signal in M6 remained unaffected by glucuronidation and displayed a chemical shift of δF -138 ppm, the same as that of parent ERB-041 (δF -138 ppm). These results are clearly consistent with the site of glucuronidation being the C5-OH position (benzoxazole ring) for M6. Glucuronidation at C-5 was further confirmed through 1H-NMR analysis, where the proton signals corresponding to H-4 and H-6 clearly underwent significant downfield shifts compared to parent ERB-041. The ¹H-NMR results were consistent and further confirmed the site of glucuronidation to be the C5-phenolic group of the benzoxazole ring.

Metabolite M9 (ERB-041-4′-Sulfate)

Metabolite M9 exhibited a molecular ion [M−H]⁻ at m/z 350 with a fragment at m/z 270 due to loss of sulfate moiety; therefore, M9 was confirmed to be ERB-041-sulfate. Sulfation could have taken place at either of the phenolic groups (C-4′, phenyl or C-5, benzoxazole rings). The lack of any diagnostic mass spectral fragments, however, did not allow for distinguishing individual sites of sulfation based on the LC-MS data. Unambiguous structural assignment of the sulfate metabolite was made based on additional ¹⁹F-NMR analysis. Thus, ¹⁹F-NMR analysis of M9 exhibited a signal (3′-F) with a chemical shift of 5F -130 ppm compared to δF -138 ppm for that of ERB-041. As discussed for glucuronides M5 and M6 above, the significant downfield shift observed here is clearly indicative of sulfate conjugation at the C-4′ position. This assignment was further confirmed when the chemical shift observed for 3′-F in M9A remained unaffected at 6F -138 ppm.

Metabolite M9A (ERB-041-5-Sulfate)

Metabolite M9A exhibited the same molecular ion [M−H]⁻ at m/z 350 with a fragment at m/z 270 corresponding to the loss of sulfate as seen with M9; therefore, metabolite M9A was also concluded to be a direct ERB-041-sulfate conjugate. Like that of M9, either C4′ (phenyl) or C5 (benzoxazole) are available sites for sulfation. Again, the lack of any diagnostic mass spectral fragments did not allow for distinguishing individual sites of sulfation based on the LC-MS data. Unambiguous structural assignment of M9A was made based on ¹⁹F-NMR analysis. No change in the chemical shift was observed for the 8F of M9A compared to ERB-041. In both cases, the 5F observed was −138 ppm, a result consistent with sulfation at the distant C5-benzoxazole OH group.

It is intended that each of the patents, applications, and printed publications, including books, mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A compound of formula I, having the structure: 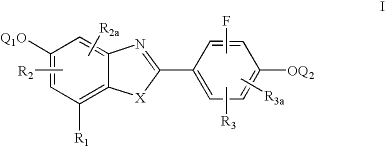
wherein:
Q₁ and Q₂ are independently H, a modified or unmodified hexose residue or S(O)ₜ—OH, provided that Q₁ and Q₂ are not both H;
t is 0, 1 or 2;
R₁ is alkenyl of 2-7 carbon atoms; wherein the alkenyl moiety is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt thereof.

2. The compound of claim 1 wherein t is 2.

3. The compound according to claim 2, wherein X is O; and R₁ is alkenyl of 2-3 carbon atoms, which is optionally substituted with hydroxyl, —CN, halogen, trifluroalkyl, trifluoroalkoxy, —COR₅, —CO₂R₅, —NO₂, CONR₅R₆, NR₅R₆ or N(R₅)COR₆.

4. The compound of claim 3 wherein Q₁ and Q₂ are independently selected from —S(O)₂—OH and a modified or unmodified hexose residue.

5. The compound of claim 4 wherein the modified hexose residue is a glucuronide residue.

6. The compound of claim 3 wherein Q₁ and Q₂ are independently selected from —S(O)₂—OH and H.

7. The compound of claim 3 wherein Q₁ and Q₂ are independently selected from H and a modified or unmodified hexose residue.

8. The compound of claim 7 wherein the modified hexose residue is a glucuronide residue.

9. The compound of claim 8 wherein the modified hexose residue is a glucuronide residue.

10. The compound according to claim 1, which is 2-(3′-fluoro-4′-glucuronide phenyl)-7-vinyl-1,3-benzoxazol-5-ol or a pharmaceutically acceptable salt thereof.

11. The compound according to claim 1, which is 2-(3′-fluoro-4′-sulfate phenyl)-7-vinyl-1,3-benzoxazol-5-ol or a pharmaceutically acceptable salt thereof.

12. The compound according to claim 1, which is 2-(3′-fluoro-4′-hydroxy phenyl)-7-vinyl-1,3-benzoxazol-5-glucuronide or a pharmaceutically acceptable salt thereof.

13. The compound according to claim 1, which is 2-(3′-fluoro-4′-hydroxy phenyl)-7-vinyl-1,3-benzoxazol-5-sulfate or a pharmaceutically acceptable salt thereof.

14. The compound according to claim 1, which is 2-(3′-fluoro-4′-glucuroride phenyl)-7-vinyl-1,3-benzoxazol-5-glucuronide or a pharmaceutically acceptable salt thereof.

15. The compound according to claim 1, which is 2-(3′-fluoro-4′-glucuronide phenyl)-7-vinyl-1,3-benzoxazol-5-sulfate or a pharmaceutically acceptable salt thereof.

16. The compound according to claim 1, which is 2-(3′-fluoro-4′-sulfate phenyl)-7-vinyl-1,3-benzoxazol-5-glucuronide or a pharmaceutically acceptable salt thereof.

17. The compound according to claim 1, which is 2-(3′-fluoro-4′-sulfate phenyl)-7-vinyl-1,3-benzoxazol-5-sulfate or a pharmaceutically acceptable salt thereof.

18. The compound according to claim 1, which is 2-(2′-fluoro-4′-glucuronide phenyl)-7-vinyl-1,3-benzoxazol-5-ol or a pharmaceutically acceptable salt thereof.

19. The compound according to claim 1, which is 2-(2′-fluoro-4′-sulfate phenyl)-7-vinyl-1,3-benzoxazol-5-ol or a pharmaceutically acceptable salt thereof.

20. The compound according to claim 1, which is 2-(2′-fluoro-4′-hydroxy phenyl)-7-vinyl-1,3-benzoxazol-5-glucuronide or a pharmaceutically acceptable salt thereof.

21. The compound according to claim 1, which is 2-(2′-fluoro-4′-hydroxy phenyl)-7-vinyl-1,3-benzoxazol-5-sulfate or a pharmaceutically acceptable salt thereof.

22. The compound according to claim 1, which is 2-(2′-fluoro-4′-glucuronide phenyl)-7-vinyl-1,3-benzoxazol-5-glucuronide or a pharmaceutically acceptable salt thereof.

23. The compound according to claim 1, which is 2-(2′-fluoro-4′-glucuronide phenyl)-7-vinyl-1,3-benzoxazol-5-sulfate or a pharmaceutically acceptable salt thereof.

24. The compound according to claim 1, which is 2-(2′-fluoro-4′-sulfate phenyl)-7-vinyl-1,3-benzoxazol-5-glucuronide or a pharmaceutically acceptable salt thereof.

25. The compound according to claim 1, which is 2-(2′-fluoro-4′-sulfate phenyl)-7-vinyl-1,3-benzoxazol-5-sulfate or a pharmaceutically acceptable salt thereof.

26. The compound according to claim 1, which is 2-(2′,3′-difluoro-4′-glucuronide phenyl)-7-vinyl-1,3-benzoxazol-5-ol or a pharmaceutically acceptable salt thereof.

27. The compound according to claim 1, which is 2-(2′,3′-difluoro-4′-sulfate phenyl)-7-vinyl-1,3-benzoxazol-5-ol or a pharmaceutically acceptable salt thereof.

28. The compound according to claim 1, which is 2-(2′,3′-difluoro-4′-hydroxyphenyl)-7-vinyl-1,3-benzoxazol-5-glucuronide or a pharmaceutically acceptable salt thereof.

29. The compound according to claim 1, which is 2-(2′,3′-difluoro-4′-hydroxyphenyl)-7-vinyl-1,3-benzoxazol-5-sulfate or a pharmaceutically acceptable salt thereof.

30. The compound according to claim 1, which is 2-(2′,3′-difluoro-4′-glucuronide phenyl)-7-vinyl-1,3-benzoxazol-5-glucuronide or a pharmaceutically acceptable salt thereof.

31. The compound according to claim 1, which is 2-(2′,3′-difluoro-4′-glucuronide phenyl)-7-vinyl-1,3-benzoxazol-5-sulfate or a pharmaceutically acceptable salt thereof.

32. The compound according to claim 1, which is 2-(2′,3′-difluoro-4′-sulfate phenyl)-7-vinyl-1,3-benzoxazol-5-glucuronide or a pharmaceutically acceptable salt thereof.

33. The compound according to claim 1, which is 2-(2′,3′-difluoro-4′-sulfate phenyl)-7-vinyl-1,3-benzoxazol-5-sulfate or a pharmaceutically acceptable salt thereof.

34. The compound according to claim 1, which is 4-bromo-2-(3′-fluoro-4′-glucuronide phenyl)-7-vinyl-1,3-benzoxazol-5-ol or a pharmaceutically acceptable salt thereof.

35. The compound according to claim 1, which is 4-bromo-2-(3′-fluoro-4′-sulfate phenyl)-7-vinyl-1,3-benzoxazol-5-ol or a pharmaceutically acceptable salt thereof.

36. The compound according to claim 1, which is 4-bromo-2-(3′-fluoro-4′-hydroxyphenyl)-7-vinyl-1,3-benzoxazol-5-glucoronide or a pharmaceutically acceptable salt thereof.

37. The compound according to claim 1, which is 4-bromo-2-(3′-fluoro-4′-hydroxyphenyl)-7-vinyl-1,3-benzoxazol-5-sulfate or a pharmaceutically acceptable salt thereof.

38. The compound according to claim 1, which is 4-bromo-2-(3′-fluoro-4′-glucuronide phenyl)-7-vinyl-1,3-benzoxazol-5-glucuronide or a pharmaceutically acceptable salt thereof.

39. The compound according to claim 1, which is 4-bromo-2-(3′-fluoro-4′-glucuronide phenyl)-7-vinyl-1,3-benzoxazol-5-sulfate or a pharmaceutically acceptable salt thereof.

40. The compound according to claim 1, which is 4-bromo-2-(3′-fluoro-4′-sulfate phenyl)-7-vinyl-1,3-benzoxazol-5-glucuronide or a pharmaceutically acceptable salt thereof.

41. The compound according to claim 1, which is 4-bromo-2-(3′-fluoro-4′-sulfate phenyl)-7-vinyl-1,3-benzoxazol-5-sulfate or a pharmaceutically acceptable salt thereof.

42. The compound according to claim 1, which is 4,6-dibromo-2-(3′-fluoro-4′-glucuronide phenyl)-7-vinyl-1,3-benzoxazol-5-ol or a pharmaceutically acceptable salt thereof.

43. The compound according to claim 1, which is 4,6-dibromo-2-(3′-fluoro-4′-sulfate phenyl)-7-vinyl-1,3-benzoxazol-5-ol or a pharmaceutically acceptable salt thereof.

44. The compound according to claim 1, which is 4,6-dibromo-2-(3′-fluoro-4′-hydroxyphenyl)-7-vinyl-1,3-benzoxazol-5-glucuronide or a pharmaceutically acceptable salt thereof.

45. The compound according to claim 1, which is 4,6-dibromo-2-(3′-fluoro-4′-hydroxyphenyl)-7-vinyl-1,3-benzoxazol-5-sulfate or a pharmaceutically acceptable salt thereof.

46. The compound according to claim 1, which is 4,6-dibromo-2-(3′-fluoro-4′-glucuronide phenyl)-7-vinyl-1,3-benzoxazol-5-glucuronide or a pharmaceutically acceptable salt thereof.

47. The compound according to claim 1, which is 4,6-dibromo-2-(3′-fluoro-4′-glucuronide phenyl)-7-vinyl-1,3-benzoxazol-5-sulfate or a pharmaceutically acceptable salt thereof.

48. The compound according to claim 1, which is 4,6-dibromo-2-(3′-fluoro-4′-sulfate phenyl)-7-vinyl-1,3-benzoxazol-5-glucuronide or a pharmaceutically acceptable salt thereof.

49. The compound according to claim 1, which is 4,6-dibromo-2-(3′-fluoro-4′-sulfate phenyl)-7-vinyl-1,3-benzoxazol-5-sulfate or a pharmaceutically acceptable salt thereof.

50. The compound according to claim 1, which is 7-(1-bromovinyl)-2-(2′-fluoro-4′-glucuronide phenyl)-1,3-benzoxazol-5-ol or a pharmaceutically acceptable salt thereof.

51. The compound according to claim 1, which is 7-(1-bromovinyl)-2-(2′-fluoro-4′-sulfate phenyl)-1,3-benzoxazol-5-ol or a pharmaceutically acceptable salt thereof.

52. The compound according to claim 1, which is 7-(1-bromovinyl)-2-(2′-fluoro-4′-hydroxyphenyl)-1,3-benzoxazol-5-glucuronide or a pharmaceutically acceptable salt thereof.

53. The compound according to claim 1, which is 7-(1-bromovinyl)-2-(2′-fluoro-4′-hydroxyphenyl)-1,3-benzoxazol-5-sulfate or a pharmaceutically acceptable salt thereof.

54. The compound according to claim 1, which is 7-(1-bromovinyl)-2-(2′-fluoro-4′-glucuronide phenyl)-1,3-benzoxazol-5-glucuronide or a pharmaceutically acceptable salt thereof.

55. The compound according to claim 1, which is 7-(1-bromovinyl)-2-(2′-fluoro-4′-glucuronide phenyl)-1,3-benzoxazol-5-sulfate or a pharmaceutically acceptable salt thereof.

56. The compound according to claim 1, which is 7-(1-bromovinyl)-2-(2′-fluoro-4′-sulfate phenyl)-1,3-benzoxazol-5-glucuronide or a pharmaceutically acceptable salt thereof.

57. The compound according to claim 1, which is 7-(1-bromovinyl)-2-(2′-fluoro-4′-sulfate phenyl)-1,3-benzoxazol-5-sulfate or a pharmaceutically acceptable salt thereof.

58. The compound according to claim 1, which is 7-(1-bromovinyl)-2-(2′,3′-difluoro-4′-glucuronide phenyl)-1,3-benzoxazol-5-ol or a pharmaceutically acceptable salt thereof.

59. The compound according to claim 1, which is 7-(1-bromovinyl)-2-(2′,3′-difluoro-4′-sulfate phenyl)-1,3-benzoxazol-5-ol or a pharmaceutically acceptable salt thereof.

60. The compound according to claim 1, which is 7-(1-bromovinyl)-2-(2′,3′-difluoro-4′-hydroxyphenyl)-1,3-benzoxazol-5-glucuronide or a pharmaceutically acceptable salt thereof.

61. The compound according to claim 1, which is 7-(1-bromovinyl)-2-(2′,3′-difluoro-4′-hydroxyphenyl)-1,3-benzoxazol-5-sulfate or a pharmaceutically acceptable salt thereof.

62. The compound according to claim 1, which is 7-(1-bromovinyl)-2-(2′,3′-difluoro-4′-g lucuronide phenyl)-1,3-benzoxazol-5-glucuronide or a pharmaceutically acceptable salt thereof.

63. The compound according to claim 1, which is 7-(1-bromovinyl)-2-(2′,3′-difluoro-4′-glucuronide phenyl)-1,3-benzoxazol-5-sulfate or a pharmaceutically acceptable salt thereof.

64. The compound according to claim 1, which is 7-(1-bromovinyl)-2-(2′,3′-difluoro-4′-sulfate phenyl)-1,3-benzoxazol-5-glucuronide or a pharmaceutically acceptable salt thereof.

65. The compound according to claim 1, which is 7-(1-bromovinyl)-2-(2′,3′-difluoro-4′-sulfate phenyl)-1,3-benzoxazol-5-sulfate or a pharmaceutically acceptable salt thereof.

66. The compound according to claim 1, which is 7-allyl-2-(3′-fluoro-4′-glucuronide phenyl)-1,3-benzoxazol-5-ol or a pharmaceutically acceptable salt thereof.

67. The compound according to claim 1, which is 7-allyl-2-(3′-fluoro-4′-sulfate phenyl)-1,3-benzoxazol-5-ol or a pharmaceutically acceptable salt thereof.

68. The compound according to claim 1, which is 7-allyl-2-(3′-fluoro-4′-hydroxyphenyl)-1,3-benzoxazol-5-glucuronide or a pharmaceutically acceptable salt thereof.

69. The compound according to claim 1, which is 7-allyl-2-(3′-fluoro-4′-hydroxyphenyl)-1,3-benzoxazol-5-sulfate or a pharmaceutically acceptable salt thereof.

70. The compound according to claim 1, which is 7-allyl-2-(3′-fluoro-4′-glucuronide phenyl)-1,3-benzoxazol-5-glucuronide or a pharmaceutically acceptable salt thereof.

71. The compound according to claim 1, which is 7-allyl-2-(3′-fluoro-4′-glucuronide phenyl)-1,3-benzoxazol-5-sulfate or a pharmaceutically acceptable salt thereof.

72. The compound according to claim 1, which is 7-allyl-2-(3′-fluoro-4′-sulfate phenyl)-1,3-benzoxazol-5-glucuronide or a pharmaceutically acceptable salt thereof.

73. The compound according to claim 1, which is 7-allyl-2-(3′-fluoro-4′-sulfate phenyl)-1,3-benzoxazol-5-sulfate or a pharmaceutically acceptable salt thereof.

74. The compound according to claim 1, which is 2-(3′,5′-difluoro-4′-glucuronide phenyl)-7-vinyl-1,3-benzoxazol-5-ol or a pharmaceutically acceptable salt thereof.

75. The compound according to claim 1, which is 2-(3′,5′-difluoro-4′-sulfate phenyl)-7-vinyl-1,3-benzoxazol-5-ol or a pharmaceutically acceptable salt thereof.

76. The compound according to claim 1, which is 2-(3′,5′-difluoro-4′-hydroxy phenyl)-7-vinyl-1,3-benzoxazol-5-glucuronide or a pharmaceutically acceptable salt thereof.

77. The compound according to claim 1, which is 2-(3′,5′-difluoro-4′-hydroxyphenyl)-7-vinyl-1,3-benzoxazol-5-sulfate or a pharmaceutically acceptable salt thereof.

78. The compound according to claim 1, which is 2-(3′,5′-difluoro-4′-glucuronide phenyl)-7-vinyl-1,3-benzoxazol-5-gluguronide or a pharmaceutically acceptable salt thereof.

79. The compound according to claim 1, which is 2-(3′,5′-difluoro-4′-glucuronide phenyl)-7-vinyl-1,3-benzoxazol-5-sulfate or a pharmaceutically acceptable salt thereof.

80. The compound according to claim 1, which is 2-(3′,5′-difluoro-4′-sulfate phenyl)-7-vinyl-1,3-benzoxazol-5-glucuronide or a pharmaceutically acceptable salt thereof.

81. The compound according to claim 1, which is 2-(3′,5′-difluoro-4′-sulfate phenyl)-7-vinyl-1,3-benzoxazol-5-sulfate or a pharmaceutically acceptable salt thereof.

82. The compound according to claim 1, which is 2-(3′-fluoro-4′-glucuronide phenyl)-7-(1-fluorovinyl)-1,3-benzoxazol-5-ol or a pharmaceutically acceptable salt thereof.

83. The compound according to claim 1, which is 2-(3′-fluoro-4′-sulfate phenyl)-7-(1-fluorovinyl)-1,3-benzoxazol-5-ol or a pharmaceutically acceptable salt thereof.

84. The compound according to claim 1, which is 2-(3′-fluoro-4′-hydroxyphenyl)-7-(1-fluorovinyl)-1,3-benzoxazol-5-glucuronide or a pharmaceutically acceptable salt thereof.

85. The compound according to claim 1, which is 2-(3′-fluoro-4′-hydroxyphenyl)-7-(1-fluorovinyl)-1,3-benzoxazol-5-sulfate or a pharmaceutically acceptable salt thereof.

86. The compound according to claim 1, which is 2-(3′-fluoro-4′-glucuronide phenyl)-7-(1-fluorovinyl)-1,3-benzoxazol-5-glucuronide or a pharmaceutically acceptable salt thereof.

87. The compound according to claim 1, which is 2-(3′-fluoro-4′-glucuronide phenyl)-7-(1-fluorovinyl)-1,3-benzoxazol-5-sulfate or a pharmaceutically acceptable salt thereof.

88. The compound according to claim 1, which is 2-(3′-fluoro-4′-sulfate phenyl)-7-(1-fluorovinyl)-1,3-benzoxazol-5-glucuronide or a pharmaceutically acceptable salt thereof.

89. The compound according to claim 1, which is 2-(3′-fluoro-4′-sulfate phenyl)-7-(1-fluorovinyl)-1,3-benzoxazol-5-sulfate or a pharmaceutically acceptable salt thereof.

90. A compound which is a gluocuronide derivative, a sulfate derivative, or a glucuronide-sulfate derivative of:
a) 2-(5-hydroxy-1,3-benzoxazol-2-yl)benzene-1,4-diol;
b) 3-(5-hydroxy-1,3-benzoxazol-2-yl)benzene-1,2-diol;
c) 2-(3-fluoro-4-hydroxyphenyl)-1,3-benzoxazol-5-ol;
d) 2-(3-chloro-4-hydroxyphenyl)-1,3-benzoxazol-5-ol;
e) 2-(2-chloro-4-hydroxyphenyl)-1,3-benzoxazol-5-ol;
f) 2-(3-fluoro-4-hydroxyphenyl)-1,3-benzoxazol-6-ol;
g) 2-(3-tert-butyl-4-hydroxyphenyl)-1,3-benzoxazol-6-ol;
h) 2-(6-hydroxy-1,3-benzoxazol-2-yl)benzene-1,4-diol;
i) 3-(6-hydroxy-1,3-benzoxazol-2-yl)benzene-1,2-diol;
j) 4-(6-hydroxy-1,3-benzoxazol-2-yl)benzene-1,2-diol;
k) 2-(3-chloro-4-hydroxyphenyl)-1,3-benzoxazol-6-ol;
l) 4-(5-hydroxy-1,3-benzoxazol-2-yl)benzene-1,3-diol;
m) 4-(6-hydroxy-1,3-benzoxazol-2-yl)benzene-1,3-diol;
n) 6-chloro-2-(3-fluoro-4-hydroxyphenyl)-1,3-benzoxazol-5-ol;
o) 6-bromo-2-(3-fluoro-4-hydroxyphenyl)-1,3-benzoxazol-5-ol;
p) 6-chloro-2-(4-hydroxyphenyl)-1,3-benzoxazol-5-ol;
q) 5-chloro-2-(4-hydroxyphenyl)-1,3-benzoxazol-6-ol;
r) 7-bromo-2-(3-fluoro-4-hydroxyphenyl)-1,3-benzoxazol-5-ol;
s) 7-bromo-2-(2-fluoro-4-hydroxyphenyl)-1,3-benzoxazol-5-ol;
t) 7-bromo-2-(2,3-difluoro-4-hydroxyphenyl)-1,3-benzoxazol-5-ol;
u) 2-(4-hydroxyphenyl)-7-vinyl-1,3-benzoxazol-5-ol;
v) 7-(1,2-dibromoethyl)-2-(4-hydroxyphenyl)-1,3-benzoxazol-5-ol;
w) 7-(1-bromovinyl)-2-(4-hydroxyphenyl)-1,3-benzoxazol-5-ol;
x) 7-ethynyl-2-(4-hydroxyphenyl)-1,3-benzoxazol-5-ol;
y) 2-(4-hydroxyphenyl)-7-propyl-1,3-benzoxazol-5-ol;
z) 7-butyl-2-(4-hydroxyphenyl)-1,3-benzoxazol-5-ol;
aa) 7-cyclopentyl-2-(4-hydroxyphenyl)-1,3-benzoxazol-5-ol;
bb) ethyl 5-hydroxy-2-(4-hydroxyphenyl)-1,3-benzoxazole-7-carboxylate;
cc) 2-(4-hydroxyphenyl)-7-phenyl-1,3-benzoxazol-5-ol;
dd) 2-(4-hydroxyphenyl)-7-methoxy-1,3-benzoxazol-5-ol;
ee) 7-ethyl-2-(4-hydroxyphenyl)-1,3-benzoxazol-5-ol;
ff) 7-ethyl-2-(2-ethyl-4-hydroxyphenyl)-1,3-benzoxazol-5-ol;
gg) 5-hydroxy-2-(4-hydroxyphenyl)-1,3-benzoxazole-7-carbaldehyde;
hh) 7-(hydroxymethyl)-2-(4-hydroxyphenyl)-1,3-benzoxazol-5-ol;
ii) 7-(bromomethyl)-2-(4-hydroxyphenyl)-1,3-benzoxazol-5-ol;
jj) [5-hydroxy-2-(4-hydroxyphenyl)-1,3-benzoxazol-7-yl]acetonitrile;
kk) 7-(1-hydroxy-1-methylethyl)-2-(4-hydroxyphenyl)-1,3-benzoxazol-5-ol];
ll) 2-(4-hydroxyphenyl)-7-isopropenyl-1,3-benzoxazol-5-ol
mm) 2-(4-hydroxyphenyl)-7-isopropyl-1,3-benzoxazol-5-ol];
nn) 7-bromo-2-(4-hydroxy-3-(trifluoromethyl) phenyl)-1,3-benzoxazol-5-ol;
oo) 7-(2-furyl)-2-(4-hydroxyphenyl)-1,3-benzoxazol-5-ol;
pp) 2-(3-fluoro-4-hydroxyphenyl)-7-(2-furyl)-1,3-benzoxazol-5-ol;
qq) 2-(4-hydroxyphenyl)-7-thien-2-yl-1,3-benzoxazol-5-ol;
rr) 2-(4-hydroxyphenyl)-7-(1,3-thiazol-2-yl)-1,3-benzoxazol-5-ol;
ss) 2-(3-fluoro-4-hydroxyphenyl)-5-hydroxy-1,3-benzoxazole-7-carbonitrile;
tt) 4-bromo-2-(4-hydroxyphenyl)-7-methoxy-1,3-benzoxazol-5-ol;
uu) 4,6-dibromo-2-(4-hydroxyphenyl)-7-methoxy-1,3-benzoxazol-5-ol; or
vv) 7-bromo-2-(3,5-difluoro-4-hydroxyphenyl)-1,3-benzoxazol-5-ol;
or a pharmaceutically acceptable salt thereof.

91. A method of treating or inhibiting prostatitis or interstitial cystitis in a mammal in need thereof, which comprises providing to said mammal an effective amount of a compound of claim 1.

92. A method of treating or inhibiting inflammatory bowel disease, Crohn's disease, ulcerative proctitis, or colitis in a mammal in need thereof, which comprises providing to said mammal an effective amount of a compound of claim 1.

93. A method of treating or inhibiting prostatic hypertrophy, uterine leiomyomas, breast cancer, endometrial cancer, polycystic ovary syndrome, endometrial polyps, benign breast disease, adenomyosis, ovarian cancer, melanoma, prostrate cancer, colon cancer, glioma or astioblastomia in a mammal in need thereof, which comprises providing to said mammal an effective amount of a compound of claim 1.

94. A method of lowering cholesterol, triglycerides, Lp(a), or LDL levels; inhibiting or treating hypercholesteremia, hyperlipidemia, cardiovascular disease, atherosclerosis, hypertension, peripheral vascular disease, restenosis, or vasospasm; or inhibiting vascular wall damage from cellular events leading toward immune mediated vascular damage in a mammal in need thereof, which comprises providing to said mammal an effective amount of a compound of claim 1.

95. A method of providing cognition enhancement or neuroprotection; or treating or inhibiting senile dementias, Alzheimer's disease, cognitive decline, stroke, anxiety, or neurodegenerative disorders in a mammal in need thereof, which comprises providing to said mammal an effective amount of a compound of claim 1.

96. A method of treating or inhibiting free radical induced disease states in a mammal in need thereof, which comprises providing to said mammal an effective amount of a compound of claim 1.

97. A method of treating or inhibiting vaginal or vulvar atrophy, atrophic vaginitis, vaginal dryness, pruritus, dyspareunia, dysuria, frequent urination, urinary incontinence, urinary tract infections in a mammal in need thereof, which comprises providing to said mammal an effective amount of a compound of claim 1.

98. A method of treating or inhibiting vasomotor symptoms in a mammal in need thereof, which comprises providing to said mammal an effective amount of a compound of claim 1.

99. A method of inhibiting conception in a mammal in need thereof, which comprises providing to said mammal an effective amount of a compound of claim 1.

100. A method of treating or inhibiting arthritis in a mammal in need thereof, which comprises providing to said mammal an effective amount of a compound of claim 1.

101. The method according to claim 97, wherein the arthritis is rheumatoid arthritis, osteoarthritis, or spondyloarthropathies.

102. A method of treating or inhibiting joint swelling or erosion; or treating or inhibiting joint damage secondary to arthroscopic or surgical procedures in a mammal in need thereof, which comprises providing to said mammal an effective amount of a compound of claim 1.

103. A method of treating or inhibiting psoriasis or dermatitis in a mammal in need thereof, which comprises providing to said mammal an effective amount of a compound of claim 1.

104. A method of treating or inhibiting ischemia, reperfusion injury, asthma, pleurisy, multiple sclerosis, systemic lupus erythematosis, uveitis, sepsis, hemmorhagic shock, or type II diabetes in a mammal in need thereof, which comprises providing to said mammal an effective amount of a compound of claim 1.

105. A method of treating or inhibiting endometriosis in a mammal in need thereof, which comprises providing to said mammal an effective amount of a compound of claim 1.

106. A pharmaceutical composition which comprises a compound of claim 1, and a pharmaceutical carrier.
